(19) United States
(12) Patent Application Publication
(10) Pub. No.: US 2026/0137723 A1
(43) Pub. Date: May 21, 2026

(54) DEPRIVATION OF HUMAN PLURIPOTENT STEM CELL-DERIVED TRPV1+, MRGPRX1+ AND SCN9A+ SENSORY NEURONS AND THEIR FUNCTIONAL CHARACTERIZATION

(71) Applicant: The Johns Hopkins University, Baltimore, MD (US)

(72) Inventors: Gabsang LEE, Baltimore, MD (US); Xinzhong DONG, Baltimore, MD (US); Zhuolun Poppy WANG, Baltimore, MD (US)

(73) Assignee: The Johns Hopkins University, Baltimore, MD (US)

(21) Appl. No.: 19/385,524

(22) Filed: Nov. 11, 2025

Related U.S. Application Data
(63) Continuation-in-part of application No. PCT/US2024/056429, filed on Nov. 18, 2024.
(60) Provisional application No. 63/599,832, filed on Nov. 16, 2023.

(51) Int. Cl. A61K 35/30; A61P 19/02; C12N 5/0793; C12N 9/22; C12N 15/11

(52) U.S. Cl. CPC A61K 35/30; A61P 19/02; C12N 5/062; C12N 9/226; C12N 15/11; C12N 2310/20; C12N 2506/45

(57) ABSTRACT

Provided herein are methods of producing a population of pluripotent stem cell-derived sensory neurons (PSC-SNs) expressing a target gene associated with pain. In addition, provided herein are compositions and methods comprising populations of pluripotent stems cells for treating pain, osteoarthritis, and osteoarthritis related conditions.

### CROSS-REFERENCE TO RELATED APPLICATIONS

This application is a continuation-in-part of International Application No. PCT/US2024/056429 filed on Nov. 18, 2024, which claims priority to U.S. Application No. 63/599,832 filed on Nov. 16, 2023, this disclosure of which are hereby incorporated by reference in their entireties for all purposes.

### REFERENCE TO AN ELECTRONIC SEQUENCE LISTING

The contents of the electronic sequence listing (SENE_001_00US_SeqList_ST26.xml; Size: 676,224 bytes; and Date of Creation: Oct. 17, 2025) are herein incorporated by reference in its entirety.

### FIELD OF DISCLOSURE

The present disclosure relates generally to the fields of pain relief, joint pain, and osteoarthritis. More specifically, the present disclosure relates to populations of pluripotent stem cell-derived sensory neurons (PSC-SNs) expressing a target gene associated with pain or enriched for a biomarker. The disclosure further relates to methods of treating nociceptive pain, chronic pain, pruriception, and a nociceptive- or pruriceptive-mediated condition in a sensory neuron (SN).

### BACKGROUND

Chronic pain arises from the complex interplay of inflammatory signals that activate and sensitize nociceptors within injured tissues. The clinical failure of most analgesics stems from their mono-targeted mechanisms, which cannot modulate the diverse known and unknown pathways driving chronic pain. There is a critical need to evaluate pluripotent stem cell-derived sensory neurons (PSC-SNs) beyond conventional regenerative therapies. Rather than replacing damaged tissue, PSC-SNs can function as therapeutic agents targeting both the symptoms and pathogenesis of pain-related conditions. Moreover, this potential therapy provides a multi-targeted approach for chronic pain and a disease-modifying treatment for osteoarthritis, in which PSC-NNs can alleviate pain and support joint repair. Overall, these novel therapies offer strong potential for clinical translation in the treatment of chronic pain and bone-related disorders.

### BRIEF SUMMARY

In one aspect, provided herein is an isolated population of cells comprising pluripotent stem cell-derived sensory neurons (PSC-SNs) expressing at least one sensory neuron or pan neuronal marker. In some embodiments, the PSC-SNs respond to a nociceptive and pruriceptive stimulus. In some embodiments, the cells express TRPV1, SCN9A, MRGPRX1, or KCNQ2. In some embodiments, the cells express CD200^{high}. In some embodiments, at least 40%, at least 45%, at least 50%, at least 55%, at least 60%, at least 65%, at least 70%, at least 75%, at least 80%, at least 85%, at least 90%, at least 95%, at least 99% or 100% of the cells are CD200^{high}. In some embodiments, the PSC-SNs are derived from an animal or a human (hPSC-SNs). In some embodiments, the PSC-SNs comprise pluripotent stem cell-derived nociceptive sensory neurons (PSC-NSNs).

In another aspect, provided herein is a composition comprising the cells described herein, and at least one excipient.

In another aspect, provided herein is a method of treating pain or osteoarthritis, and/or promoting healing of injured tissues in a subject thereof, wherein the method comprises administering the cells described herein to the subject. In some embodiments, the method comprises administering to a joint of the subject. In some embodiments, the joint is a knee joint. In some embodiments, the pain comprises chronic pain, nociceptive pain, or pruriceptive pain. In some embodiments, the cells are administered to the subject at multiple intervals. In some embodiments, the subject is an animal or human. In some embodiments, the treated subject exhibits a reduction in pain by at least about 10%, at least about 20%, at least about 30%, at least about 40%, at least about 50%, at least about 60%, at least about 70%, at least about 80%, at least about 90%, or 100% in comparison to the subject prior to treatment or an untreated subject. In some embodiments, the treated subject exhibits a reduction in mechanical hypersensitivity by at least about 10%, at least about 20%, at least about 30%, at least about 40%, at least about 50%, at least about 60%, at least about 70%, at least about 80%, at least about 90%, or 100% in comparison to the subject prior to treatment or an untreated subject. In some embodiments, the treated subject exhibits a reduction in expression of matrix metalloproteinase 13 (MMP13) by at least about 10%, at least about 20%, at least about 30%, at least about 40%, at least about 50%, at least about 60%, at least about 70%, at least about 80%, at least about 90%, or 100% in comparison to the subject prior to treatment or an untreated subject.

In another aspect, provided herein is a composition for base editing target gene mutations, wherein the composition comprises a base editor and a guide RNA (gRNA) targeting at least one gene, wherein the at least one target gene comprises SCN9A, TRPV1, MRGPRX1, or KCNQ2. In some embodiments, the gRNA comprises a sequence selected from any one of SEQ ID NOs: 82-97. In some embodiments, the gRNA comprises a spacer sequence selected from any one of SEQ ID NOs: 17, 22, 27, 32, 35, 38, 41, 44, 47, 50, 53, 56, or 59-81. In some embodiments, the base editor comprises ABE8e, ABE8e(V106W), ABE9, ABE20, ABE7.10, SaKKHABE8e, SauriCas9-ABE8e, CjCas9-ABE8e, Nme2Cas9-ABE8e, or SaCas9-ABE8e. In some embodiments, the base editor comprises Cas9 endonuclease. In some embodiments, the Cas9 endonuclease comprises Type II Cas9.

In another aspect, provided herein is a method of editing at least one target gene comprising the composition described herein.

In another aspect, provided herein is a method of producing an isolated population of cells, wherein the population of cells comprises cells described herein, the method comprising contacting the cells with a composition for base editing target gene mutations, wherein the composition comprises a system comprising a base editor and a gRNA targeting at least one gene. In some embodiments, the cells have altered expression of the at least one target gene. In some embodiments, the cells have altered expression of the at least one target gene. In some embodiments, the least one target gene comprises SCN9A, TRPV1, MRGPRX1, or KCNQ2. In some embodiments, the gRNA comprises a sequence selected from any one of SEQ ID NOs: 82-97. In some embodiments, the gRNA comprises a spacer sequence selected from any one of SEQ ID NOs: 17, 22, 27, 32, 35, 38, 41, 44, 47, 50, 53, 56, or 59-81. In some embodiments, the base editor comprises ABE8e, ABE8e(V106W), ABE9, ABE20, ABE7.10, SaKKHABE8e, SauriCas9-ABE8e, CjCas9-ABE8e, Nme2Cas9-ABE8e, or SaCas9-ABE8e. In some embodiments, the base editor comprises Cas9 endonuclease. In some embodiments, the Cas9 endonuclease comprises Type II Cas9.

In another aspect, provided herein is a composition for base editing KCNQ2 mutations, wherein the composition comprises a system comprising a base editor and a gRNA targeting KCNQ2. In some embodiments, the gRNA comprises a sequence of SEQ ID NO: 83. In some embodiments, the gRNA comprises a spacer sequence selected from any one of SEQ ID NOs: 32, 35, 38, 41, 44, 47, 50, 53, 56, or 59-81. In some embodiments, the base editor comprises ABE8e, ABE8e(V106W), ABE9, ABE20, ABE7.10, SaKKHABE8e, SauriCas9-ABE8e, CjCas9-ABE8e, Nme2Cas9-ABE8e, or SaCas9-ABE8e.

In another aspect, provided herein is a method of editing KCNQ2 mutations comprising the composition described herein. In some embodiments, KCNQ2 function is restored. In some embodiments, the KCNQ2 mutations comprises a p.T730A or c.2188A>G mutation.

In another aspect, provided herein is a composition for silencing expression of TRPV1 and SCN9A, wherein the composition comprises a system comprising a Cas9 endonuclease and a gRNA targeting TRPV1 and SCN9A. In some embodiments, the gRNA comprises a sequence of SEQ ID NO: 86, 89-92, or 97. In some embodiments, the gRNA comprises a spacer sequence selected from any one of SEQ ID NOs: 22 or 27. In some embodiments, the Cas9 endonuclease comprises Type II Cas9.

In another aspect, provided herein is a method of editing TRPV1 or SCN9A comprising the composition described herein. In some embodiments, TRPV1 or SCN9A expression is reduced or eliminated.

In another aspect, provided herein is a method of producing an isolated population of cells comprising human pluripotent stem cell-derived nociceptive sensory neurons (hPSC-NSNs) expressing CD200^{high} with altered expression of KCNQ2, TRPV1, or SCN9A. In some embodiments, the cells are contacted with a composition for base editing KCNQ2 mutations, wherein the composition comprises a system comprising a base editor and a gRNA targeting KCNQ2. In some embodiments, the gRNA comprises a sequence of SEQ ID NO: 83. In some embodiments, the gRNA comprises a spacer sequence selected from any one of SEQ ID NOs: 32, 35, 38, 41, 44, 47, 50, 53, 56, or 59-81. In some embodiments, the base editor comprises ABE8e, ABE8e(V106W), ABE9, ABE20, ABE7.10, SaKKHABE8e, SauriCas9-ABE8e, CjCas9-ABE8e, Nme2Cas9-ABE8e, or SaCas9-ABE8e. In some embodiments, the cells are contacted with a composition for silencing expression of TRPV1 and SCN9A, wherein the composition comprises a system comprising a Cas9 endonuclease and a gRNA targeting TRPV1 and SCN9A. In some embodiments, the gRNA comprises a sequence of SEQ ID NO: 86, 89-92, or 97. In some embodiments, the gRNA comprises a spacer sequence selected from any one of SEQ ID NOs: 22 and 27. In some embodiments, the Cas9 endonuclease comprises Type II Cas9.

### BRIEF DESCRIPTION OF THE DRAWINGS

The accompanying figures, which are incorporated herein and form a part of the specification, illustrate some, but not the only or exclusive, example embodiments and/or features. It is intended that the embodiments and figures disclosed herein are to be considered illustrative rather than limiting.

FIGS. 1A-1D illustrate generating PSC reporter lines.

FIGS. 2A-2E illustrate characterizing the transcriptional landscape of MRGPRX1::GFP, SCN9A::GFP, and TRPV1::GFP sensory neurons.

FIGS. 3A-3F illustrate purifiable and excitable populations of hPSC-derived MRGPRX1+ and TRPV1+ sensory neurons.

FIGS. 4A-4L illustrate FACS-purified TRPV1+, SCN9A+, and MRGPRX1+ hPSC-derived sensory neurons responding to various pain- and itch-inducing ligands.

FIGS. 5A-5S illustrate in vitro optimization and functional validation of candidate CRISPR sgRNAs to enable TRPV1 knockout, and SCN9A knockout.

FIGS. 6A-6I illustrate single cell RNA-seq data and CD200 as a nociceptor marker.

FIGS. 7A-7DD illustrate a KCNQ2 as a gene therapy target gene conferring robust pain-resilience effects in hPSC-derived sensory neurons and the KCNQ2 gene therapy efficacy in a rodent model resulting in bone remodeling.

FIGS. 8A-8R illustrate CD200+ sensory neuron injection to intercept pain to treat localized pain and improve the bone remodeling by injecting hPSC-derived pain-sensing neurons.

FIGS. 9A-9K illustrate in vivo survival and function of SCN9A+, TRPV1+, and MRGPRX1+ neurons.

FIGS. 10A-10I show CD200^{high} hPSC-NNs promote healthy joint homeostasis by depleting inflammatory factors and secreting factors that facilitate tissue repair.

FIG. 10A shows Expression of ATF3 (red) in transplanted CD200^{low} hPSC-SNs and CD200^{high} hPSC-NNs (green) in knee sections at 5 and 10 weeks post-injection (scale bar, 10 μm). FIG. 10C shows a quantification of the proportion of CD200^{low} hPSC-SNs or CD200^{high} hPSC-NNs expressing ATF3.9

FIG. 10B shows representative Ca²⁺ transients of CD200^{low} hPSC-SNs and CD200^{high} hPSC-NNs acutely exposed to osteoarthritis (OA) hSF or pooled. FIG. 10 D shows Quantification of portion of responsive neurons.

FIG. 10E shows a schematic workflow of proteomic analysis of supernatants from CD200^{high} hPSC-NNs and CD200^{low} hPSC-SNs following overnight incubation with OA hSF or pooled healthy hSF healthy hSF (bold traces show the mean).

FIGS. 10F-10G show a reactome pathway analysis showing downregulated (FIG. 10F) and upregulated (FIG. 10G) pathways in CD200^{high} hPSC-NNs versus CD200^{low} hPSC-SNs incubated in OA hSF.

FIGS. 10H-10I show volcano plots of proteins depleted (FIG. 10H) or enriched (FIG. 10I) in OA hSF following incubation with CD200^{high} hPSC-NNs versus CD200^{low} hPSC-SNs.

FIGS. 11A-11I CD200^{high} hPSC-NNs restored the disrupted proteome in OA mice, promoting an anti-inflammatory and reparative microenvironment.

FIG. 11A shows a schematic workflow of proteomic analysis of knee joints harvested 10 weeks post-injection from ACLT animals injected with viable CD200^{high} hPSC-NNs (ACLT-CD200^{high}), ACLT animals injected with heat-killed hPSC-SNs (ACLT-heat killed), and sham-operated animals injected with heat-killed hPSC-SNs (sham-heat killed). FIG. 11B shows a venn diagram exhibiting differentially expressed proteins, highlighting similarity between the proteomes of sham-heat killed and ACLT-CD200^{high} animals. FIG. 11C shows a schematic representation of the dual mechanism of action of CD200^{high} hPSC-NNs in OA mice.

FIG. 11D shows volcano plots of proteins enriched in ACLT-heat killed versus sham-heat killed animals (left) and reversed by CD200^{low} hPSC-NNs 10 weeks post-injection (right) (shared proteins are boxed). FIG. 11E shows volcano plots of proteins downregulated in ACLT-heat killed versus sham-heat killed animals (left) and restored by CD200^{high} hPSC-NNs 10 weeks post-injection (right) (shared proteins are boxed).

FIGS. 11F and 11G show reactome pathways enriched in ACLT-heat killed versus sham-heat killed animals (FIG. 11F) and reversed by CD200^{high} hPSC-NNs 10 weeks post-injection (FIG. 11G). FIGS. 11H and 11I show reactome pathways downregulated in ACLT-heat killed versus sham-heat killed animals (FIG. 1111) and restored by CD200^{high} hPSC-NNs 10 weeks post-injection (FIG. 11I).

FIG. 12 shows the CRISPR-Cas9 ribonucleoprotein (RNP) and homology-directed recombination (HDR) template design. The PAM sequence is labeled in blue, restriction enzyme sites are shown in yellow, and the genomic sequence is shown in green. A silent mutagenesis is designed on the right homology arm for the TRPV1 locus. The PAM sequence for all loci is designed to be located within 10 bp of the insertion site.

FIGS. 13A-H show animal models and results for evaluation of a targeted base editor.

### DETAILED DESCRIPTION

All publications, patents and patent applications, including any drawings and appendices, are herein incorporated by reference to the same extent as if each individual publication or patent application was specifically and individually indicated to be incorporated by reference.

The following description includes information that may be useful in understanding the present disclosure. It is not an admission that any of the information provided herein is prior art or relevant to the presently claimed disclosures, or that any publication specifically or implicitly referenced is prior art.

### Definitions

The terms, “a,” “an,” and “the,” as used herein, include plural references unless the context clearly dictates otherwise.

The terms, “or” and “and/or,” as used herein, include any, and all, combinations of one or more of the associated listed items.

The terms, “including,” “includes,” “included,” and other forms, are not limiting.

The terms, “comprise” and its grammatical equivalents, as used herein, specify the presence of stated features, integers, steps, operations, elements, and/or components, but do not preclude the presence or addition of one or more other features, integers, steps, operations, elements, components, and/or groups thereof.

The term, “about,” as used herein in reference to a number or range of numbers, is understood to mean the stated number and numbers +/−10% thereof, or 10% below the lower listed limit and 10% above the higher listed limit for the values listed for a range.

The term “base editing enzyme,” as used herein, refers to a protein, polypeptide or fragment thereof that is capable of catalyzing the chemical modification of a nucleobase of a deoxyribonucleotide or a ribonucleotide. Such a base editing enzyme, for example, is capable of catalyzing a reaction that modifies a nucleobase that is present in a nucleic acid molecule, such as DNA or RNA (single stranded or double stranded). Non-limiting examples of the type of modification that a base editing enzyme is capable of catalyzing includes converting an existing nucleobase to a different nucleobase, such as converting a cytosine to a guanine or thymine or converting an adenine to a guanine, hydrolytic deamination of an adenine or adenosine, or methylation of cytosine (e.g., CpG, CpA, CpT or CpC). A base editing enzyme itself may or may not bind to the nucleic acid molecule containing the nucleobase.

The term “base editor,” as used herein, refers to a fusion protein comprising a base editing enzyme linked to an effector protein. The base editing enzyme may be referred to as a fusion partner. The base editing enzyme can differ from a naturally occurring base editing enzyme. It is understood that any reference to a base editing enzyme herein also refers to a base editing enzyme variant. The base editor is functional when the effector protein is coupled to a guide nucleic acid. The guide nucleic acid imparts sequence specific activity to the base editor. By way of non-limiting example, the effector protein may comprise a catalytically inactive effector protein. Also, by way of non-limiting example, the base editing enzyme may comprise deaminase activity. Additional base editors are described herein.

The terms “complementary” and “complementarity,” as used herein, with reference to a nucleic acid molecule or nucleotide sequence, refer to the characteristic of a polynucleotide having nucleotides that base pair with their Watson-Crick counterparts (C with G; or A with T or U) in a reference nucleic acid. For example, when every nucleotide in a polynucleotide forms a base pair with a reference nucleic acid, that polynucleotide is said to be 100% complementary to the reference nucleic acid. In a double stranded DNA or RNA sequence, the upper (sense) strand sequence is in general, understood as going in the direction from its 5′- to 3′-end, and the complementary sequence is thus understood as the sequence of the lower (antisense) strand in the same direction as the upper strand. Following the same logic, the reverse sequence is understood as the sequence of the upper strand in the direction from its 3′- to its 5′-end, while the ‘reverse complement’ sequence or the ‘reverse complementary’ sequence is understood as the sequence of the lower strand in the direction of its 5′- to its 3′-end. Each nucleotide in a double stranded DNA or RNA molecule that is paired with its Watson-Crick counterpart called its complementary nucleotide.

The term “cis cleavage,” as used herein, refers to cleavage (hydrolysis of a phosphodiester bond) of a target nucleic acid by an effector protein complexed with a guide nucleic acid (e.g., an RNP complex), wherein at least a portion of the guide nucleic acid is hybridized to at least a portion of the target nucleic acid. Cleavage may occur within or directly adjacent to the region of the target nucleic acid that is hybridized to the guide nucleic acid.

The term “cleavage assay,” as used herein, refers to an assay designed to visualize, quantitate, or identify cleavage of a nucleic acid. In some cases, the cleavage activity may be cis-cleavage activity. In some cases, the cleavage activity may be trans-cleavage activity.

The terms “cleave,” “cleaving,” and “cleavage,” as used herein, with reference to a nucleic acid molecule or nuclease activity of an effector protein, refer to the hydrolysis of a phosphodiester bond of a nucleic acid molecule that results in breakage of that bond. The result of this breakage can be a nick (hydrolysis of a single phosphodiester bond on one side of a double-stranded molecule), single strand break (hydrolysis of a single phosphodiester bond on a single-stranded molecule) or double strand break (hydrolysis of two phosphodiester bonds on both sides of a double-stranded molecule) depending upon whether the nucleic acid molecule is single-stranded (e.g., ssDNA or ssRNA) or double-stranded (e.g., dsDNA) and the type of nuclease activity being catalyzed by the effector protein.

The term “clustered regularly interspaced short palindromic repeats (CRISPR),” as used herein, refers to a segment of DNA found in the genomes of certain prokaryotic organisms, including some bacteria and archaea, that includes repeated short sequences of nucleotides interspersed at regular intervals between unique sequences of nucleotides derived from the DNA of a pathogen (e.g., virus) that had previously infected the organism and that functions to protect the organism against future infections by the same pathogen.

The terms “CRISPR RNA” or “crRNA,” as used herein, refer to a type of guide nucleic acid, wherein the nucleic acid is RNA comprising a first sequence that is capable of interacting with an effector protein either directly (by being bound by an effector protein) or indirectly (e.g., by hybridization with a second nucleic acid molecule that can be bound by an effector, such as a tracrRNA); and a second sequence that hybridizes to a target sequence of a target nucleic acid. In some embodiments, the first sequence is referred to as a repeat sequence and the second sequence is referred to as a spacer sequence. The first sequence and the second sequence are directly connected to each other or by a linker.

The terms “expression unit” and “expression cassette” are used interchangeably herein and denote a nucleic acid segment encoding a polypeptide of interest and capable of providing expression of the nucleic acid segment in a host cell. An expression unit typically comprises a transcription promoter, an open reading frame encoding the polypeptide of interest, and a transcription terminator, all in operable configuration. In addition to a transcriptional promoter and terminator, an expression unit can further include other nucleic acid segments such as, e.g., an enhancer or a polyadenylation signal. In certain embodiments, for example, an “expression cassette” comprises a DNA coding sequence operably linked to a promoter. “Operably linked” refers to a juxtaposition wherein the components so described are in a relationship permitting them to function in their intended manner. For instance, a promoter is operably linked to a coding sequence (or the coding sequence can also be said to be operably linked to the promoter) if the promoter affects its transcription or expression. In certain embodiments, for example, an expression cassette may be a genetic sequence within a vector which can express an RNA, and subsequently a protein. The nucleic acid cassette contains the gene of interest, e.g., a gene-regulating system. The nucleic acid cassette is positionally and sequentially oriented within the vector such that the nucleic acid in the cassette can be transcribed into RNA, and when necessary, translated into a protein or a polypeptide, undergo appropriate post-translational modifications required for activity in the transformed cell, and be translocated to the appropriate compartment for biological activity by targeting to appropriate intracellular compartments or secretion into extracellular compartments. Preferably, the cassette has its 3′ and 5′ ends adapted for ready insertion into a vector, e.g., it has restriction endonuclease sites at each end. The cassette can be removed and inserted into a plasmid or viral vector as a single unit

The terms “fusion protein,” or “fusion effector protein,” as used herein, refer to a protein comprising at least two heterologous polypeptides. The fusion protein may comprise one or more effector proteins and fusion partners. In some embodiments, an effector protein and fusion partner are not found connected to one another as a native protein or complex that occurs together in nature.

The term “functional domain,” as used herein, refers to a region of one or more amino acids in a protein that is required for an activity of the protein, or the full extent of that activity, as measured in an in vitro assay. Activities include, but are not limited to nucleic acid binding, nucleic acid modification, nucleic acid cleavage, protein binding. The absence of the functional domain, including mutations of the functional domain, would abolish or reduce activity.

The term “guide nucleic acid,” as used herein, refers to a nucleic acid comprising: a first nucleotide sequence that is capable of being non-covalently bound by an effector protein; and a second nucleotide sequence that hybridizes to a target nucleic acid. When in a complex with one or more polypeptides described herein (e.g., an RNP complex), a guide nucleic acid can impart sequence selectivity to the complex when the complex interacts with a target nucleic acid. The first sequence may be referred to herein as a repeat sequence. The second sequence may be referred to herein as a spacer sequence. The term, “guide nucleic acid,” may be used interchangeably herein with the term “guide RNA” (gRNA) however it is understood that guide nucleic acids may comprise deoxyribonucleotides (DNA), ribonucleotides (RNA), a combination thereof (e.g., RNA with a thymine base), biochemically or chemically modified nucleobases (e.g., one or more engineered modifications described herein), or combinations thereof.

The term “heterologous,” as used herein, means a nucleotide or polypeptide sequence that is not found in a native nucleic acid or protein, respectively. In some embodiments, fusion proteins comprise an effector protein and a fusion partner protein, wherein the fusion partner protein is heterologous to an effector protein. These fusion proteins may be referred to as a “heterologous protein.” A protein that is heterologous to the effector protein is a protein that is not covalently linked via an amide bond to the effector protein in nature. In some embodiments, a heterologous protein is not encoded by a species that encodes the effector protein. In some embodiments, the heterologous protein exhibits an activity (e.g., enzymatic activity) when it is linked to the effector protein. In some embodiments, the heterologous protein exhibits increased or reduced activity (e.g., enzymatic activity) when it is linked to the effector protein, relative to when it is not linked to the effector protein. In some embodiments, the heterologous protein exhibits an activity (e.g., enzymatic activity) that it does not exhibit when it is linked to the effector protein. A guide nucleic acid may comprise a first sequence and a second sequence, wherein the first sequence and the second sequence are not found covalently linked via a phosphodiester bond in nature. Thus, the first sequence is considered to be heterologous with the second sequence, and the guide nucleic acid may be referred to as a heterologous guide nucleic acid.

The term “linked” when used in reference to biopolymers (e.g., nucleic acids, polypeptides) refers to being covalently connected. In some embodiments, two polymers are linked by at least a covalent bond. In some embodiments, two nucleic acids are linked by at least one nucleotide. In some embodiments, two nucleic acids are linked by at least one amino acid. The terms “fused” and “linked” are used interchangeably herein.

The term “linker,” as used herein, refers to a covalent bond or molecule that links a first polypeptide to a second polypeptide (e.g., by an amide bond, or one or more amino acids) or a first nucleic acid to a second nucleic acid (e.g., by a phosphodiester bond, or one or more nucleotides).

The term “modified target nucleic acid,” as used herein, refers to a target nucleic acid, wherein the target nucleic acid has undergone a modification, for example, after contact with an effector protein. In some cases, the modification is an alteration in the sequence of the target nucleic acid. In some cases, the modified target nucleic acid comprises an insertion, deletion, or replacement of one or more nucleotides compared to the unmodified target nucleic acid.

The terms “non-naturally occurring” and “engineered,” as used herein, are used interchangeably and indicate the involvement of the hand of man. The terms, when referring to a nucleic acid, nucleotide, protein, polypeptide, peptide or amino acid, refer to a nucleic acid, nucleotide, protein, polypeptide, peptide or amino acid that is at least substantially free from at least one other feature with which it is naturally associated in nature and as found in nature, and/or contains a modification (e.g., chemical modification, nucleotide sequence, or amino acid sequence) that is not present in the naturally occurring nucleic acid, nucleotide, protein, polypeptide, peptide, or amino acid. The terms, when referring to a composition or system described herein, refer to a composition or system having at least one component that is not naturally associated with the other components of the composition or system. By way of a non-limiting example, a composition may include an effector protein and a guide nucleic acid that do not naturally occur together. Conversely, and as a non-limiting further clarifying example, an effector protein or guide nucleic acid that is “natural,” “naturally-occurring,” or “found in nature” includes an effector protein and a guide nucleic acid from a cell or organism that have not been genetically modified by the hand of man.

The term “nucleic acid expression vector,” as used herein, refers to a nucleic acid that can be used to express a nucleic acid of interest.

The term “nuclear localization signal (NLS),” as used herein, refers to an entity (e.g., peptide) that facilitates localization of a nucleic acid, protein, or small molecule to the nucleus, when present in a cell that contains a nuclear compartment.

The term “nuclease activity,” as used herein, refers to the catalytic activity that results in nucleic acid cleavage (e.g., ribonuclease activity (ribonucleic acid cleavage), or deoxyribonuclease activity (deoxyribonucleic acid cleavage), etc.).

The terms “partner protein,” “fusion partner,” or “fusion partner protein” as used herein, refer to a protein, polypeptide or peptide that is linked to an effector protein or capable of being proximal to an effector protein. In some embodiments, a fusion partner that is capable of being proximal to an effector protein is a fusion partner that is capable of binding a guide nucleic acid, wherein the effector protein is also capable of binding the guide nucleic acid. In some embodiments, a fusion partner directly interacts with (e.g., binds to/by) an effector protein. In some embodiments, a fusion partner indirectly interacts with an effector protein (e.g., through another protein or moiety).

The term “pharmaceutically acceptable excipient, carrier or diluent,” as used herein, refers to any substance formulated alongside the active ingredient of a pharmaceutical composition that allows the active ingredient to retain biological activity and is non-reactive with the subject's immune system. Such a substance can be included for the purpose of long-term stabilization, bulking up solid formulations that contain potent active ingredients in small amounts, or to confer a therapeutic enhancement on the active ingredient in the final dosage form, such as facilitating absorption, reducing viscosity, or enhancing solubility. The selection of appropriate substance can depend upon the route of administration and the dosage form, as well as the active ingredient and other factors. Compositions having such substances can be formulated by well-known conventional methods (see, e.g., Remington, The Science and Practice of Pharmacy 23^{rd} edition, A. Adejare, ed., Elsevier Publishing Co., 2020).

The terms, “promoter” and “promoter sequence,” as used herein, refer to a DNA regulatory region capable of binding RNA polymerase and initiating transcription of a downstream (3′ direction) coding or non-coding sequence. A transcription initiation site, as well as protein binding domains responsible for the binding of RNA polymerase, can also be found in a promoter region. Eukaryotic promoters will often, but not always, contain “TATA” boxes and “CAT” boxes. Various promoters, including inducible promoters, may be used to drive expression by the various vectors of the present disclosure.

The term “protospacer adjacent motif” and “PAM,” as used herein, refers to a nucleotide sequence found in a target nucleic acid that directs an effector protein to modify the target nucleic acid at a specific location. In some embodiments, a PAM sequence is required for a complex of an effector protein and a guide nucleic acid (e.g., an RNP complex) to hybridize to and edit the target nucleic acid. In some embodiments, the complex does not require a PAM to edit the target nucleic acid.

In some embodiments, the term “region” as used herein may be used to describe a portion of, or all of, a corresponding sequence, for example, a spacer region is understood to comprise a portion of or all of a spacer sequence.

The term, “regulatory element,” used herein, refers to transcriptional and translational control sequences, such as promoters, enhancers, polyadenylation signals, terminators, protein degradation signals, and the like, that provide for and/or regulate transcription of a non-coding sequence (e.g., a guide nucleic acid) or a coding sequence (e.g., effector proteins, fusion proteins, and the like) and/or regulate translation of an encoded polypeptide.

The term, “repeat sequence,” as used herein, refers to a sequence of nucleotides in a guide nucleic acid that is capable of, at least partially, interacting with an effector protein.

The terms, “ribonucleotide protein complex” and “RNP” as used herein, refer to a complex of one or more nucleic acids and one or more polypeptides described herein. While the term utilizes “ribonucleotides” it is understood that the one or more nucleic acid may comprise deoxyribonucleotides (DNA), ribonucleotides (RNA), a combination thereof (e.g., RNA with a thymine base), biochemically or chemically modified nucleobases (e.g., one or more engineered modifications described herein), or combinations thereof.

As used herein, the term “sequence identity” refers to a relationship between two or more polynucleotide sequences or between two or more polypeptide sequences. In certain embodiments, for example, “sequence identity” or, for example, comprising a “sequence 50% identical to” may refer to the extent that sequences are identical on a nucleotide-by-nucleotide basis or an amino acid-by-amino acid basis over a window of comparison. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. When a position in one sequence is occupied by the same nucleic acid base or amino acid residue in the corresponding position of the comparator sequence, the sequences are said to be “identical” at that position. The percentage sequence identity is calculated by determining the number of positions at which the identical nucleic acid base or amino acid residue occurs in both sequences to yield the number of identical positions. The number of identical positions is then divided by the total number of positions in the comparison window and multiplied by 100 to yield the percentage of sequence identity. Percentage of sequence identity is determined by comparing two optimally aligned sequences over a comparison window. The comparison window for polynucleotide sequences can be, for instance, at least 20, 30, 40, 50, 60, 70, 80, 90, 100, 110, 120, 130, 140, 150, 160, 170, 180, 190, 200, 300, 400, 500, 600, 700, 800, 900 or 1000 or more nucleic acids in length. The comparison window for polypeptide sequences can be, for instance, at least 20, 30, 40, 50, 60, 70, 80, 90, 100, 110, 120, 130, 140, 150, 160, 170, 180, 190, 200, 300 or more amino acids in length. In order to optimally align sequences for comparison, the portion of a polynucleotide or polypeptide sequence in the comparison window can comprise additions or deletions termed gaps while the reference sequence is kept constant. Alignment for purposes of determining percent amino acid sequence identity can be achieved in various ways that are within the skill in the art, for instance, using publicly available computer software such as EMBOSS MATCHER, EMBOSS WATER, EMBOSS STRETCHER, EMBOSS NEEDEE, EMBOSS LALIGN, BLAST, BLAST-2, ALIGN, ALIGN-2, CLUSTAL OMEGA, or Megalign (DNASTAR) software. Those skilled in the art can determine appropriate parameters for measuring alignment, including any algorithms needed to achieve maximal alignment over the full length of the sequences being compared.

The terms, “% identical,” “% identity,” and “percent identity,” or grammatical equivalents thereof, refer to the extent to which two sequences (nucleotide or amino acid) have the same residue at the same positions in an alignment. For example, “an amino acid sequence is X % identical to SEQ ID NO: Y” can refer to % identity of the amino acid sequence to SEQ ID NO: Y and is elaborated as X % of residues in the amino acid sequence are identical to the residues of sequence disclosed in SEQ ID NO: Y. Generally, computer programs can be employed for such calculations. Illustrative programs that compare and align pairs of sequences, include ALIGN (Myers and Miller, Comput Appl Biosci. 1988 March; 4(1):11-7), FASTA (Pearson and Lipman, Proc Natl Acad Sci USA. 1988 April; 85(8):2444-8; Pearson, Methods Enzymol. 1990; 183:63-98) and gapped BLAST (Altschul et al., Nucleic Acids Res. 1997 Sep. 1; 25(17):3389-40), BLASTP, BLASTN, or GCG.

As used herein, the term “subject” refers to any subject, e.g., a human or a non-human mammal, for whom diagnosis, prognosis, or therapy is desired. The term “subject” may mean a human or non-human mammal affected, likely to be affected, or suspected to be affected with a disease. The terms “subject” and “patient” are used interchangeably herein. In some embodiments, a subject is a mammal. A mammal includes primates, such as humans, monkeys, chimpanzee, and apes, and non-primates such as domestic animals, including laboratory animals (such as rabbits and rodents, e.g., guinea pig, rat, or mouse) and household pets and farm animals (e.g., cats, dogs, swine, cattle, sheep, goats, horses, rabbits), and non-domestic animals, such as wildlife, birds, reptile, fish, or the like.

As used herein, the terms “nucleic acid”, “nucleic acid molecule”, or “polynucleotide” refer to deoxyribonucleotides or ribonucleotides and polymers thereof in either single- or double-stranded form. Unless specifically limited, the terms encompass nucleic acids containing analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions can be achieved by generating sequences in which the third position of one or more selected (or all) codons is substituted with mixed-base and/or deoxyinosine residues (Batzer et al. (1991) Nucleic Acid Res. 19:5081; Ohtsuka et al. (1985) J. Biol. Chem. 260:2605-2608; Cassol et al. (1992); Rossolini et al. (1994) Mol. Cell. Probes 8:91-98). The term nucleic acid is used interchangeably with gene, cDNA, and mRNA encoded by a gene. As used herein, the terms “nucleic acid”, “nucleic acid molecule”, or “polynucleotide” are intended to include DNA molecules (e.g., cDNA or genomic DNA), RNA molecules (e.g., mRNA), analogs of the DNA or RNA generated using nucleotide analogs, and derivatives, fragments and homologs thereof.

The term “expression vector,” as used herein, refers to a nucleic acid molecule, linear or circular, comprising one or more expression units. In addition to one or more expression units, an expression vector can also include additional nucleic acid segments such as, for example, one or more origins of replication or one or more selectable markers. Expression vectors are generally derived from plasmid or viral DNA, or can contain elements of both.

As used herein, the term “transformation”, “transfection”, and “transduction” refer to the transfer of nucleic acid (i.e., a nucleotide polymer) into a cell.

The terms “amino-terminal” and “carboxyl-terminal” are used herein to denote positions within polypeptides. Where the context allows, these terms are used with reference to a particular sequence or portion of a polypeptide to denote proximity or relative position.

Throughout the present disclosure, the expression of an indicated protein by a cell or population of cells may include reference to various expression indicators such as “+” (e.g., CD200+), “−” (e.g., CD200−), “high” (e.g., CD200^{high}), and “low” (e.g., CD200^{low}). Herein, the various expression indicators refer to the presence or absence of the indicated protein (e.g., “+” or “−”, respectively) or the relative level of protein expression as measured by a convention protein expression assay (e.g., flow cytometry, fluorescence active cell sorting (FACS), or Western blot). Unless otherwise indicated, protein expression as described throughout the present application is determined by FACS.

A positive (+) indicator refers to a detectable level of expression of the indicated protein by FACS. A population of cells that is positive (+) for a particular protein may be further divided into populations of “low” and/or “high” subpopulations.

A subpopulation of “low” cells expresses the indicated protein but at a lower level than the other cells in the population (e.g., at least 60%, at least 70%, at least 80%, or at least 90% lower than the expression level of the other cells in the population) or at a lower level than expression in a control cell population.

A subpopulation of “high” cells expresses the indicated protein at a higher level than the other cells in the population (e.g., at least 40%, at least 50%, at least 60%, at least 70%, at least 80%, or at least 90% higher than the expression level of the other cells in the population) or at a higher level than expression in a control cell population.

A negative (−) indicator refers to an absence of expression of the indicated protein, or an expression level of the indicated protein that is below the limit of detection for the particular detection assay (e.g., below the limit of detection for a particular fluorescent antibody and/or flow cytometer).

### Overview

The human somatosensory system is comprised of diverse subtypes of sensory neurons, with each specialized in detecting specific modalities such as touch, pressure, temperature, vibration, pain, and body position. Among these, nociceptors detect potentially harmful stimuli, including noxious chemicals, thermal, and mechanical stimuli, through their heterogeneous expression of ion channels and receptors. In pathological states, however, persistent exposure to pro-inflammatory mediators, including cytokines such as TNF-α, PGE2, and IL-6 and neurotrophic factors like NGF, induces nociceptor hyperexcitability and upregulation of pro-nociceptive genes, which drive peripheral and central sensitization and result in chronic pain that is refractory to traditional mono-targeted analgesics.

Osteoarthritis (OA) exemplifies the convergence of inflammation and chronic pain. In early osteoarthritis, inflammatory cytokines drive osteoclast-mediated subchondral bone loss, while later stages are marked by abnormal osteoblast activity and development of subchondral sclerosis. This altered microenvironment creates a self-perpetuating axis that promotes aberrant nerve sprouting, amplifies pain, and sustains joint inflammation. Although the inflammatory nature of OA-associated pain is well recognized, current therapeutic strategies, including nonsteroidal anti-inflammatory drugs (NSAIDs), COX2 inhibitors, and anti-NGF antibodies, target singular pathways and fail to address the complex neuro-immune interactions that drive disease progression.

Efforts to model nociceptive signaling in chronic pain using heterologous expression systems or conventional human pluripotent stem cell-derived sensory neurons (hPSC-SNs) have faced some limitations. For example, heterologous systems lack the native neuronal machinery, while hPSC-SNs are highly heterogeneous, thus complicating the isolation and study of nociceptor subtypes. Provided herein are exemplary cell-based therapeutic strategies that leverage purified hPSC-derived nociceptive neurons (hPSC-NNs) as biological decoys to target the pathological neuro-immune axis in OA.

The intrinsic sensitivity of hPSC-NNs to inflammatory factors, which are conferred by their broad expression of most, if not all, receptors and ion channels of endogenous human nociceptors that cover known and unknown pain-relevant pathways, can be harnessed therapeutically. For instance, purified hPSC-NNs can be used to sequester inflammatory mediators and modulate the local joint microenvironment, thereby alleviating pain and reversing the underlying pathogenesis of OA.

Furthermore, it is estimated that over one-third of the world's population suffers from persistent or recurrent pain/itching caused by neurological disorders, diabetes, car accidents, war injuries, chemotherapy, etc. Most drugs on the market for pain sensation have undesired side effects because their targets exist both inside and outside the pain pathways, which makes the nociceptive and pruriceptive neurons as attractive targets for novel pharmacological strategies.

In addition to the urgent need for new painkillers without side effects, it is also important to develop a new humanized model system. To address this issue, some embodiments of the present disclosure provide a generation of human transient receptor potential cation channel subfamily V member 1 (TRPV1)+, Mas-related G protein-coupled receptor X1 (MRGPRX1)+ and sodium voltage-gated channel alpha subunit 9 (SCN9A)+ neurons from human pluripotent stem cells (hPSCs), followed by detailed functional characterization. When injected into host, these hPSCs can survive and rapidly generate extended axons.

Recent studies have revealed that nociceptive and pruriceptive neurons encompass a remarkably heterogeneous population that entails various transductions of noxious stimuli through numerous ion channels, membrane receptors, signaling molecules and neuropeptides and neurotransmitters, which hamper detailed understanding of human nociception as well as analgesic drug development. The transient receptor potential cation channel subfamily V member 1 (TRPV1) and sodium voltage-gated channel alpha subunit 9 (SCN9A, encoding Nav1.7) are promising targets of novel pain inhibitors, mainly because of their restricted expression in primary nociceptive neurons. However, the functional properties of human TRPV1 and SCN9A cannot be fully inferred from rodent analogs, owing to cross-species variations in their agonist activity and receptor function. While there have been innovations and advances in modelling and measuring pain in animals, it is also important to acquire TRPV1 and SCN9A expressing human nociceptive neurons.

Because persistent pain is often primed with peripheral pathological conditions, such as tissue inflammation and nerve injury, and its maintenance is also attributable to peripheral neuronal sensitization, development of pain-specific treatments would greatly benefit from the identification of novel targets specifically expressed in pain pathways, especially those targets on nociceptive primary sensory neurons. One potential target is the Mas-related G protein-coupled receptor (MRGPR). MRGPRs comprise a family of orphan G protein-coupled receptors (GPCRs) and include many genes in humans and rodents, but their physiological functions are only partially known. However, the functional properties of MRGPRX1 cannot be fully inferred from MRGPRC owing to cross-species variation in MRGPR agonist activity and receptor function. Although mouse MrgprC shares sequence homology with human MRGPRX1, it is becoming clear that human MRGPRX1 has binding and pharmacological profiles distinct from the binding and pharmacological profiles of rodent MRGPRC. For example, although the sequence of bovine adrenal medulla (BAM) peptide is conserved from rodents to human (e.g., BAM8-22 activates both MRGPRX1 and MRGPRC), most MRGPRX1-selective agonists have weak or no agonist activity at MRGPRC and do not affect rodent pain behavior. Again, such species difference is a long-lasting barrier for developing a new pain-killer drug. One can consider human dorsal root ganglia (DRG) tissue can be an alternative, however, it is difficult to isolate from donors at regular basis and the quantity of human DRG is also very low.

Human pluripotent stem cells (hPSCs), including human embryonic stem cells (hESCs) and human induced pluripotent stem cells (biPSCs), have emerged as a complementary system to animal models, because the directed specification of hESCs/hiPSCs is especially useful for generating certain cell types; they produce large quantities of otherwise extremely rare cell populations such as human nociceptive and pruriceptive neurons. Previous studies have shown that the sensory neuron population can be readily generated from hESCs/hiPSCs, however, the resulting sensory neurons are highly heterogeneous and the subtypes of sensory neurons were not sufficiently defined, purified yet or characterized in detail. Therefore, it is important to study the functions of human sensory neurons in genetically defined neuronal subsets. Provided here are methods of producing hPSC-derived TRPV1::GFP+, MRGPRX1::GFP+ and SCN9A::GFP+ sensory neurons. These methods can include functional characterization and in vivo transplantation studies.

The disclosure provided herein can help build a foundation to develop novel drug candidates as well as a potential cell therapy strategy for managing acute pain, chronic pain, and itch conditions.

### Pluripotent Stem Cells

Provided herein are populations of stem cells, pluripotent stems cells (PSCs) and pluripotent stem cell-derived sensory neurons (PSC-SNs) and human pluripotent stem cell-derived nociceptive neurons (PSC-NNs). In some embodiments, the populations of stem cells are isolated. In some embodiments, the isolated populations of cells comprise a biomarker or nociceptor marker. A biomarker or nociceptor marker of the isolated populations of cells can be determined via transcriptomic and functional profiling.

In some embodiments, the isolated population of stem cells comprises totipotent, pluripotent, multipotent, oligopotent, and unipotent stem cells. In some embodiments, the isolated population of stems cells comprises embryonic stem cells (ESCs), induced pluripotent stem cells (iPSCs), epiblast stem cells (EpiSCs), nuclear transfer embryonic stem cells (ntESCs), and embryonic germ cells (EGCs). In some embodiments, the isolated population can include one or more of nociceptors, mechanoreceptors, proprioceptors, thermoreceptors, and pruriceptors.

In some embodiments, the isolated population of pluripotent stem cells are derived from human tissue. In some embodiments, the isolated population of pluripotent stem cells are human pluripotent stem cells. In some embodiments, the isolated population of pluripotent stem cells are derived from non-human tissue. In some embodiments, the pluripotent stem cells are derived from tissue comprising bone marrow, adipose tissue, umbilical cord blood, placenta, amniotic fluid, dental pulp, peripheral blood, skin, muscle, embryos, or combination thereof. In some embodiments, the isolated population of pluripotent stem cells are allogenic pluripotent stem cells. In some embodiments, the isolated population of pluripotent stem cells are autologous pluripotent stem cells.

In some embodiments, the isolated population of cells comprise a nociceptor marker. In some embodiments, the population of cells has a high expression of the nociceptor marker. In some embodiments, at least 40%, at least 45%, at least 50%, at least 55%, at least 60%, at least 65%, at least 70%, at least 75%, at least 80%, at least 85%, at least 90%, at least 95%, at least 99% or 100% of the isolated population of cells have high expression of the nociceptor marker. In some embodiments, the isolated population of cells comprise a biomarker. In some embodiments, the population of cells has a high expression of the biomarker. In some embodiments, at least 40%, at least 45%, at least 50%, at least 55%, at least 60%, at least 65%, at least 70%, at least 75%, at least 80%, at least 85%, at least 90%, at least 95%, at least 99% or 100% of the isolated population of cells have high expression of the biomarker.

In some embodiments, the isolated population of cells comprise CD200 as a nociceptor marker. In some embodiments, the population of cells has a high expression of CD200 (e.g., referred CD200^{high} PSC-NNs). In some embodiments, at least 40%, at least 45%, at least 50%, at least 55%, at least 60%, at least 65%, at least 70%, at least 75%, at least 80%, at least 85%, at least 90%, at least 95%, at least 99% or 100% of the isolated population of cells are CD200^{high} In some embodiments, the population of PSC-NNs has a high expression of CD200. The CD200^{high} PSC-NNs can act as biological decoys by sequestering inflammatory ligands while secreting reparative factors in human and mouse joint tissues.

In some embodiments, the cells express TRPV1, SCN9A, MRGPRX1, or KCNQ2. In some embodiments, the isolated population of cells express TRPV1, SCN9A, and MRGPRX1. In some embodiments, the isolated population of cells further express TRPV1 and SCN9A nociceptors. In some embodiments, the isolated population of cells are enriched in TRPV1+ and SCN9A+ nociceptors.

An exemplary method of isolating PSC-NNs is described below. First, reporter lines expressing fluorescent tags can be generated under the control of TRPV1, SCN9A, and MRGPRX1 loci. While these reporter lines can be indispensable for in-depth transcriptomic, morphological, and functional characterization, their translational utility may be limited by concerns over the immunogenicity and cytotoxicity of fluorescent proteins. To address this, CD200 can be identified as a nociceptor-specific surface marker enriched in TRPV1+ and SCN9A+ nociceptors. CD200 can enable purification of nociceptors from heterogeneous PSC-SN populations without the need for genetic labeling. In an exemplary application, CD200^{high} hPSC-NNs can exhibit strong responses to inflammatory mediators present in synovial fluid from osteoarthritis patients and were transcriptionally enriched for nociceptive ion channels, receptors, and pathways essential for known and unknown nociceptive signaling. Additionally, NGF, a neurotrophic factor commonly used to direct sensory neuron lineage commitment, can sensitize hPSC-NNs in vitro by activating TrkA and promoting long-term expression of pronociceptive genes such as those for TRPV1, Nav1.8, and substance P 40. Therefore, the intrinsic responsiveness of hPSC-NNs to inflammatory ligands can be leveraged to sequester them within the osteoarthritis joints (e.g., knee joints).

Another exemplary application of the isolated population of cells is below. The isolated population of CD200^{high} cells can be injected into the joint of a subject and can serve as scavengers of pro-inflammatory mediators. These cells can alleviate joint pain (e.g., osteroarthritis) and modulate the endogenous neuro-immune interface by reducing aberrant nerve growth and suppressing pro-inflammatory signaling. CD200^{high} cells (e.g., CD200^{high} PSC-NNs) can be used as a therapeutic modality capable of intercepting inflammatory cues and modulating joint pathophysiology at multiple levels.

### Modified Pluripotent Stem Cells

In some embodiments, the present disclosure provides modified stem cells or pluripotent stem cells. Herein, the term “modified pluripotent stem cells” encompasses pluripotent stem cells comprising one or more genomic modifications resulting in the reduced expression and/or function of one or more endogenous target genes as well as pluripotent stem cells comprising a gene-regulating system capable of reducing the expression and/or function of one or more endogenous target genes. Herein, an “un-modified pluripotent stem cell” or “control pluripotent stem cell” refers to a cell or population of cells wherein the genomes have not been modified and that does not comprise a gene-regulating system or comprises a control gene-regulating system (e.g., an empty vector control, a non-targeting gRNA, a scrambled siRNA, etc.).

The term “pluripotent stem cell” includes but is not limited to embryonic stem cells (ESCs), induced pluripotent stem cells (iPSCs), epiblast stem cells (EpiSCs), nuclear transfer embryonic stem cells (ntESCs), and embryonic germ cells (EGCs). In some embodiments, the pluripotent stem cells are allogenic or autologous.

In some embodiments, the pluripotent stem cell is an animal cell or is derived from an animal cell, including from an invertebrate animal or a vertebrate animals (e.g., fish, amphibian, reptile, bird, or mammal). In some embodiments, the pluripotent stem cell is a mammalian cell or is derived from a mammalian cell (e.g., a pig, a cow, a goat, a sheep, a rodent, a non-human primate, a human, etc.). In some embodiments, the pluripotent stem cell is a rodent cell or is derived from a rodent cell (e.g., a rat or a mouse). In some embodiments, the modified pluripotent stem cell is a human cell or is derived from a human cell.

In some embodiments, the modified pluripotent stem cells comprise one or more modifications (e.g., insertions, deletions, or mutations of one or more nucleic acids) in the genomic DNA sequence of an endogenous target gene resulting in the reduced expression and/or function the endogenous gene. Such modifications are referred to herein as “inactivating mutations” and endogenous genes comprising an inactivating mutation are referred to as “modified endogenous target genes.” In some embodiments, the inactivating mutations reduce or inhibit mRNA transcription, thereby reducing the expression level of the encoded mRNA transcript and protein. In some embodiments, the inactivating mutations reduce or inhibit mRNA translation, thereby reducing the expression level of the encoded protein. In some embodiments, the inactivating mutations encode a modified endogenous protein with reduced or altered function compared to the unmodified (i.e., wild-type) version of the endogenous protein (e.g., a dominant-negative mutant, described infra).

In some embodiments, the modified pluripotent stem cells comprise one or more genomic modifications at a genomic location other than an endogenous target gene that result in the reduced expression and/or function of the endogenous target gene or that result in the expression of a modified version of an endogenous protein. For example, in some embodiments, a polynucleotide sequence encoding a gene regulating system is inserted into one or more locations in the genome, thereby reducing the expression and/or function of an endogenous target gene upon the expression of the gene-regulating system. In some embodiments, a polynucleotide sequence encoding a modified version of an endogenous protein is inserted at one or more locations in the genome, wherein the function of the modified version of the protein is reduced compared to the un-modified or wild-type version of the protein (e.g., a dominant-negative mutant, described infra).

In some embodiments, the modified pluripotent stem cells described herein comprises one or more modified endogenous target genes, wherein the one or more modifications result in a reduced expression and/or function of a gene product (i.e., an mRNA transcript or a protein) encoded by the endogenous target gene compared to an unmodified pluripotent stem cell. For example, in some embodiments, a modified pluripotent stem cell demonstrates reduced expression of an mRNA transcript and/or reduced expression of a protein. In some embodiments, the expression of the gene product in a modified pluripotent stem cell is reduced by at least 5% compared to the expression of the gene product in an unmodified pluripotent stem cell. In some embodiments, the expression of the gene product in a modified pluripotent stem cell is reduced by at least 10%, 20%, 30%, 40%, 50%, 60%, 70%, 80%, 90%, or more compared to the expression of the gene product in an unmodified pluripotent stem cell. In some embodiments, the modified pluripotent stem cells described herein demonstrate reduced expression and/or function of gene products encoded by a plurality (e.g., two or more) of endogenous target genes compared to the expression of the gene products in an unmodified pluripotent stem cell. For example, in some embodiments, a modified pluripotent stem cell demonstrates reduced expression and/or function of gene products from 2, 3, 4, 5, 6, 7, 8, 9, 10, or more endogenous target genes compared to the expression of the gene products in an unmodified pluripotent stem cell.

In some embodiments, the present disclosure provides a modified pluripotent stem cell wherein one or more endogenous target genes, or a portion thereof, are deleted (i.e., “knocked-out”) such that the modified pluripotent stem cell does not express the mRNA transcript or protein. In some embodiments, a modified pluripotent stem cell comprises deletion of a plurality of endogenous target genes, or portions thereof. In some embodiments, a modified pluripotent stem cell comprises deletion of 2, 3, 4, 5, 6, 7, 8, 9, 10, or more endogenous target genes.

In some embodiments, the modified pluripotent stem cells described herein comprise one or more modified endogenous target genes, wherein the one or more modifications to the target DNA sequence result in expression of a protein with reduced or altered function (e.g., a “modified endogenous protein”) compared to the function of the corresponding protein expressed in an unmodified pluripotent stem cell (e.g., a “unmodified endogenous protein”). In some embodiments, the modified pluripotent stem cells described herein comprise 2, 3, 4, 5, 6, 7, 8, 9, 10, or more modified endogenous target genes encoding 2, 3, 4, 5, 6, 7, 8, 9, 10, or more modified endogenous proteins. In some embodiments, the modified endogenous protein demonstrates reduced or altered binding affinity for another protein expressed by the modified pluripotent stem cell or expressed by another cell; reduced or altered signaling capacity; reduced or altered enzymatic activity; reduced or altered DNA-binding activity; or reduced or altered ability to function as a scaffolding protein.

In some embodiments, the modified endogenous target gene comprises one or more dominant negative mutations. As used herein, a “dominant-negative mutation” refers to a substitution, deletion, or insertion of one or more nucleotides of a target gene such that the encoded protein acts antagonistically to the protein encoded by the unmodified target gene. The mutation is dominant-negative because the negative phenotype confers genic dominance over the positive phenotype of the corresponding unmodified gene. A gene comprising one or more dominant-negative mutations and the protein encoded thereby are referred to as a “dominant-negative mutants”, e.g. dominant-negative genes and dominant-negative proteins. In some embodiments, the dominant negative mutant protein is encoded by an exogenous transgene inserted at one or more locations in the genome of the pluripotent stem cell.

Various mechanisms for dominant negativity are known. Typically, the gene product of a dominant negative mutant retains some functions of the unmodified gene product but lacks one or more crucial other functions of the unmodified gene product. This causes the dominant-negative mutant to antagonize the unmodified gene product. For example, as an illustrative embodiment, a dominant-negative mutant of a transcription factor may lack a functional activation domain but retain a functional DNA binding domain. In this example, the dominant-negative transcription factor cannot activate transcription of the DNA as the unmodified transcription factor does, but the dominant-negative transcription factor can indirectly inhibit gene expression by preventing the unmodified transcription factor from binding to the transcription-factor binding site. As another illustrative embodiment, dominant-negative mutations of proteins that function as dimers are known. Dominant-negative mutants of such dimeric proteins may retain the ability to dimerize with unmodified protein but be unable to function otherwise. The dominant-negative monomers, by dimerizing with unmodified monomers to form heterodimers, prevent formation of functional homodimers of the unmodified monomers.

In some embodiments, the modified pluripotent stem cells comprise a gene-regulating system capable of reducing the expression or function of one or more endogenous target genes. The gene-regulating system can reduce the expression and/or function of the endogenous target genes modifications by a variety of mechanisms including by modifying the genomic DNA sequence of the endogenous target gene (e.g., by insertion, deletion, or mutation of one or more nucleic acids in the genomic DNA sequence); by regulating transcription of the endogenous target gene (e.g., inhibition or repression of mRNA transcription); and/or by regulating translation of the endogenous target gene (e.g., by mRNA degradation).

In some embodiments, the modified pluripotent stem cells described herein comprise a gene-regulating system (e.g., a nucleic acid-based gene-regulating system, a protein-based gene-regulating system, or a combination protein/nucleic acid-based gene-regulating system). In such embodiments, the gene-regulating system comprised in the modified pluripotent stem cell is capable of modifying one or more endogenous target genes. In some embodiments, the modified pluripotent stem cells described herein comprise a gene-regulating system comprising: one or more nucleic acid molecules capable of reducing the expression or modifying the function of a gene product encoded by one or more endogenous target genes; one or more polynucleotides encoding a nucleic acid molecule that is capable of reducing the expression or modifying the function of a gene product encoded by one or more endogenous target genes; one or more proteins capable of reducing the expression or modifying the function of a gene product encoded by one or more endogenous target genes; one or more polynucleotides encoding a protein that is capable of reducing the expression or modifying the function of a gene product encoded by one or more endogenous target genes; one or more guide RNAs (gRNAs) capable of binding to a target DNA sequence in an endogenous gene; one or more polynucleotides encoding one or more gRNAs capable of binding to a target DNA sequence in an endogenous gene; one or more site-directed modifying polypeptides capable of interacting with a gRNA and modifying a target DNA sequence in an endogenous gene; one or more polynucleotides encoding a site-directed modifying polypeptide capable of interacting with a gRNA and modifying a target DNA sequence in an endogenous gene; one or more guide DNAs (gDNAs) capable of binding to a target DNA sequence in an endogenous gene; one or more polynucleotides encoding one or more gDNAs capable of binding to a target DNA sequence in an endogenous gene; one or more site-directed modifying polypeptides capable of interacting with a gDNA and modifying a target DNA sequence in an endogenous gene; one or more polynucleotides encoding a site-directed modifying polypeptide capable of interacting with a gDNA and modifying a target DNA sequence in an endogenous gene; one or more gRNAs capable of binding to a target mRNA sequence encoded by an endogenous gene; one or more polynucleotides encoding one or more gRNAs capable of binding to a target mRNA sequence encoded by an endogenous gene; one or more site-directed modifying polypeptides capable of interacting with a gRNA and modifying a target mRNA sequence encoded by an endogenous gene; one or more polynucleotides encoding a site-directed modifying polypeptide capable of interacting with a gRNA and modifying a target mRNA sequence encoded by an endogenous gene; or any combination of the above.

In some embodiments, one or more polynucleotides encoding the gene-regulating system are inserted into the genome of the pluripotent stem cell. In some embodiments, one or more polynucleotides encoding the gene-regulating system are expressed episomaly and are not inserted into the genome of the pluripotent stem cell.

In some embodiments, the modified pluripotent stem cells described herein comprise reduced expression and/or function of one or more endogenous target genes and further comprise one or more exogenous transgenes inserted at one or more genomic loci (e.g., a genetic “knock-in”). In some embodiments, the one or more exogenous transgenes encode detectable tags, safety-switch systems, chimeric switch receptors, and/or engineered antigen-specific receptors.

In some embodiments, the modified pluripotent stem cells described herein further comprise an exogenous transgene encoding a detectable tag. Examples of detectable tags include but are not limited to, FLAG tags, poly-histidine tags (e.g. 6×His), SNAP tags, Halo tags, cMyc tags, glutathione-S-transferase tags, avidin, enzymes, fluorescent proteins, luminescent proteins, chemiluminescent proteins, bioluminescent proteins, and phosphorescent proteins. In some embodiments the fluorescent protein is selected from the group consisting of blue/UV proteins (such as BFP, TagBFP, mTagBFP2, Azurite, EBFP2, mKalama1, Sirius, Sapphire, and T-Sapphire); cyan proteins (such as CFP, eCFP, Cerulean, SCFP3A, mTurquoise, mTurquoise2, monomeric Midoriishi-Cyan, TagCFP, and mTFP1); green proteins (such as: GFP, eGFP, meGFP (A208K mutation), Emerald, Superfolder GFP, Monomeric Azami Green, TagGFP2, mUKG, mWasabi, Clover, and mNeonGreen); yellow proteins (such as YFP, eYFP, Citrine, Venus, SYFP2, and TagYFP); orange proteins (such as Monomeric Kusabira-Orange, mKOx, mKO2, mOrange, and mOrange2); red proteins (such as RFP, mRaspberry, mCherry, mStrawberry, mTangerine, tdTomato, TagRFP, TagRFP-T, mApple, mRuby, and mRuby2); far-red proteins (such as mPlum, HcRed-Tandem, mKate2, mNeptune, and NirFP); near-infrared proteins (such as TagRFP657, IFP1.4, and iRFP); long stokes shift proteins (such as mKeima Red, LSS-mKate1, LSS-mKate2, and mBeRFP); photoactivatible proteins (such as PA-GFP, PAmCherry1, and PATagRFP); photoconvertible proteins (such as Kaede (green), Kaede (red), KikGR1 (green), KikGR1 (red), PS-CFP2, PS-CFP2, mEos2 (green), mEos2 (red), mEos3.2 (green), mEos3.2 (red), PSmOrange, and PSmOrange); and photoswitchable proteins (such as Dronpa). In some embodiments, the detectable tag can be selected from AmCyan, AsRed, DsRed2, DsRed Express, E2-Crimson, HcRed, ZsGreen, ZsYellow, mCherry, mStrawberry, mOrange, mBanana, mPlum, mRasberry, tdTomato, DsRed Monomer, and/or AcGFP, all of which are available from Clontech.

In some embodiments, the modified pluripotent stem cells described herein further comprise an exogenous transgene encoding a safety-switch system. Safety-switch systems (also referred to in the art as suicide gene systems) comprise exogenous transgenes encoding for one or more proteins that enable the elimination of a modified pluripotent stem cell after the cell has been administered to a subject. Examples of safety-switch systems are known in the art. For example, safety-switch systems include genes encoding for proteins that convert non-toxic pro-drugs into toxic compounds such as the Herpes simplex thymidine kinase (Hsv-tk) and ganciclovir (GCV) system (Hsv-tk/GCV). Hsv-tk converts non-toxic GCV into a cytotoxic compound that leads to cellular apoptosis. As such, administration of GCV to a subject that has been treated with modified pluripotent stem cells comprising a transgene encoding the Hsv-tk protein can selectively eliminate the modified pluripotent stem cells while sparing endogenous pluripotent stem cells. (See e.g., Bonini et al., Science, 1997, 276(5319):1719-1724; Ciceri et al., Blood, 2007, 109(11):1828-1836; Bondanza et al., Blood 2006, 107(5):1828-1836).

Additional safety-switch systems include genes encoding for cell-surface markers, enabling elimination of modified pluripotent stem cells by administration of a monoclonal antibody specific for the cell-surface marker via ADCC. In some embodiments, the modified pluripotent stem cells described herein further comprise an exogenous transgene encoding a chimeric switch receptor. Chimeric switch receptors are engineered cell-surface receptors comprising an extracellular domain from an endogenous cell-surface receptor and a heterologous intracellular signaling domain, such that ligand recognition by the extracellular domain results in activation of a different signaling cascade than that activated by the wild type form of the cell-surface receptor. In some embodiments, the chimeric switch receptor comprises the extracellular domain of an inhibitory cell-surface receptor fused to an intracellular domain that leads to the transmission of an activating signal rather than the inhibitory signal normally transduced by the inhibitory cell-surface receptor. In particular embodiments, extracellular domains derived from cell-surface receptors known to inhibit pluripotent stem cell activation can be fused to activating intracellular domains. Engagement of the corresponding ligand will then activate signaling cascades that increase, rather than inhibit, the activation of the pluripotent stem cell.

### Gene-Regulating Systems

Herein, the term “gene-regulating system” refers to a protein, nucleic acid, or combination thereof that is capable of modifying an endogenous target DNA sequence when introduced into a cell, thereby regulating the expression or function of the encoded gene product. Numerous gene editing systems suitable for use in the methods of the present disclosure are known in the art including, but not limited to, shRNAs, siRNAs, zinc-finger nuclease systems, TALEN systems, and CRISPR/Cas systems.

As used herein, “regulate,” when used in reference to the effect of a gene-regulating system on an endogenous target gene encompasses any change in the sequence of the endogenous target gene, any change in the epigenetic state of the endogenous target gene, and/or any change in the expression or function of the protein encoded by the endogenous target gene.

In some embodiments, the gene-regulating system may mediate a change in the sequence of the endogenous target gene, for example, by introducing one or more mutations into the endogenous target sequence, such as by insertion or deletion of one or more nucleic acids in the endogenous target sequence. Exemplary mechanisms that can mediate alterations of the endogenous target sequence include, but are not limited to, non-homologous end joining (NHEJ) (e.g., classical or alternative), microhomology-mediated end joining (NMMEJ), homology-directed repair (e.g., endogenous donor template mediated), SDSA (synthesis dependent strand annealing), single strand annealing or single strand invasion.

In some embodiments, the gene-regulating system may mediate a change in the epigenetic state of the endogenous target sequence. For example, in some embodiments, the gene-regulating system may mediate covalent modifications of the endogenous target gene DNA (e.g., cytosine methylation and hydroxymethylation) or of associated histone proteins (e.g. lysine acetylation, lysine and arginine methylation, serine and threonine phosphorylation, and lysine ubiquitination and sumoylation).

In some embodiments, the gene-regulating system may mediate a change in the expression of the protein encoded by the endogenous target gene. In such embodiments, the gene-regulating system may regulate the expression of the encoded protein by modifications of the endogenous target DNA sequence, or by acting on the mRNA product encoded by the DNA sequence. In some embodiments, the gene-regulating system may result in the expression of a modified endogenous protein. In such embodiments, the modifications to the endogenous DNA sequence mediated by the gene-regulating system result in the expression of an endogenous protein demonstrating a reduced function as compared to the corresponding endogenous protein in an unmodified pluripotent stem cell. In such embodiments, the expression level of the modified endogenous protein may be increased, decreased or may be the same, or substantially similar to, the expression level of the corresponding endogenous protein in an unmodified pluripotent stem cell.

### Combination of Nucleic Acid/Protein-Based Gene-Regulating Systems

Combination gene-regulating systems comprise a site-directed modifying polypeptide and a nucleic acid guide molecule. Herein, a “site-directed modifying polypeptide” refers to a polypeptide that binds to a nucleic acid guide molecule, is targeted to a target nucleic acid sequence, (for example, an endogenous target DNA or RNA sequence) by the nucleic acid guide molecule to which it is bound, and modifies the target nucleic acid sequence (e.g., by cleavage, mutation, or methylation of the target nucleic acid sequence).

A site-directed modifying polypeptide comprises two portions, a portion that binds the nucleic acid guide and an activity portion. In some embodiments, a site-directed modifying polypeptide comprises an activity portion that exhibits site-directed enzymatic activity (e.g., DNA methylation, DNA or RNA cleavage, histone acetylation, histone methylation, etc.), wherein the site of enzymatic activity is determined by the guide nucleic acid. In some cases, a site-directed modifying polypeptide comprises an activity portion that has enzymatic activity that modifies the endogenous target nucleic acid sequence (e.g., nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity). In other cases, a site-directed modifying polypeptide comprises an activity portion that has enzymatic activity that modifies a polypeptide (e.g., a histone) associated with the endogenous target nucleic acid sequence (e.g., methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity). In some embodiments, a site-directed modifying polypeptide comprises an activity portion that modulates transcription of a target DNA sequence (e.g., to increase or decrease transcription). In some embodiments, a site-directed modifying polypeptide comprises an activity portion that modulates expression or translation of a target RNA sequence (e.g., to increase or decrease transcription).

The nucleic acid guide comprises two portions: a first portion that is complementary to, and capable of binding with, an endogenous target nucleic sequence (referred to herein as a “nucleic acid-binding segment”), and a second portion that is capable of interacting with the site-directed modifying polypeptide (referred to herein as a “protein-binding segment”). In some embodiments, the nucleic acid-binding segment and protein-binding segment of a nucleic acid guide are comprised within a single polynucleotide molecule. In some embodiments, the nucleic acid-binding segment and protein-binding segment of a nucleic acid guide are each comprised within separate polynucleotide molecules, such that the nucleic acid guide comprises two polynucleotide molecules that associate with each other to form the functional guide.

The nucleic acid guide mediates the target specificity of the combined protein/nucleic acid gene-regulating systems by specifically hybridizing with a target nucleic acid sequence. In some embodiments, the target nucleic acid sequence is an RNA sequence, such as an RNA sequence comprised within an mRNA transcript of a target gene. In some embodiments, the target nucleic acid sequence is a DNA sequence comprised within the DNA sequence of a target gene. Reference herein to a target gene encompasses the full-length DNA sequence for that particular gene which comprises a plurality of target genetic loci (i.e., portions of a particular target gene sequence (e.g., an exon or an intron)). Within each target genetic loci are shorter stretches of DNA sequences referred to herein as “target DNA sequences” that can be modified by the gene-regulating systems described herein. Further, each target genetic loci comprises a “target modification site,” which refers to the precise location of the modification induced by the gene-regulating system (e.g., the location of an insertion, a deletion, or mutation, the location of a DNA break, or the location of an epigenetic modification).

The gene-regulating systems described herein may comprise a single nucleic acid guide, or may comprise a plurality of nucleic acid guides (e.g., 2, 3, 4, 5, 6, 7, 8, 9, 10, or more nucleic acid guides).

In some embodiments, the combined protein/nucleic acid gene-regulating systems comprise site-directed modifying polypeptides derived from Argonaute (Ago) proteins (e.g., T. thermophiles Ago or TtAgo). In such embodiments, the site-directed modifying polypeptide is a T. thermophiles Ago DNA endonuclease and the nucleic acid guide is a guide DNA (gDNA) (See, Swarts et al., Nature 507 (2014), 258-261). In some embodiments, the present disclosure provides a polynucleotide encoding a gDNA. In some embodiments, a gDNA-encoding nucleic acid is comprised in an expression vector, e.g., a recombinant expression vector. In some embodiments, the present disclosure provides a polynucleotide encoding a TtAgo site-directed modifying polypeptide or variant thereof. In some embodiments, the polynucleotide encoding a TtAgo site-directed modifying polypeptide is comprised in an expression vector, e.g., a recombinant expression vector.

In some embodiments, the gene editing systems described herein are CRISPR (Clustered Regularly Interspaced Short Palindromic Repeats)/Cas (CRISPR Associated) nuclease systems. In some embodiments, the CRISPR/Cas system is a Class 2 system. Class 2 CRISPR/Cas systems are divided into three types: Type II, Type V, and Type VI systems. In some embodiments, the CRISPR/Cas system is a Class 2 Type II system, utilizing the Cas9 protein. In such embodiments, the site-directed modifying polypeptide is a Cas9 DNA endonuclease (or variant thereof) and the nucleic acid guide molecule is a guide RNA (gRNA). In some embodiments, the CRISPR/Cas system is a Class 2 Type V system, utilizing the Cas12 proteins (e.g., Cas12a (also known as Cpf1), Cas12b (also known as C2c1), Cas12c (also known as C2c3), Cas12d (also known as CasY), and Cas12e (also known as CasX)). In such embodiments, the site-directed modifying polypeptide is a Cas12 DNA endonuclease (or variant thereof) and the nucleic acid guide molecule is a gRNA. In some embodiments, the CRISPR/Cas system is a Class 2 and Type VI system, utilizing the Cas13 proteins (e.g., Cas13a (also known as C2c2), Cas13b, and Cas13c). (See, Pyzocha et al., ACS Chemical Biology, 13(2), 347-356). In such embodiments, the site-directed modifying polypeptide is a Cas13 RNA riboendonuclease and the nucleic acid guide molecule is a gRNA.

A Cas polypeptide refers to a polypeptide that can interact with a gRNA molecule and, in concert with the gRNA molecule, home or localize to a target DNA or target RNA sequence. Cas polypeptides include naturally occurring Cas proteins and engineered, altered, or otherwise modified Cas proteins that differ by one or more amino acid residues from a naturally-occurring Cas sequence.

A guide RNA (gRNA) comprises two segments, a DNA-binding segment and a protein-binding segment. In some embodiments, the protein-binding segment of a gRNA is comprised in one RNA molecule and the DNA-binding segment is comprised in another separate RNA molecule. Such embodiments are referred to herein as “double-molecule gRNAs” or “two-molecule gRNA” or “dual gRNAs.” In some embodiments, the gRNA is a single RNA molecule and is referred to herein as a “single-guide RNA” or an “sgRNA.” The term “guide RNA” or “gRNA” is inclusive, referring both to two-molecule guide RNAs and sgRNAs.

The protein-binding segment of a gRNA comprises, in part, two complementary stretches of nucleotides that hybridize to one another to form a double stranded RNA duplex (dsRNA duplex), which facilitates binding to the Cas protein. The nucleic acid-binding segment (or “nucleic acid-binding sequence”) of a gRNA comprises a nucleotide sequence that is complementary to and capable of binding to a specific target nucleic acid sequence. The protein-binding segment of the gRNA interacts with a Cas polypeptide and the interaction of the gRNA molecule and site-directed modifying polypeptide results in Cas binding to the endogenous nucleic acid sequence and produces one or more modifications within or around the target nucleic acid sequence. The precise location of the target modification site is determined by both (i) base-pairing complementarity between the gRNA and the target nucleic acid sequence; and (ii) the location of a short motif, referred to as the protospacer adjacent motif (PAM), in the target DNA sequence (referred to as a protospacer flanking sequence (PFS) in target RNA sequences). The PAM/PFS sequence is required for Cas binding to the target nucleic acid sequence. A variety of PAM/PFS sequences are known in the art and are suitable for use with a particular Cas endonuclease (e.g., a Cas9 endonuclease)(See e.g., Nat Methods. 2013 November; 10(11): 1116-1121 and Sci Rep. 2014; 4: 5405). In some embodiments, the PAM sequence is located within 50 base pairs of the target modification site in a target DNA sequence. In some embodiments, the PAM sequence is located within 10 base pairs of the target modification site in a target DNA sequence. The DNA sequences that can be targeted by this method are limited only by the relative distance of the PAM sequence to the target modification site and the presence of a unique 20 base pair sequence to mediate sequence-specific, gRNA-mediated Cas binding. In some embodiments, the PFS sequence is located at the 3′ end of the target RNA sequence. In some embodiments, the target modification site is located at the 5′ terminus of the target locus. In some embodiments, the target modification site is located at the 3′ end of the target locus. In some embodiments, the target modification site is located within an intron or an exon of the target locus.

In some embodiments, the PAM sequence comprises a nucleic acid sequence having at least 90% or 95% identity to any one of the sequences in Tables 1 or 2. In some embodiments, the PAM sequence comprises a nucleic acid sequence identical to any one of the sequences in Tables 1 or 2.

In some embodiments, the present disclosure provides a polynucleotide encoding a gRNA. In some embodiments, a gRNA-encoding nucleic acid is comprised in an expression vector, e.g., a recombinant expression vector. In some embodiments, the present disclosure provides a polynucleotide encoding a site-directed modifying polypeptide. In some embodiments, the polynucleotide encoding a site-directed modifying polypeptide is comprised in an expression vector, e.g., a recombinant expression vector.

### 1. Cas Proteins

In some embodiments, the site-directed modifying polypeptide is a Cas protein. Cas molecules of a variety of species can be used in the methods and compositions described herein, including Cas molecules derived from S. pyogenes, S. aureus, N. meningitidis, S. thermophiles, Acidovorax avenae, Actinobacillus pleuropneumoniae, Actinobacillus succinogenes, Actinobacillus suis, Actinomyces sp., Cycliphilus denitrificans, Aminomonas paucivorans, Bacillus cereus, Bacillus smithii, Bacillus thuringiensis, Bacteroides sp., Blastopirellula marina, Bradyrhizobium sp., Brevibacillus laterospoxus, Campylobacter coli, Campylobacter jejuni, Campylobacter lari, Candidatus puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, Gamma proteobacterium, Gluconacetobacter diazotrophicus, Haemophilus parainfluenzae, Haemophilus sputomm, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria meningitidis, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus aureus, Staphylococcus lugdunensis, Streptococcus sp., Subdoligranulum sp., Tistrella mobilis, Treponema sp., or Verminephrobacter eiseniae.

In some embodiments, the Cas protein is a naturally-occurring Cas protein. In some embodiments, the Cas endonuclease is selected from the group consisting of C2C1, C2C3, Cpf1 (also referred to as Cas12a), Cas12b, Cas12c, Cas12d, Cas12e, Cas13a, Cas13b, Cas13c, Cas13d, Cas1, CaslB, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4.

In some embodiments, the Cas protein is an endoribonuclease such as a Cas13 protein. In some embodiments, the Cas13 protein is a Cas13a (Abudayyeh et al., Nature 550 (2017), 280-284), Cas13b (Cox et al., Science (2017) 358:6336, 1019-1027), Cas13c (Cox et al., Science (2017) 358:6336, 1019-1027), or Cas13d (Zhang et al., Cell 175 (2018), 212-223) protein.

In some embodiments, the Cas protein is a wild-type or naturally occurring Cas9 protein or a Cas9 ortholog. Wild-type Cas9 is a multi-domain enzyme that uses an HNH nuclease domain to cleave the target strand of DNA and a RuvC-like domain to cleave the non-target strand. Binding of WT Cas9 to DNA based on gRNA specificity results in double-stranded DNA breaks that can be repaired by non-homologous end joining (NHEJ) or homology-directed repair (HDR). Exemplary naturally occurring Cas9 molecules are described in Chylinski et al., RNA Biology 2013 10:5, 727-737 and additional Cas9 orthologs are described in International PCT Publication No. WO 2015/071474. Such Cas9 molecules include Cas9 molecules of a cluster 1 bacterial family, cluster 2 bacterial family, cluster 3 bacterial family, cluster 4 bacterial family, cluster 5 bacterial family, cluster 6 bacterial family, a cluster 7 bacterial family, a cluster 8 bacterial family, a cluster 9 bacterial family, a cluster 10 bacterial family, a cluster 1 1 bacterial family, a cluster 12 bacterial family, a cluster 13 bacterial family, a cluster 14 bacterial family, a cluster 15 bacterial family, a cluster 16 bacterial family, a cluster 17 bacterial family, a cluster 18 bacterial family, a cluster 19 bacterial family, a cluster 20 bacterial family, a cluster 21 bacterial family, a cluster 22 bacterial family, a cluster 23 bacterial family, a cluster 24 bacterial family, a cluster 25 bacterial family, a cluster 26 bacterial family, a cluster 27 bacterial family, a cluster 28 bacterial family, a cluster 29 bacterial family, a cluster 30 bacterial family, a cluster 31 bacterial family, a cluster 32 bacterial family, a cluster 33 bacterial family, a cluster 34 bacterial family, a cluster 35 bacterial family, a cluster 36 bacterial family, a cluster 37 bacterial family, a cluster 38 bacterial family, a cluster 39 bacterial family, a cluster 40 bacterial family, a cluster 41 bacterial family, a cluster 42 bacterial family, a cluster 43 bacterial family, a cluster 44 bacterial family, a cluster 45 bacterial family, a cluster 46 bacterial family, a cluster 47 bacterial family, a cluster 48 bacterial family, a cluster 49 bacterial family, a cluster 50 bacterial family, a cluster 51 bacterial family, a cluster 52 bacterial family, a cluster 53 bacterial family, a cluster 54 bacterial family, a cluster 55 bacterial family, a cluster 56 bacterial family, a cluster 57 bacterial family, a cluster 58 bacterial family, a cluster 59 bacterial family, a cluster 60 bacterial family, a cluster 61 bacterial family, a cluster 62 bacterial family, a cluster 63 bacterial family, a cluster 64 bacterial family, a cluster 65 bacterial family, a cluster 66 bacterial family, a cluster 67 bacterial family, a cluster 68 bacterial family, a cluster 69 bacterial family, a cluster 70 bacterial family, a cluster 71 bacterial family, a cluster 72 bacterial family, a cluster 73 bacterial family, a cluster 74 bacterial family, a cluster 75 bacterial family, a cluster 76 bacterial family, a cluster 77 bacterial family, or a cluster 78 bacterial family.

In some embodiments, the naturally occurring Cas9 polypeptide is selected from the group consisting of SpCas9, SpCas9-HF1, SpCas9-HF2, SpCas9-HF3, SpCas9-HF4, SaCas9, FnCpf, FnCas9, eSpCas9, and NmeCas9. In some embodiments, the Cas9 protein comprises an amino acid sequence having at least 60%, 65%, 70%, 75%, 80%, 85%, 90%, 95%, 96%, 97%, 98%, 99%, or 100% sequence identity to a Cas9 amino acid sequence described in Chylinski et al., RNA Biology 2013 10:5, 727-737; Hou et al., PNAS Early Edition 2013, 1-6).

In some embodiments, the Cas polypeptide comprises one or more of the following activities:
a. a nickase activity, i.e., the ability to cleave a single strand, e.g., the non-complementary strand or the complementary strand, of a nucleic acid molecule;
b. a double stranded nuclease activity, i.e., the ability to cleave both strands of a double stranded nucleic acid and create a double stranded break, which in an embodiment is the presence of two nickase activities;
c. an endonuclease activity;
d. an exonuclease activity; and/or
e. a helicase activity, i.e., the ability to unwind the helical structure of a double stranded nucleic acid.

In some embodiments, the Cas polypeptide is fused to heterologous proteins that recruit DNA-damage signaling proteins, exonucleases, or phosphatases to further increase the likelihood or the rate of repair of the target sequence by one repair mechanism or another. In some embodiments, a WT Cas polypeptide is co-expressed with a nucleic acid repair template to facilitate the incorporation of an exogenous nucleic acid sequence by homology-directed repair.

In some embodiments, different Cas proteins (i.e., Cas9 proteins from various species) may be advantageous to use in the various provided methods in order to capitalize on various enzymatic characteristics of the different Cas proteins (e.g., for different PAM sequence preferences; for increased or decreased enzymatic activity; for an increased or decreased level of cellular toxicity; to change the balance between NHEJ, homology-directed repair, single strand breaks, double strand breaks, etc.).

In some embodiments, the Cas protein is a Cas9 protein derived from S. pyogenes and recognizes the PAM sequence motif NGG, NAG, NGA (Mali et al, Science 2013; 339(6121): 823-826). In some embodiments, the Cas protein is a Cas9 protein derived from S. thermophiles and recognizes the PAM sequence motif NGGNG and/or NNAGAAW (W=A or T) (See, e.g., Horvath et al, Science, 2010; 327(5962): 167-170, and Deveau et al, J Bacteriol 2008; 190(4): 1390-1400). In some embodiments, the Cas protein is a Cas9 protein derived from S. mutans and recognizes the PAM sequence motif NGG and/or NAAR (R=A or G) (See, e.g., Deveau et al, J BACTERIOL 2008; 190(4): 1390-1400). In some embodiments, the Cas protein is a Cas9 protein derived from S. aureus and recognizes the PAM sequence motif NNGRR (R=A or G). In some embodiments, the Cas protein is a Cas9 protein derived from S. aureus and recognizes the PAM sequence motif N GRRT (R=A or G). In some embodiments, the Cas protein is a Cas9 protein derived from S. aureus and recognizes the PAM sequence motif N GRRV (R=A or G). In some embodiments, the Cas protein is a Cas9 protein derived from N. meningitidis and recognizes the PAM sequence motif N GATT or N GCTT (R=A or G, V=A, G or C) (See, e.g., Hou et ah, PNAS 2013, 1-6). In the aforementioned embodiments, N can be any nucleotide residue, e.g., any of A, G, C or T. In some embodiments, the Cas protein is a Cas13a protein derived from Leptotrichia shahii and recognizes the PFS sequence motif of a single 3′ A, U, or C.

In some embodiments, a polynucleotide encoding a Cas protein is provided. In some embodiments, the polynucleotide encodes a Cas protein that is at least 90% identical to a Cas protein described in International PCT Publication No. WO 2015/071474 or Chylinski et al., RNA Biology 2013 10:5, 727-737. In some embodiments, the polynucleotide encodes a Cas protein that is at least 95%, 96%, 97%, 98%, or 99% identical to a Cas protein described in International PCT Publication No. WO 2015/071474 or Chylinski et al., RNA Biology 2013 10:5, 727-737. In some embodiments, the polynucleotide encodes a Cas protein that is 100% identical to a Cas protein described in International PCT Publication No. WO 2015/071474 or Chylinski et al., RNA Biology 2013 10:5, 727-737.

### 2. Cas Mutants

In some embodiments, the Cas polypeptides are engineered to alter one or more properties of the Cas polypeptide. For example, in some embodiments, the Cas polypeptide comprises altered enzymatic properties, e.g., altered nuclease activity, (as compared with a naturally occurring or other reference Cas molecule) or altered helicase activity. In some embodiments, an engineered Cas polypeptide can have an alteration that alters its size, e.g., a deletion of amino acid sequence that reduces its size without significant effect on another property of the Cas polypeptide. In some embodiments, an engineered Cas polypeptide comprises an alteration that affects PAM recognition. For example, an engineered Cas polypeptide can be altered to recognize a PAM sequence other than the PAM sequence recognized by the corresponding wild-type Cas protein.

Cas polypeptides with desired properties can be made in a number of ways, including alteration of a naturally occurring Cas polypeptide or parental Cas polypeptide, to provide a mutant or altered Cas polypeptide having a desired property. For example, one or more mutations can be introduced into the sequence of a parental Cas polypeptide (e.g., a naturally occurring or engineered Cas polypeptide). Such mutations and differences may comprise substitutions (e.g., conservative substitutions or substitutions of non-essential amino acids); insertions; or deletions. In some embodiments, a mutant Cas polypeptide comprises one or more mutations (e.g., at least 1, 2, 3, 4, 5, 10, 15, 20, 30, 40 or 50 mutations) relative to a parental Cas polypeptide.

In an embodiment, a mutant Cas polypeptide comprises a cleavage property that differs from a naturally occurring Cas polypeptide. In some embodiments, the Cas is a deactivated Cas (dCas) mutant. In such embodiments, the Cas polypeptide does not comprise any intrinsic enzymatic activity and is unable to mediate target nucleic acid cleavage. In such embodiments, the dCas may be fused with a heterologous protein that is capable of modifying the target nucleic acid in a non-cleavage based manner. For example, in some embodiments, a dCas protein is fused to transcription activator or transcription repressor domains (e.g., the Kruppel associated box (KRAB or SKD); the Mad mSIN3 interaction domain (SID or SID4X); the ERF repressor domain (ERD); the MAX-interacting protein 1 (MXI1); methyl-CpG binding protein 2 (MECP2); etc.). In some such cases, the dCas fusion protein is targeted by the ggRNA to a specific location (i.e., sequence) in the target nucleic acid and exerts locus-specific regulation such as blocking RNA polymerase binding to a promoter (which selectively inhibits transcription activator function), and/or modifying the local chromatin status (e.g., when a fusion sequence is used that modifies the target DNA or modifies a polypeptide associated with the target DNA). In some cases, the changes are transient (e.g., transcription repression or activation). In some cases, the changes are inheritable (e.g., when epigenetic modifications are made to the target DNA or to proteins associated with the target DNA, e.g., nucleosomal histones).

In some embodiments, the dCas is a dCas13 mutant (Konermann et al., Cell 173 (2018), 665-676). These dCas13 mutants can then be fused to enzymes that modify RNA, including adenosine deaminases (e.g., ADAR1 and ADAR2). Adenosine deaminases convert adenine to inosine, which the translational machinery treats like guanine, thereby creating a functional A á G change in the RNA sequence. In some embodiments, the dCas is a dCas9 mutant.

In some embodiments, the mutant Cas9 is a Cas9 nickase mutant. Cas9 nickase mutants comprise only one catalytically active domain (either the HNH domain or the RuvC domain). The Cas9 nickase mutants retain DNA binding based on gRNA specificity, but are capable of cutting only one strand of DNA resulting in a single-strand break (e.g. a “nick”). In some embodiments, two complementary Cas9 nickase mutants (e.g., one Cas9 nickase mutant with an inactivated RuvC domain, and one Cas9 nickase mutant with an inactivated HNH domain) are expressed in the same cell with two gRNAs corresponding to two respective target sequences; one target sequence on the sense DNA strand, and one on the antisense DNA strand. This dual-nickase system results in staggered double stranded breaks and can increase target specificity, as it is unlikely that two off-target nicks will be generated close enough to generate a double stranded break. In some embodiments, a Cas9 nickase mutant is co-expressed with a nucleic acid repair template to facilitate the incorporation of an exogenous nucleic acid sequence by homology-directed repair.

In some embodiments, the Cas polypeptides described herein can be engineered to alter the PAM/PFS specificity of the Cas polypeptide. In some embodiments, a mutant Cas polypeptide has a PAM/PFS specificity that is different from the PAM/PFS specificity of the parental Cas polypeptide. For example, a naturally occurring Cas protein can be modified to alter the PAM/PFS sequence that the mutant Cas polypeptide recognizes to decrease off target sites, improve specificity, or eliminate a PAM/PFS recognition requirement. In some embodiments, a Cas protein can be modified to increase the length of the PAM/PFS recognition sequence. In some embodiments, the length of the PAM recognition sequence is at least 4, 5, 6, 7, 8, 9, 10 or 15 amino acids in length. Cas polypeptides that recognize different PAM/PFS sequences and/or have reduced off-target activity can be generated using directed evolution. Exemplary methods and systems that can be used for directed evolution of Cas polypeptides are described, e.g., in Esvelt et al. Nature 2011, 472(7344): 499-503.

Exemplary Cas mutants are described in International PCT Publication No. WO 2015/161276 and Konermann et al., Cell 173 (2018), 665-676 which are incorporated herein by reference in their entireties.

3. gRNAs

The present disclosure provides guide RNAs (gRNAs) that direct a site-directed modifying polypeptide to a specific target nucleic acid sequence. A gRNA comprises a “nucleic acid-targeting domain” or “targeting domain” and protein-binding segment. The targeting domain may also be referred to as a “spacer” sequence and comprises a nucleotide sequence that is complementary to a target nucleic acid sequence. As such, the targeting domain segment of a gRNA interacts with a target nucleic acid in a sequence-specific manner via hybridization (i.e., base pairing) and determines the location within the target nucleic acid that the gRNA will bind. The targeting domain segment of a gRNA can be modified (e.g., by genetic engineering) to hybridize to a desired sequence within a target nucleic acid sequence. In some embodiments, the targeting domain sequence is between about 13 and about 22 nucleotides in length. In some embodiments, the targeting domain sequence is about 13, 14, 15, 16, 17, 18, 19, 20, 21, or 22 nucleotides in length. In some embodiments, the targeting domain sequence is about 20 nucleotides in length.

The protein-binding segment of a gRNA interacts with a site-directed modifying polypeptide (e.g. a Cas protein) to form a ribonucleoprotein (RNP) complex comprising the gRNA and the site-directed modifying polypeptide. The targeting domain segment of the gRNA then guides the bound site-directed modifying polypeptide to a specific nucleotide sequence within target nucleic acid via the above-described spacer sequence. The protein-binding segment of a gRNA comprises at least two stretches of nucleotides that are complementary to one another and which form a double stranded RNA duplex. The protein-binding segment of a gRNA may also be referred to as a “scaffold” segment or a “tracr RNA”. In some embodiments, the tracr RNA sequence is between about 30 and about 180 nucleotides in length. In some embodiments, the tracr RNA sequence is between about 40 and about 90 nucleotides, about 50 and about 90 nucleotides, about 60 and about 90 nucleotides, about 65 and about 85 nucleotides, about 70 and about 80 nucleotides, about 65 and about 75 nucleotides, or about 75 and about 85 nucleotides in length. In some embodiments, the tracr RNA sequence is about 70, 71, 72, 73, 74, 75, 76, 77, 78, 79, 80, 81, 82, 83, 84, 85, 86, 87, 88, or about 90 nucleotides in length.

In some embodiments, a gRNA comprises two separate RNA molecules (i.e., a “dual gRNA”). In some embodiments, a gRNA comprises a single RNA molecule (i.e. a “single guide RNA” or “sgRNA”). Herein, use of the term “guide RNA” or “gRNA” is inclusive of both dual gRNAs and sgRNAs. A dual gRNA comprises two separate RNA molecules: a “crispr RNA” (or “crRNA”) and a “tracr RNA”. A crRNA molecule comprises a spacer sequence covalently linked to a “tracr mate” sequence. The tracer mate sequence comprises a stretch of nucleotides that are complementary to a corresponding sequence in the tracr RNA molecule. The crRNA molecule and tracr RNA molecule hybridize to one another via the complementarity of the tracr and tracer mate sequences.

In some embodiments, the gRNA is an sgRNA. In such embodiments, the nucleic acid-targeting sequence and the protein-binding sequence are present in a single RNA molecule by fusion of the spacer sequence to the tracr RNA sequence. In some embodiments, the sgRNA is about 50 to about 200 nucleotides in length. In some embodiments, the sgRNA is about 75 to about 150 or about 100 to about 125 nucleotides in length. In some embodiments, the sgRNA is about 100 nucleotides in length.

In some embodiments, the gRNAs of the present disclosure comprise a targeting domain sequence that is least 90%, 95%, 96%, 97%, 98%, or 99% complementary, or is 100% complementary to a target nucleic acid sequence within a target locus. In some embodiments, the target nucleic acid sequence is an RNA target sequence. In some embodiments, the target nucleic acid sequence is a DNA target sequence. In some embodiments, the target sequence comprises the nucleic acid sequence of a target gene comprising SCN9A, TRPV1, MRGPRX1, or KCNQ2. In some embodiments, the target nucleic acid sequence has at least about 80%, at least about 85%, at least about 90%, at least about 95% or about 100% identity to any one of SEQ ID NOs: 1, 3, 4, or 10. In some embodiments, the target nucleic acid sequence comprises one or more of SEQ NOs: 1, 3, 4, or 10. In some embodiments, the gRNAs provided herein comprise a targeting domain sequence that binds to a target DNA sequence that is at least 90%, 95%, 96%, 97%, 98%, or 99% identical, or is 100% identical to a sequence of a target gene selected from SCN9A, TRPV1, MRGPRX1, or KCNQ2. In some embodiments, the targeting domain sequence binds to a target DNA sequence that is at least 90%, 95%, 96%, 97%, 98%, or 99% identical, or is 100% identical to any one of SEQ ID NOs: 1, 3, 4, or 10.

In some embodiments, the gene-regulating system comprises two or more gRNA molecules. In some embodiments, the gene-regulating system comprises two or more gRNA molecules, wherein at least one of the gRNAs comprises a targeting domain that binds to a target DNA sequence that is at least 90%, 95%, 96%, 97%, 98%, or 99% identical, or is 100% identical to a target sequence of a target gene selected from SCN9A, TRPV1, MRGPRX1, or KCNQ2.

In some embodiments, the nucleic acid-binding segments of the gRNA sequences described herein are designed to minimize off-target binding using algorithms known in the art (e.g., Cas-OFF finder) to identify target sequences that are unique to a particular target locus or target gene.

In some embodiments, the gRNAs described herein can comprise one or more modified nucleosides or nucleotides which introduce stability toward nucleases. In such embodiments, these modified gRNAs may elicit a reduced innate immune as compared to a non-modified gRNA. The term “innate immune response” includes a cellular response to exogenous nucleic acids, including single stranded nucleic acids, generally of viral or bacterial origin, which involves the induction of cytokine expression and release, particularly the interferons, and cell death.

In some embodiments, the gRNAs described herein are modified at or near the 5′ end (e.g., within 1-10, 1-5, or 1-2 nucleotides of their 5′ end). In some embodiments, the 5′ end of a gRNA is modified by the inclusion of a eukaryotic mRNA cap structure or cap analog (e.g., a G(5′)ppp(5′)G cap analog, a m7G(5′)ppp(5′)G cap analog, or a 3′-O-Me-m7G(5′)ppp(5′)G anti reverse cap analog (ARCA)). In some embodiments, an in vitro transcribed gRNA is modified by treatment with a phosphatase (e.g., calf intestinal alkaline phosphatase) to remove the 5′ triphosphate group. In some embodiments, a gRNA comprises a modification at or near its 3′ end (e.g., within 1-10, 1-5, or 1-2 nucleotides of its 3′ end). For example, in some embodiments, the 3′ end of a gRNA is modified by the addition of one or more (e.g., 25-200) adenine (A) residues.

In some embodiments, modified nucleosides and modified nucleotides can be present in a gRNA, but also may be present in other gene-regulating systems, e.g., mRNA, RNAi, or siRNA-based systems. In some embodiments, modified nucleosides and nucleotides can include one or more of:
a. alteration, e.g., replacement, of one or both of the non-linking phosphate oxygens and/or of one or more of the linking phosphate oxygens in the phosphodiester backbone linkage;
b. alteration, e.g., replacement, of a constituent of the ribose sugar, e.g., of the 2′ hydroxyl on the ribose sugar;
c. wholesale replacement of the phosphate moiety with “dephospho” linkers;
d. modification or replacement of a naturally occurring nucleobase;
e. replacement or modification of the ribose-phosphate backbone;
f. modification of the 3′ end or 5′ end of the oligonucleotide, e.g., removal, modification or replacement of a terminal phosphate group or conjugation of a moiety; and
g. modification of the sugar.

In some embodiments, the modifications listed above can be combined to provide modified nucleosides and nucleotides that can have two, three, four, or more modifications. For example, in some embodiments, a modified nucleoside or nucleotide can have a modified sugar and a modified nucleobase. In some embodiments, every base of a gRNA is modified. In some embodiments, each of the phosphate groups of a gRNA molecule are replaced with phosphorothioate groups.

In some embodiments, a software tool can be used to optimize the choice of gRNA within a user's target sequence, e.g., to minimize total off-target activity across the genome. Off target activity may be other than cleavage. For example, for each possible gRNA choice using S. pyogenes Cas9, software tools can identify all potential off-target sequences (preceding either NAG or NGG PAMs) across the genome that contain up to a certain number (e.g., 1, 2, 3, 4, 5, 6, 7, 8, 9, or 10) of mismatched base-pairs. The cleavage efficiency at each off-target sequence can be predicted, e.g., using an experimentally-derived weighting scheme. Each possible gRNA can then be ranked according to its total predicted off-target cleavage; the top-ranked gRNAs represent those that are likely to have the greatest on-target and the least off-target cleavage. Other functions, e.g., automated reagent design for gRNA vector construction, primer design for the on-target Surveyor assay, and primer design for high-throughput detection and quantification of off-target cleavage via next-generation sequencing, can also be included in the tool.

In some embodiments, the present disclosure provides polynucleotides or nucleic acid molecules encoding a gene-regulating system described herein. Polynucleotides may be single-stranded or double-stranded and either recombinant, synthetic, or isolated. Polynucleotides include, but are not limited to: pre-messenger RNA (pre-mRNA), messenger RNA (mRNA), RNA, genomic DNA (gDNA), PCR amplified DNA, complementary DNA (cDNA), synthetic DNA, or recombinant DNA. Polynucleotides refer to a polymeric form of nucleotides of at least 5, at least 10, at least 15, at least 20, at least 25, at least 30, at least 40, at least 50, at least 100, at least 200, at least 300, at least 400, at least 500, at least 1000, at least 5000, at least 10000, or at least 15000 or more nucleotides in length, either ribonucleotides or deoxyribonucleotides or a modified form of either type of nucleotide, as well as all intermediate lengths. It will be readily understood that “intermediate lengths,” in this context, means any length between the quoted values, such as 6, 7, 8, 9, etc., 101, 102, 103, etc.; 151, 152, 153, etc.; 201, 202, 203, etc.

In particular embodiments, polynucleotides may be codon-optimized. As used herein, the term “codon-optimized” refers to substituting codons in a polynucleotide encoding a polypeptide in order to increase the expression, stability and/or activity of the polypeptide. Factors that influence codon optimization include, but are not limited to one or more of: (i) variation of codon biases between two or more organisms or genes or synthetically constructed bias tables, (ii) variation in the degree of codon bias within an organism, gene, or set of genes, (iii) systematic variation of codons including context, (iv) variation of codons according to their decoding tRNAs, (v) variation of codons according to GC %, either overall or in one position of the triplet, (vi) variation in degree of similarity to a reference sequence for example a naturally occurring sequence, (vii) variation in the codon frequency cutoff, (viii) structural properties of mRNAs transcribed from the DNA sequence, (ix) prior knowledge about the function of the DNA sequences upon which design of the codon substitution set is to be based, (x) systematic variation of codon sets for each amino acid, (xi) isolated removal of spurious translation initiation sites and/or (xii) elimination of fortuitous polyadenylation sites otherwise leading to truncated RNA transcripts.

As used herein, the terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms include polynucleotides in which one or more nucleotides have been added or deleted, or replaced with different nucleotides compared to a reference polynucleotide. In this regard, it is well understood in the art that certain alterations inclusive of mutations, additions, deletions and substitutions can be made to a reference polynucleotide whereby the altered polynucleotide retains the biological function or activity of the reference polynucleotide.

In particular embodiments, polynucleotides or variants have at least or about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a reference sequence.

Moreover, it will be appreciated by those of ordinary skill in the art that, as a result of the degeneracy of the genetic code, there are many nucleotide sequences that encode a polypeptide, or fragment of variant thereof, as described herein. Some of these polynucleotides bear minimal homology to the nucleotide sequence of any native gene. Nonetheless, polynucleotides that vary due to differences in codon usage are specifically contemplated in particular embodiments, for example polynucleotides that are optimized for human and/or primate codon selection. Further, alleles of the genes comprising the polynucleotide sequences provided herein may also be used. Alleles are endogenous genes that are altered as a result of one or more mutations, such as deletions, additions and/or substitutions of nucleotides.

The polynucleotides contemplated herein, regardless of the length of the coding sequence itself, may be combined with other DNA sequences, such as promoters and/or enhancers, untranslated regions (UTRs), signal sequences, Kozak sequences, polyadenylation signals, additional restriction enzyme sites, multiple cloning sites, internal ribosomal entry sites (IRES), recombinase recognition sites (e.g., LoxP, FRT, and Att sites), termination codons, transcriptional termination signals, and polynucleotides encoding self-cleaving polypeptides, epitope tags, as disclosed elsewhere herein or as known in the art, such that their overall length may vary considerably. It is therefore contemplated that a polynucleotide fragment of almost any length may be employed in particular embodiments, with the total length preferably being limited by the ease of preparation and use in the intended recombinant DNA protocol.

Polynucleotides can be prepared, manipulated and/or expressed using any of a variety of well-established techniques known and available in the art.

### Polynucleotides

In some embodiments, the present disclosure provides polynucleotides or nucleic acid molecules encoding a gene-regulating system described herein. Polynucleotides may be single-stranded or double-stranded and either recombinant, synthetic, or isolated. Polynucleotides include, but are not limited to: pre-messenger RNA (pre-mRNA), messenger RNA (mRNA), RNA, genomic DNA (gDNA), PCR amplified DNA, complementary DNA (cDNA), synthetic DNA, or recombinant DNA. Polynucleotides refer to a polymeric form of nucleotides of at least 5, at least 10, at least 15, at least 20, at least 25, at least 30, at least 40, at least 50, at least 100, at least 200, at least 300, at least 400, at least 500, at least 1000, at least 5000, at least 10000, or at least 15000 or more nucleotides in length, either ribonucleotides or deoxyribonucleotides or a modified form of either type of nucleotide, as well as all intermediate lengths. It will be readily understood that “intermediate lengths,” in this context, means any length between the quoted values, such as 6, 7, 8, 9, etc., 101, 102, 103, etc.; 151, 152, 153, etc.; 201, 202, 203, etc.

In particular embodiments, polynucleotides may be codon-optimized. As used herein, the term “codon-optimized” refers to substituting codons in a polynucleotide encoding a polypeptide in order to increase the expression, stability and/or activity of the polypeptide. Factors that influence codon optimization include, but are not limited to one or more of: (i) variation of codon biases between two or more organisms or genes or synthetically constructed bias tables, (ii) variation in the degree of codon bias within an organism, gene, or set of genes, (iii) systematic variation of codons including context, (iv) variation of codons according to their decoding tRNAs, (v) variation of codons according to GC %, either overall or in one position of the triplet, (vi) variation in degree of similarity to a reference sequence for example a naturally occurring sequence, (vii) variation in the codon frequency cutoff, (viii) structural properties of mRNAs transcribed from the DNA sequence, (ix) prior knowledge about the function of the DNA sequences upon which design of the codon substitution set is to be based, (x) systematic variation of codon sets for each amino acid, (xi) isolated removal of spurious translation initiation sites and/or (xii) elimination of fortuitous polyadenylation sites otherwise leading to truncated RNA transcripts.

As used herein, the terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms include polynucleotides in which one or more nucleotides have been added or deleted, or replaced with different nucleotides compared to a reference polynucleotide. In this regard, it is well understood in the art that certain alterations inclusive of mutations, additions, deletions and substitutions can be made to a reference polynucleotide whereby the altered polynucleotide retains the biological function or activity of the reference polynucleotide.

In particular embodiments, polynucleotides or variants have at least or about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a reference sequence.

Moreover, it will be appreciated by those of ordinary skill in the art that, as a result of the degeneracy of the genetic code, there are many nucleotide sequences that encode a polypeptide, or fragment of variant thereof, as described herein. Some of these polynucleotides bear minimal homology to the nucleotide sequence of any native gene. Nonetheless, polynucleotides that vary due to differences in codon usage are specifically contemplated in particular embodiments, for example polynucleotides that are optimized for human and/or primate codon selection. Further, alleles of the genes comprising the polynucleotide sequences provided herein may also be used. Alleles are endogenous genes that are altered as a result of one or more mutations, such as deletions, additions and/or substitutions of nucleotides.

The polynucleotides contemplated herein, regardless of the length of the coding sequence itself, may be combined with other DNA sequences, such as promoters and/or enhancers, untranslated regions (UTRs), signal sequences, Kozak sequences, polyadenylation signals, additional restriction enzyme sites, multiple cloning sites, internal ribosomal entry sites (IRES), recombinase recognition sites (e.g., LoxP, FRT, and Att sites), termination codons, transcriptional termination signals, and polynucleotides encoding self-cleaving polypeptides, epitope tags, as disclosed elsewhere herein or as known in the art, such that their overall length may vary considerably. It is therefore contemplated that a polynucleotide fragment of almost any length may be employed in particular embodiments, with the total length preferably being limited by the ease of preparation and use in the intended recombinant DNA protocol.

Polynucleotides can be prepared, manipulated and/or expressed using any of a variety of well-established techniques known and available in the art.

### Vectors

In order to express a gene-regulating system described herein in a cell, an expression cassette encoding the gene-regulating system can be inserted into appropriate vector. The term “nucleic acid vector” is used herein to refer to a nucleic acid molecule capable transferring or transporting another nucleic acid molecule. The transferred nucleic acid is generally linked to, e.g., inserted into, the vector nucleic acid molecule. A nucleic acid vector may include sequences that direct autonomous replication in a cell, or may include sequences sufficient to allow integration into host cell DNA.

In particular embodiments, vectors include, without limitation, plasmids, phagemids, cosmids, transposons, artificial chromosomes such as yeast artificial chromosome (YAC), bacterial artificial chromosome (BAC), or P1-derived artificial chromosome (PAC), bacteriophages such as lambda phage or M13 phage, and animal viruses. In particular embodiments, the coding sequences of the gene-regulating systems disclosed herein can be ligated into such vectors for the expression of the gene-regulating systems in mammalian cells.

In some embodiments, non-viral vectors are used to deliver one or more polynucleotides contemplated herein to an pluripotent stem cell, e.g., a T cell. In some embodiments, the recombinant vector comprising a polynucleotide encoding one or more components of a gene-regulating system described herein is a plasmid. Numerous suitable plasmid expression vectors are known to those of skill in the art, and many are commercially available. The following vectors are provided by way of example; for eukaryotic host cells: pXT1, pSG5 (Stratagene), pSVK3, pBPV, pMSG, and pSVLSV40 (Pharmacia). However, any other plasmid vector may be used so long as it is compatible with the host cell. Depending on the cell type and gene-regulating system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544).

In some embodiments, the recombinant vector comprising a polynucleotide encoding one or more components of a gene-regulating system described herein is a viral vector. Suitable viral vectors include, but are not limited to, viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., U.S. Pat. No. 7,078,387; Ali et al., Hum Gene Ther 9:81 86, 1998, Flannery et al, PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al, Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like. Examples of vectors are pClneo vectors (Promega) for expression in mammalian cells; pLenti4/V5-DEST™, pLenti6/V5-DEST™, and pLenti6.2/V5-GW/lacZ (Invitrogen) for lentivirus-mediated gene transfer and expression in mammalian cells.

In some embodiments, the vector is a non-integrating vector, including but not limited to, an episomal vector or a vector that is maintained extrachromosomally. As used herein, the term “episomal” refers to a vector that is able to replicate without integration into host's chromosomal DNA and without gradual loss from a dividing host cell also meaning that said vector replicates extrachromosomally or episomally. The vector is engineered to harbor the sequence coding for the origin of DNA replication or “ori” from a lymphotrophic herpes virus or a gamma herpesvirus, an adenovirus, SV40, a bovine papilloma virus, or a yeast, specifically a replication origin of a lymphotrophic herpes virus or a gamma herpesvirus corresponding to oriP of EBV. In a particular aspect, the lymphotrophic herpes virus may be Epstein Barr virus (EBV), Kaposi's sarcoma herpes virus (KSHV), Herpes virus saimiri (HS), or Marek's disease virus (MDV). Epstein Barr virus (EBV) and Kaposi's sarcoma herpes virus (KSHV) are also examples of a gamma herpesvirus.

In some embodiments, a polynucleotide is introduced into a target or host cell using a transposon vector system. In certain embodiments, the transposon vector system comprises a vector comprising transposable elements and a polynucleotide contemplated herein; and a transposase. In one embodiment, the transposon vector system is a single transposase vector system, see, e.g., WO 2008/027384. Exemplary transposases include, but are not limited to: piggyBac, Sleeping Beauty, Mos1, Tc1/mariner, Tol2, mini-Tol2, Tc3, MuA, Himar I, Frog Prince, and derivatives thereof. The piggyBac transposon and transposase are described, for example, in U.S. Pat. No. 6,962,810, which is incorporated herein by reference in its entirety. The Sleeping Beauty transposon and transposase are described, for example, in Izsvak et al., J. Mol. Biol. 302: 93-102 (2000), which is incorporated herein by reference in its entirety. The Tol2 transposon which was first isolated from the medaka fish Oryzias latipes and belongs to the hAT family of transposons is described in Kawakami et al. (2000). Mini-Tol2 is a variant of Tol2 and is described in Balciunas et al. (2006). The Tol2 and Mini-Tol2 transposons facilitate integration of a transgene into the genome of an organism when co-acting with the Tol2 transposase. The Frog Prince transposon and transposase are described, for example, in Miskey et al., Nucleic Acids Res. 31:6873-6881 (2003).

In some embodiments, a polynucleotide sequence encoding one or more components of a gene-regulating system described herein is operably linked to a control element, e.g., a transcriptional control element, such as a promoter. “Control elements” refer those non-translated regions of the vector (e.g., origin of replication, selection cassettes, promoters, enhancers, translation initiation signals (Shine Dalgarno sequence or Kozak sequence) introns, a polyadenylation sequence, 5′ and 3′ untranslated regions) which interact with host cellular proteins to carry out transcription and translation. Such elements may vary in their strength and specificity. The transcriptional control element may be functional in either a eukaryotic cell (e.g., a mammalian cell) or a prokaryotic cell (e.g., bacterial or archaeal cell). In some embodiments, a polynucleotide sequence encoding one or more components of a gene-regulating system described herein is operably linked to multiple control elements that allow expression of the polynucleotide in both prokaryotic and eukaryotic cells.

Depending on the cell type and gene-regulating system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544). An RNA polymerase initiates and transcribes polynucleotides operably linked to the promoter. In particular embodiments, promoters operative in mammalian cells comprise an AT-rich region located approximately 25 to 30 bases upstream from the site where transcription is initiated and/or another sequence found 70 to 80 bases upstream from the start of transcription, a CNCAAT region where N may be any nucleotide. The term “enhancer” refers to a segment of DNA which contains sequences capable of providing enhanced transcription and in some instances can function independent of their orientation relative to another control sequence. An enhancer can function cooperatively or additively with promoters and/or other enhancer elements.

In some embodiments, polynucleotides encoding one or more components of a gene-regulating system described herein are operably linked to a promoter. The term “operably linked”, refers to a juxtaposition wherein the components described are in a relationship permitting them to function in their intended manner. In one embodiment, the term refers to a functional linkage between a nucleic acid expression control sequence (such as a promoter, and/or enhancer) and a second polynucleotide sequence, e.g., a polynucleotide encoding one or more components of a gene-regulating system, wherein the expression control sequence directs transcription of the nucleic acid corresponding to the second sequence.

Non-limiting examples of suitable eukaryotic promoters (promoters functional in a eukaryotic cell) include those from cytomegalovirus (CMV) immediate early, herpes simplex virus (HSV) thymidine kinase, a viral simian virus 40 (SV40) (e.g., early and late SV40), a spleen focus forming virus (SFFV) promoter, long terminal repeats (LTRs) from retrovirus (e.g., a Moloney murine leukemia virus (MoMLV) LTR promoter or a Rous sarcoma virus (RSV) LTR), a herpes simplex virus (HSV) (thymidine kinase) promoter, H5, P7.5, and P11 promoters from vaccinia virus, an elongation factor 1-alpha (EF1α) promoter, early growth response 1 (EGR1) promoter, a ferritin H (FerH) promoter, a ferritin L (FerL) promoter, a Glyceraldehyde 3-phosphate dehydrogenase (GAPDH) promoter, a eukaryotic translation initiation factor 4A1 (EIF4A1) promoter, a heat shock 70 kDa protein 5 (HSPA5) promoter, a heat shock protein 90 kDa beta, member 1 (HSP90B1) promoter, a heat shock protein 70 kDa (HSP70) promoter, a 0-kinesin (0-KIN) promoter, the human ROSA 26 locus (Irions et al., Nature Biotechnology 25, 1477-1482 (2007)), a Ubiquitin C (UBC) promoter, a phosphoglycerate kinase-1 (PGK) promoter, a cytomegalovirus enhancer/chicken 3-actin (CAG) promoter, a 3-actin promoter and a myeloproliferative sarcoma virus enhancer, negative control region deleted, dl587rev primer-binding site substituted (MND) promoter, and mouse metallothionein-1. Selection of the appropriate vector and promoter is well within the level of ordinary skill in the art. The expression vector may also contain a ribosome binding site for translation initiation and a transcription terminator. The expression vector may also include appropriate sequences for amplifying expression. The expression vector may also include nucleotide sequences encoding protein tags (e.g., 6×His tag, hemagglutinin tag, green fluorescent protein, etc.) that are fused to the site-directed modifying polypeptide, thus resulting in a chimeric polypeptide.

In some embodiments, a polynucleotide sequence encoding one or more components of a gene-regulating system described herein is operably linked to a constitutive promoter. In such embodiments, the polynucleotides encoding one or more components of a gene-regulating system described herein are constitutively and/or ubiquitously expressed in a cell.

In some embodiments, a polynucleotide sequence encoding one or more components of a gene-regulating system described herein is operably linked to an inducible promoter. In such embodiments, polynucleotides encoding one or more components of a gene-regulating system described herein are conditionally expressed. As used herein, “conditional expression” may refer to any type of conditional expression including, but not limited to, inducible expression; repressible expression; expression in cells or tissues having a particular physiological, biological, or disease state (e.g., cell type or tissue specific expression) etc. Illustrative examples of inducible promoters/systems include, but are not limited to, steroid-inducible promoters such as promoters for genes encoding glucocorticoid or estrogen receptors (inducible by treatment with the corresponding hormone), metallothionine promoter (inducible by treatment with various heavy metals), MX-1 promoter (inducible by interferon), the “GeneSwitch” mifepristone-regulatable system (Sirin et al., 2003, Gene, 323:67), the cumate inducible gene switch (WO 2002/088346), tetracycline-dependent regulatory systems, etc.

In some embodiments, the vectors described herein further comprise a transcription termination signal. Elements directing the efficient termination and polyadenylation of the heterologous nucleic acid transcripts increases heterologous gene expression. Transcription termination signals are generally found downstream of the polyadenylation signal. In particular embodiments, vectors comprise a polyadenylation sequence 3′ of a polynucleotide encoding a polypeptide to be expressed. The term “polyA site” or “polyA sequence” as used herein denotes a DNA sequence which directs both the termination and polyadenylation of the nascent RNA transcript by RNA polymerase II. Polyadenylation sequences can promote mRNA stability by addition of a polyA tail to the 3′ end of the coding sequence and thus, contribute to increased translational efficiency. Cleavage and polyadenylation is directed by a poly(A) sequence in the RNA. The core poly(A) sequence for mammalian pre-mRNAs has two recognition elements flanking a cleavage-polyadenylation site. Typically, an almost invariant AAUAAA hexamer lies 20-50 nucleotides upstream of a more variable element rich in U or GU residues. Cleavage of the nascent transcript occurs between these two elements and is coupled to the addition of up to 250 adenosines to the 5′ cleavage product. In particular embodiments, the core poly(A) sequence is an ideal polyA sequence (e.g., AATAAA, ATTAAA, AGTAAA). In particular embodiments, the poly(A) sequence is an SV40 polyA sequence, a bovine growth hormone polyA sequence (BGHpA), a rabbit β-globin polyA sequence (rpgpA), variants thereof, or another suitable heterologous or endogenous polyA sequence known in the art.

In some embodiments, a vector may also comprise a sequence encoding a signal peptide (e.g., for nuclear localization, nucleolar localization, mitochondrial localization), fused to the polynucleotide encoding the one or more components of the system. For example, a vector may comprise a nuclear localization sequence (e.g., from SV40) fused to the polynucleotide encoding the one or more components of the system.

Methods of introducing polynucleotides and recombinant vectors into a host cell are known in the art, and any known method can be used to introduce components of a gene-regulating system into a cell. Suitable methods include e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et al., Adv Drug Deliv Rev. 2012 Sep. 13. pii: S0169-409X(12)00283-9), microfluidics delivery methods (See e.g., International PCT Publication No. WO 2013/059343), and the like. In some embodiments, delivery via electroporation comprises mixing the cells with the components of a gene-regulating system in a cartridge, chamber, or cuvette and applying one or more electrical impulses of defined duration and amplitude. In some embodiments, cells are mixed with components of a gene-regulating system in a vessel connected to a device (e.g., a pump) which feeds the mixture into a cartridge, chamber, or cuvette wherein one or more electrical impulses of defined duration and amplitude are applied, after which the cells are delivered to a second vessel. Illustrative examples of polynucleotide delivery systems suitable for use in particular embodiments contemplated in particular embodiments include, but are not limited to, those provided by Amaxa Biosystems, Maxcyte, Inc., BTX Molecular Delivery Systems, Neon™ Transfection Systems, and Copernicus Therapeutics Inc. Lipofection reagents are sold commercially (e.g., Transfectam™ and Lipofectin™). Cationic and neutral lipids that are suitable for efficient lipofection of polynucleotides have been described in the literature. See e.g., Liu et al. (2003) Gene Therapy. 10:180-187; and Balazs et al. (2011) Journal of Drug Delivery. 2011:1-12.

In some embodiments, vectors comprising polynucleotides encoding one or more components of a gene-regulating system described herein are introduced to cells by viral delivery methods, e.g., by viral transduction. In some embodiments, vectors comprising polynucleotides encoding one or more components of a gene-regulating system described herein are introduced to cells by non-viral delivery methods. Illustrative methods of non-viral delivery of polynucleotides contemplated in particular embodiments include, but are not limited to: electroporation, sonoporation, lipofection, microinjection, biolistics, virosomes, liposomes, immunoliposomes, nanoparticles, polycation or lipid:nucleic acid conjugates, naked DNA, artificial virions, DEAE-dextran-mediated transfer, gene gun, and heat-shock.

In some embodiments, one or more components of a gene-regulating system, or polynucleotide sequence encoding one or more components of a gene-regulating system described herein are introduced to a cell in a non-viral delivery vehicle, such as a transposon, a nanoparticle (e.g., a lipid nanoparticle), a liposome, an exosome, an attenuated bacterium, or a virus-like particle. In some embodiments, the vehicle is an attenuated bacterium (e.g., naturally or artificially engineered to be invasive but attenuated to prevent pathogenesis including Listeria monocytogenes, certain Salmonella strains, Bifidobacterium longum, and modified Escherichia coli), bacteria having nutritional and tissue-specific tropism to target specific cells, and bacteria having modified surface proteins to alter target cell specificity. In some embodiments, the vehicle is a genetically modified bacteriophage (e.g., engineered phages having large packaging capacity, less immunogenicity, containing mammalian plasmid maintenance sequences and having incorporated targeting ligands). In some embodiments, the vehicle is a mammalian virus-like particle. For example, modified viral particles can be generated (e.g., by purification of the “empty” particles followed by ex vivo assembly of the virus with the desired cargo). The vehicle can also be engineered to incorporate targeting ligands to alter target tissue specificity. In some embodiments, the vehicle is a biological liposome. For example, the biological liposome is a phospholipid-based particle derived from human cells (e.g., erythrocyte ghosts, which are red blood cells broken down into spherical structures derived from the subject and wherein tissue targeting can be achieved by attachment of various tissue or cell-specific ligands), secretory exosomes, or subject derived membrane-bound nanovesicles (30-100 nm) of endocytic origin (e.g., can be produced from various cell types and can therefore be taken up by cells without the need for targeting ligands).

### AAV Viral Vectors

In some embodiments, the vector is a viral vector. A number of viral based systems have been developed for gene transfer into mammalian cells. Examples of viral vectors include, but are not limited to, adenoviral vectors, adeno-associated virus vectors, lentiviral vector, retroviral vectors, vaccinia vector, herpes simplex viral vector, and derivatives thereof. Viral vector technology is well known in the art and is described, for example, in Sambrook et al. (2001, Molecular Cloning: A Laboratory Manual, Cold Spring Harbor Laboratory, New York), and in other virology and molecular biology manuals. In some embodiments, the viral vector is a recombinant adeno-associated virus (rAAV) vector.

The rAAV vector can be derived from any AAV serotypes or variants thereof. In some embodiments, the rAAV vector is derived from AAV1 or a variant thereof. In some embodiments, the rAAV vector is derived from AAV2 or a variant thereof. In some embodiments, the rAAV vector is derived from AAV2i8 or a variant thereof. In some embodiments, the rAAV vector is derived from AAV3 or a variant thereof. In some embodiments, the rAAV vector is derived from AAV3-B or a variant thereof. In some embodiments, the rAAV vector is derived from AAV4 or a variant thereof. In some embodiments, the rAAV vector is derived from AAV5 or a variant thereof. In some embodiments, the rAAV vector is derived from AAV6 or a variant thereof. In some embodiments, the rAAV vector is derived from AAV7 or a variant thereof. In some embodiments, the rAAV vector is derived from AAV8 or a variant thereof. In some embodiments, the rAAV vector is derived from AAVrh8 or a variant thereof. In some embodiments, the rAAV vector is derived from AAVrh8R or a variant thereof. In some embodiments, the rAAV vector is derived from AAV9 or a variant thereof. In some embodiments, the rAAV vector is derived from AAV10 or a variant thereof. In some embodiments, the rAAV vector is derived from AAVrh10 or a variant thereof. In some embodiments, the rAAV vector is derived from AAV11 or a variant thereof. In some embodiments, the rAAV vector is derived from AAV12 or a variant thereof.

In some embodiments, the AAV vector is an AVV8 vector, an AAV1 vector, an AAV6.2 vector, an AAVrh74 vector, an AAV9 vector, or an AAVPHP.s. An AAV vector can be implemented in dual AAV-mediated delivery. In some embodiments, the AAV mediated delivery comprises TRPV1, sgRNA and SaCas9. In some embodiments, the AAV mediated delivery comprises SCN9A, sgRNA and SaCas9. In some embodiments, the AAV mediated delivery comprises KCNQ2. In some embodiments, the dual AAV-mediated delivery comprises a nucleic acid sequence comprising at least about 85%, at least about 90%, at least about 95%, or at least about 100% identity to SEQ ID NOs: 5-7. In some embodiments, the dual AAV-mediated delivery comprises a nucleic acid sequence comprising at least about 85%, at least about 90%, at least about 95%, or at least about 100% identity to SEQ ID NOs: 5, 8 and 9. In some embodiments, the dual AAV-mediated delivery comprises a nucleic acid sequence comprising at least about 85%, at least about 90%, at least about 95%, or at least about 100% identity to SEQ ID NOs: 11, 12, or 13. In some embodiments, the dual AAV-mediated delivery comprises a nucleic acid sequence comprising at least about 85%, at least about 90%, at least about 95%, or at least about 100% identity to SEQ ID NOs: 14, 15, or 16.

### Methods of Producing Modified Pluripotent Stem Cells

In some embodiments, the present disclosure provides methods for producing modified stem cells. In some embodiments, the present disclosure provides methods for producing modified pluripotent stem cells. In some embodiments, the methods comprise introducing a gene-regulating system into a population of pluripotent stem cells wherein the gene-regulating system is capable of reducing expression and/or function of one or more endogenous target genes.

The components of the gene-regulating systems described herein, e.g., a nucleic acid-, protein-, or nucleic acid/protein-based system can be introduced into target cells in a variety of forms using a variety of delivery methods and formulations. In some embodiments, a polynucleotide encoding one or more components of the system is delivered by a recombinant vector (e.g., a viral vector or plasmid, described supra). In some embodiments, where the system comprises more than a single component, a vector may comprise a plurality of polynucleotides, each encoding a component of the system. In some embodiments, where the system comprises more than a single component, a plurality of vectors may be used, wherein each vector comprises a polynucleotide encoding a particular component of the system. In some embodiments, the introduction of the gene-regulating system to the cell occurs in vitro. In some embodiments, the introduction of the gene-regulating system to the cell occurs in vivo. In some embodiments, the introduction of the gene-regulating system to the cell occurs ex vivo.

In particular embodiments, the introduction of the gene-regulating system to the cell occurs in vitro or ex vivo. In some embodiments, the pluripotent stem cells are modified in vitro or ex vivo without further manipulation in culture. For example, in some embodiments, the methods of producing a modified pluripotent stem cell described herein comprise introduction of a gene-regulating system in vitro or ex vivo without additional activation and/or expansion steps. In some embodiments, the pluripotent stem cells are modified and are further manipulated in vitro or ex vivo. For example, in some embodiments, the pluripotent stem cells are activated and/or expanded in vitro or ex vivo prior to introduction of a gene-regulating system. In some embodiments, a gene-regulating system is introduced to the pluripotent stem cells and are then activated and/or expanded in vitro or ex vivo. In some embodiments, successfully modified cells can be sorted and/or isolated (e.g., by flow cytometry) from unsuccessfully modified cells to produce a purified population of modified pluripotent stem cells. These successfully modified cells can then be further propagated to increase the number of the modified cells and/or cryopreserved for future use.

In some embodiments, the present disclosure provides methods for producing modified pluripotent stem cells comprising obtaining a population of pluripotent stem cells. The population of pluripotent stem cells may be cultured in vitro under various culture conditions necessary to support growth, for example, at an appropriate temperature (e.g., 37° C.) and atmosphere (e.g., air plus 5% CO₂) and in an appropriate culture medium. Culture medium may be liquid or semi-solid, e.g. containing agar, methylcellulose, etc. Illustrative examples of cell culture media include Minimal Essential Media (MEM), Iscove's modified DMEM, RPMI 1640Clicks, AIM-V, F-12, X-Vivo 15, X-Vivo 20, and Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of the pluripotent stem cells.

Culture media may be supplemented with one or more factors necessary for proliferation and viability including, but not limited to, growth factors such as serum (e.g., fetal bovine or human serum at about 5%-10%), interleukin-2 (IL-2), insulin, IFN-7, IL-4, IL-7, IL-21, GM-CSF, IL-10, IL-12, IL-15, TGFβ, and TNF-α. Illustrative examples of other additives for T cell expansion include, but are not limited to, surfactant, piasmanate, pH buffers such as HEPES, and reducing agents such as N-acetyl-cysteine and 2-mercaptoethanol, or any other additives suitable for the growth of cells known to the skilled artisan such as L-glutamine, a thiol, particularly 2-mercaptoethanol, and/or antibiotics, e.g. penicillin and streptomycin. Typically, antibiotics are included only in experimental cultures, not in cultures of cells that are to be infused into a subject.

In some embodiments, the population of pluripotent stem cells is obtained from a sample derived from a subject. In some embodiments, a population of pluripotent stem cells is obtained is obtained from a first subject and the population of modified pluripotent stem cells produced by the methods described herein is administered to a second, different subject. In some embodiments, a population of pluripotent stem cells is obtained from a subject and the population of modified pluripotent stem cells produced by the methods described herein is administered to the same subject. In some embodiments, the sample is a tissue sample, a fluid sample, a cell sample, a protein sample, or a DNA or RNA sample. In some embodiments, a tissue sample may be derived from any tissue type including, but not limited to skin, hair (including roots), bone marrow, bone, muscle, salivary gland, esophagus, stomach, small intestine (e.g., tissue from the duodenum, jejunum, or ileum), large intestine, liver, gallbladder, pancreas, lung, kidney, bladder, uterus, ovary, vagina, placenta, testes, thyroid, adrenal gland, cardiac tissue, thymus, spleen, lymph node, spinal cord, brain, eye, ear, tongue, cartilage, white adipose tissue, or brown adipose tissue. In some embodiments, a tissue sample may be derived from a cancerous, pre-cancerous, or non-cancerous tumor. In some embodiments, a fluid sample comprises buccal swabs, blood, plasma, oral mucous, vaginal mucous, peripheral blood, cord blood, saliva, semen, urine, ascites fluid, pleural fluid, spinal fluid, pulmonary lavage, tears, sweat, semen, seminal fluid, seminal plasma, prostatic fluid, pre-ejaculatory fluid (Cowper's fluid), excreta, cerebrospinal fluid, lymph, cell culture media comprising one or more populations of cells, buffered solutions comprising one or more populations of cells, and the like.

In some embodiments, the sample is processed to enrich or isolate a population of pluripotent stem cells from the remainder of the sample. In certain embodiments, the sample is a peripheral blood sample which is then subject to leukapheresis to separate the red blood cells and platelets and to isolate lymphocytes. In some embodiments, the sample is a leukopak from which pluripotent stem cells can be isolated or enriched. In some embodiments, the sample is a tumor sample that is further processed to isolate lymphocytes present in the tumor (i.e., by fragmentation and enzymatic digestion of the tumor to obtain a cell suspension of tumor infiltrating lymphocytes).

In some embodiments, a method for manufacturing modified stems cells contemplated herein comprises activating a population of cells comprising stem cells. In particular embodiments, the stems cells comprise totipotent, pluripotent, multipotent, oligopotent, and unipotent.

In some embodiments, a method for manufacturing modified pluripotent stems cells contemplated herein comprises activating a population of cells comprising pluripotent stem cells. In particular embodiments, the pluripotent stems cells comprise embryonic stem cells (ESCs), induced pluripotent stem cells (iPSCs), epiblast stem cells (EpiSCs), embryonic germ cells (EGCs), and nuclear transfer embryonic stem cells (ntESCs).

Provided herein are methods of producing an isolated population of cells comprising human pluripotent stem cell-derived nociceptive neurons (hPSC-NNs) expressing CD200^{low} with altered expression of KCNQ2, TRPV1, or SCN9A. In some embodiments, the cells are contacted with a composition for base editing KCNQ2 mutations, wherein the composition comprises a system comprising a base editor and a gRNA targeting KCNQ2.

In some embodiments, the base editor comprises ABE8e, ABE8e(V106W), ABE9, ABE20, ABE7.10, SaKKHABE8e, SauriCas9-ABE8e, CjCas9-ABE8e, Nme2Cas9-ABE8e, or SaCas9-ABE8e. In some embodiments, the cells are contacted with a composition for silencing expression of TRPV1 and SCN9A, wherein the composition comprises a system comprising a Cas9 endonuclease and a gRNA targeting TRPV1 and SCN9A.

### Producing Modified Pluripotent Stems Cells Using CRISPR/Cas9 Systems

In some embodiments, a method of producing a modified stem cell involves contacting a target DNA sequence with a complex comprising a gRNA and a Cas polypeptide. In some embodiments, a method of producing a modified stem cell involves contacting a target DNA sequence with a complex comprising a gRNA and a Cas polypeptide. As discussed above, a gRNA and Cas polypeptide form a complex, wherein the DNA-binding domain of the gRNA targets the complex to a target DNA sequence and wherein the Cas protein (or heterologous protein fused to an enzymatically inactive Cas protein) modifies target DNA sequence. In some embodiments, this complex is formed intracellularly after introduction of the gRNA and Cas protein (or polynucleotides encoding the gRNA and Cas proteins) to a cell. In some embodiments, the nucleic acid encoding the Cas protein is a DNA nucleic acid and is introduced to the cell by transduction. In some embodiments, the Cas9 and gRNA components of a CRISPR/Cas gene editing system are encoded by a single polynucleotide molecule. In some embodiments, the polynucleotide encoding the Cas protein and gRNA component are comprised in a viral vector and introduced to the cell by viral transduction. In some embodiments, the Cas9 and gRNA components of a CRISPR/Cas gene editing system are encoded by different polynucleotide molecules. In some embodiments, the polynucleotide encoding the Cas protein is comprised in a first viral vector and the polynucleotide encoding the gRNA is comprised in a second viral vector. In some aspects of this embodiment, the first viral vector is introduced to a cell prior to the second viral vector. In some aspects of this embodiment, the second viral vector is introduced to a cell prior to the first viral vector. In such embodiments, integration of the vectors results in sustained expression of the Cas9 and gRNA components. However, sustained expression of Cas9 may lead to increased off-target mutations and cutting in some cell types. Therefore, in some embodiments, an mRNA nucleic acid sequence encoding the Cas protein may be introduced to the population of cells by transfection. In such embodiments, the expression of Cas9 will decrease over time, and may reduce the number of off target mutations or cutting sites. In some embodiments, the gRNA and Cas protein are introduced separately by electroporation.

In some embodiments, this complex is formed in a cell-free system by mixing the gRNA molecules and Cas proteins together and incubating for a period of time sufficient to allow complex formation. This pre-formed complex, comprising the gRNA and Cas protein and referred to herein as a CRISPR-ribonucleoprotein (CRISPR-RNP) can then be introduced to a cell in order to modify a target DNA sequence. In some embodiments, the CRISPR-RNP is introduced to the cell by electroporation.

In any of the above described embodiments for producing a modified pluripotent stem cell using the CRISPR/Cas system, the system may comprise one or more gRNAs targeting a single endogenous target gene, for example to produce a single-edited modified pluripotent stem cell. Alternatively, in any of the above described embodiments for producing a modified pluripotent stem cell using the CRISPR/Cas system, the system may comprise two or more gRNAs targeting two or more endogenous target genes, for example to produce a dual-edited modified pluripotent stem cell.

Provided herein are methods of editing at least one target gene comprising the compositions described herein. Provided herein are also methods of producing an isolated population of cells, wherein the population of cells, the method comprising contacting the cells with a composition for base editing target gene mutations, wherein the composition comprises a system comprising a base editor and a gRNA targeting at least one gene. In some embodiments, the least one target gene comprises SCN9A, TRPV1, MRGPRX1, or KCNQ2. In some embodiments, the gRNA comprises a sequence comprising at least 80%, at least 85%, at least 90%, at least 95%, at least 99% or 100% identity to any one of SEQ ID NOs: 82-97. In some embodiments, the gRNA comprises a sequence selected from any one of SEQ ID NOs: 82-97. In some embodiments, the gRNA comprises a spacer sequence comprising at least 80%, at least 85%, at least 90%, at least 95%, at least 99% or 100% identity to any one of SEQ ID NOs: 17, 22, 27, 32, 35, 38, 41, 44, 47, 50, 53, 56, or 59-81. In some embodiments, the gRNA comprises a spacer sequence selected from any one of SEQ ID NOs: 17, 22, 27, 32, 35, 38, 41, 44, 47, 50, 53, 56, or 59-81.

In some embodiments, the base editor comprises ABE8e, ABE8e(V106W), ABE9, ABE20, ABE7.10, SaKKHABE8e, SauriCas9-ABE8e, CjCas9-ABE8e, Nme2Cas9-ABE8e, or SaCas9-ABE8e. In some embodiments, the base editor comprises Cas9 endonuclease. In some embodiments, the Cas9 endonuclease comprises Type II Cas9.

Provided herein are also methods of editing KCNQ2 mutations comprising the compositions described herein. In some embodiments, KCNQ2 function is restored. In some embodiments, KCNQ2 function is restored. In some embodiments, the KCNQ2 mutations comprises a p.T730A or c.2188A>G mutation.

Provided herein are methods of editing TRPV1 or SCN9A comprising the compositions described herein. In some embodiments, TRPV1 or SCN9A expression is reduced or eliminated.

Provided herein are also methods of producing an isolated population of cells comprising human pluripotent stem cell-derived nociceptive sensory neurons (hPSC-NSNs) expressing CD200^{high} with altered expression of KCNQ2, TRPV1, or SCN9A. In some embodiments, the cells are contacted with a composition for base editing KCNQ2 mutations, wherein the composition comprises a system comprising a base editor and a gRNA targeting KCNQ2. In some embodiments, the gRNA comprises a sequence comprising at least 80%, at least 85%, at least 90%, at least 95%, at least 99% or 100% identity to any one of SEQ ID NO: 83. In some embodiments, the gRNA comprises a sequence of SEQ ID NO: 83. In some embodiments, the gRNA comprises a spacer sequence comprising at least 80%, at least 85%, at least 90%, at least 95%, at least 99% or 100% identity to any one of SEQ ID NOs: 32, 35, 38, 41, 44, 47, 50, 53, 56, or 59-81. In some embodiments, the gRNA comprises a spacer sequence selected from any one of SEQ ID NOs: 32, 35, 38, 41, 44, 47, 50, 53, 56, or 59-81. In some embodiments, the base editor comprises ABE8e, ABE8e(V106W), ABE9, ABE20, ABE7.10, SaKKHABE8e, SauriCas9-ABE8e, CjCas9-ABE8e, Nme2Cas9-ABE8e, or SaCas9-ABE8e. In some embodiments, the cells are contacted with a composition for silencing expression of TRPV1 and SCN9A, wherein the composition comprises a system comprising a Cas9 endonuclease and a gRNA targeting TRPV1 and SCN9A. In some embodiments, the gRNA comprises a sequence comprising at least 80%, at least 85%, at least 90%, at least 95%, at least 99% or 100% identity to any one of SEQ ID NO: 86, 89-92, or 97. In some embodiments, the gRNA comprises a sequence of SEQ ID NO: 86, 89-92, or 97. In some embodiments, the gRNA comprises a spacer sequence comprising at least 80%, at least 85%, at least 90%, at least 95%, at least 99% or 100% identity to any one of SEQ ID NOs: 22 or 27. In some embodiments, the gRNA comprises a spacer sequence selected from any one of SEQ ID NOs: 22 or 27. In some embodiments, the Cas9 endonuclease comprises Type II Cas9.

### Compositions and Kits

The term “composition” as used herein can refer to a formulation of a gene-regulating system or a modified pluripotent stem cell described herein that is capable of being administered or delivered to a subject or cell. Typically, formulations include all physiologically acceptable compositions including derivatives and/or prodrugs, solvates, stereoisomers, racemates, or tautomers thereof with any physiologically acceptable carriers, diluents, and/or excipients. A “therapeutic composition” or “pharmaceutical composition” (used interchangeably herein) is a composition of a gene-regulating system or a modified pluripotent stem cell capable of being administered to a subject for the treatment of a particular disease or disorder or contacted with a cell for modification of one or more endogenous target genes.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, and/or emulsifier which has been approved by the United States Food and Drug Administration as being acceptable for use in humans and/or domestic animals. Exemplary pharmaceutically acceptable carriers include, but are not limited to, to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations. Except insofar as any conventional media and/or agent is incompatible with the agents of the present disclosure, its use in therapeutic compositions is contemplated. Supplementary active ingredients also can be incorporated into the compositions.

“Pharmaceutically acceptable salt” includes both acid and base addition salts. Pharmaceutically-acceptable salts include the acid addition salts (formed with the free amino groups of the protein) and which are formed with inorganic acids such as, for example, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, ptoluenesulfonic acid, trifluoroacetic acid, undecylenic acid, and the like. Salts formed with the free carboxyl groups can also be derived from inorganic bases such as, for example, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

In some embodiments, the present disclosure provides kits for carrying out a method described herein. In some embodiments, a kit can include: one or more nucleic acid molecules capable of reducing the expression or modifying the function of a gene product encoded by one or more endogenous target genes; one or more polynucleotides encoding a nucleic acid molecule that is capable of reducing the expression or modifying the function of a gene product encoded by one or more endogenous target genes; one or more proteins capable of reducing the expression or modifying the function of a gene product encoded by one or more endogenous target genes; one or more polynucleotides encoding a modifying protein that is capable of reducing the expression or modifying the function of a gene product encoded by one or more endogenous target genes; one or more gRNAs capable of binding to a target DNA sequence in an endogenous gene; one or more polynucleotides encoding one or more gRNAs capable of binding to a target DNA sequence in an endogenous gene; one or more site-directed modifying polypeptides capable of interacting with a gRNA and modifying a target DNA sequence in an endogenous gene; one or more polynucleotides encoding a site-directed modifying polypeptide capable of interacting with a gRNA and modifying a target DNA sequence in an endogenous gene; one or more guide DNAs (gDNAs) capable of binding to a target DNA sequence in an endogenous gene; one or more polynucleotides encoding one or more gDNAs capable of binding to a target DNA sequence in an endogenous gene; one or more site-directed modifying polypeptides capable of interacting with a gDNA and modifying a target DNA sequence in an endogenous gene; one or more polynucleotides encoding a site-directed modifying polypeptide capable of interacting with a gDNA and modifying a target DNA sequence in an endogenous gene; one or more gRNAs capable of binding to a target mRNA sequence encoded by an endogenous gene; one or more polynucleotides encoding one or more gRNAs capable of binding to a target mRNA sequence encoded by an endogenous gene; one or more site-directed modifying polypeptides capable of interacting with a gRNA and modifying a target mRNA sequence encoded by an endogenous gene; one or more polynucleotides encoding a site-directed modifying polypeptide capable of interacting with a gRNA and modifying a target mRNA sequence encoded by an endogenous gene; a modified pluripotent stem cell described herein; or any combination of the above.

In some embodiments, the kits described herein further comprise a population of pluripotent stem cells. In some embodiments, the kits described herein further comprise a population of human pluripotent stem cells. In some embodiments, the kits described herein further comprise a population of human pluripotent stem cells. In some embodiments, the kits described herein further comprise a population of human pluripotent stem cells sensory neurons. In some embodiments, the kits described herein comprise human pluripotent stem cell-derived nociceptive neurons (PSC-NNs). In some embodiments, the kits described herein comprise PSC-NNs expressing CD200^{high}. In some embodiments, the kits described herein comprise hPSC-NNs expressing CD200^{high}.

In addition to above-mentioned components, in some embodiments a kit further comprises instructions for using the components of the kit to practice the methods of the present disclosure. The instructions for practicing the methods are generally recorded on a suitable recording medium. For example, the instructions may be printed on a substrate, such as paper or plastic, etc. As such, the instructions may be present in the kits as a package insert or in the labeling of the container of the kit or components thereof (i.e., associated with the packaging or sub-packaging). In other embodiments, the instructions are present as an electronic storage data file present on a suitable computer readable storage medium, e.g. CD-ROM, diskette, flash drive, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

In some embodiments, the compositions described herein comprises an isolated population of cells comprising human pluripotent stem cell-derived nociceptive neurons (hPSC-NNs) expressing CD200^{high}. In some embodiments, at least 40%, at least 45%, at least 50%, at least 55%, at least 60%, at least 65%, at least 70%, at least 75%, at least 80%, at least 85%, at least 90%, at least 95%, at least 99% or 100% of the cells are CD200^{high} In some embodiments, the cells further express TRPV1, SCN9A, or MRGPRX1.

In some embodiments, the compositions described herein are for base editing KCNQ2 mutations. In some embodiments, the composition comprises a system comprising a base editor and a gRNA targeting KCNQ2. In some embodiments, the composition comprises a system comprising a base editor and a gRNA targeting SEQ ID NO: 10. In some embodiments, the composition is for silencing expression of TRPV1, SCN9A, and MRGPRX1. In some embodiments, the composition comprises a system comprising a Cas9 endonuclease and a gRNA targeting TRPV1 and SCN9A. In some embodiments, the composition comprises a system comprising a Cas9 endonuclease and a gRNA targeting SEQ ID NO: 1, 3 or 4.

Provided herein are compositions for base editing gene mutations, wherein the composition comprises a base editor and a guide RNA (gRNA) targeting at least one gene, wherein the at least one target gene comprises SCN9A, TRPV1, MRGPRX1, or KCNQ2. In some embodiments, the gRNA comprises a sequence comprising at least 80%, at least 85%, at least 90%, at least 95%, at least 99% or 100% identity to any one of SEQ ID NOs: 82-97. In some embodiments, the gRNA comprises a sequence selected from any one of SEQ ID NOs: 82-97. In some embodiments, the gRNA comprises a spacer sequence comprising at least 80%, at least 85%, at least 90%, at least 95%, at least 99% or 100% identity to any one of SEQ ID NOs: 17, 22, 27, 32, 35, 41, 44, 47, 50, 53, 56, or 59-81. In some embodiments, the gRNA comprises a spacer sequence selected from any one of SEQ ID NOs: 17, 22, 27, 32, 35, 41, 44, 47, 50, 53, 56, or 59-81.

In some embodiments, the base editor comprises ABE8e, ABE8e(V106W), ABE9, ABE20, ABE7.10, SaKKHABE8e, SauriCas9-ABE8e, CjCas9-ABE8e, Nme2Cas9-ABE8e, or SaCas9-ABE8e. In some embodiments, base editor comprises Cas9 endonuclease. In some embodiments, the Cas9 endonuclease comprises Type II Cas9.

Provided herein is also a composition for base editing KCNQ2 mutations, wherein the composition comprises a system comprising a base editor and a gRNA targeting KCNQ2. In some embodiments, the gRNA comprises a sequence comprising at least 80%, at least 85%, at least 90%, at least 95%, at least 99% or 100% identity to of SEQ ID NO: 83. In some embodiments, the gRNA comprises a sequence of SEQ ID NO: 83. In some embodiments, the gRNA comprises a spacer sequence comprising at least 80%, at least 85%, at least 90%, at least 95%, at least 99% or 100% identity to any one of SEQ ID NOs: 32, 35, 38, 41, 44, 47, 50, 53, 56, or 59-81. In some embodiments, the gRNA comprises a spacer sequence selected from any one of SEQ ID NOs: 32, 35, 38, 41, 44, 47, 50, 53, 56, or 59-81. In some embodiments, the base editor comprises ABE8e, ABE8e(V106W), ABE9, ABE20, ABE7.10, SaKKHABE8e, SauriCas9-ABE8e, CjCas9-ABE8e, Nme2Cas9-ABE8e, or SaCas9-ABE8e.

Provided herein is a composition for silencing expression of TRPV1 and SCN9A, wherein the composition comprises a system comprising a Cas9 endonuclease and a gRNA targeting TRPV1 and SCN9A. In some embodiments, the gRNA comprises a sequence at least 80%, at least 85%, at least 90%, at least 95%, at least 99% or 100% identity to SEQ ID NO: 86, 89-92, or 97. In some embodiments, the gRNA comprises a sequence of SEQ ID NO: 86, 89-92, or 97. In some embodiments, the gRNA comprises a spacer sequence at least 80%, at least 85%, at least 90%, at least 95%, at least 99% or 100% identity to any one of SEQ ID NOs: 22 or 27. In some embodiments, the gRNA comprises a spacer sequence selected from any one of SEQ ID NOs: 22 or 27. In some embodiments, the Cas9 endonuclease comprises Type II Cas9.

Provided herein are also pharmaceutical compositions or cell stocks comprising the various compositions of the disclosure.

### Therapeutic Methods

Provided herein are also therapeutic methods comprising the various pluripotent stem cells of the disclosure. In some embodiments, the pluripotent stem cells are unmodified. In some embodiments, pluripotent stem cells highly express CD200. In some embodiments, the pluripotent stem cells are modified pluripotent stem cells. The modified pluripotent stem cells and gene-regulating systems described herein may be used in a variety of therapeutic applications. For example, in some embodiments the modified pluripotent stem cells and/or gene-regulating systems described herein may be administered to a subject for purposes such as gene therapy, e.g. to treat a disease, pain, itching, or for biological research.

Provided herein are also methods of treating pain in a subject thereof, wherein the method comprises administering an isolated population of cells. In some embodiments, the isolated population of cells comprises pluripotent stem cells. In some embodiments, the pluripotent stem cells are human pluripotent stem cells. In some embodiments, the pluripotent stem cells highly express CD200. In some embodiments, the method of treating pain in a subject thereof comprises administering an isolated population of cells to a joint of the subject. In some embodiments, the joint of the subject is a knee joint. In some embodiments, the method of treating pain in a subject thereof comprises administering an isolated population of cells comprising pluripotent stem cell-derived nociceptive neurons expressing CD200^{high} to a joint of the subject.

Provided herein are also methods of treating osteoarthritis or a related condition in a subject thereof, wherein the method comprises administering an isolated population of cells. In some embodiments, the isolated population of cells comprises pluripotent stem cells. In some embodiments, the pluripotent stem cells are human pluripotent stem cells. In some embodiments, the pluripotent stem cells highly express CD200. In some embodiments, the method of treating osteoarthritis or a related condition in a subject thereof comprises administering an isolated population of cells to a joint of the subject. In some embodiments, the joint of the subject is a knee joint. In some embodiments, the method of treating osteoarthritis or a related condition in a subject thereof comprises administering an isolated population of cells comprising pluripotent stem cell-derived nociceptive neurons expressing CD200^{low} to a joint of the subject.

Provided herein are also methods of promoting healing of injured tissues in a subject thereof, wherein the method comprises administering an isolated population of cells. In some embodiments, the isolated population of cells comprises pluripotent stem cells. In some embodiments, the pluripotent stem cells are human pluripotent stem cells. In some embodiments, the pluripotent stem cells highly express CD200. In some embodiments, the method of promoting healing of injured tissues condition in a subject thereof comprises administering an isolated population of cells to a joint of the subject. In some embodiments, the joint of the subject is a knee joint. In some embodiments, the method of promoting healing of injured tissues in a subject thereof comprises administering an isolated population of cells comprising pluripotent stem cell-derived nociceptive neurons expressing CD200^{high} to a joint of the subject.

In some embodiments, the subject may be a neonate, a juvenile, or an adult. Of particular interest are mammalian subjects. Mammalian species that may be treated with the present methods include canines and felines; equines; bovines; ovines; etc. and primates, particularly humans. Animal models, particularly small mammals (e.g. mice, rats, guinea pigs, hamsters, rabbits, etc.) may be used for experimental investigations.

Administration of the pluripotent stem cells described herein, populations thereof, and compositions thereof can occur by injection, irrigation, inhalation, consumption, electro-osmosis, hemodialysis, iontophoresis, and other methods known in the art. In some embodiments, administration route is local or systemic. In some embodiments, administration route is intraarterial, intracranial, intradermal, intraduodenal, intramammary, intrameningeal, intraperitoneal, intrathecal, intratumoral, intravenous, intravitreal, ophthalmic, parenteral, spinal, subcutaneous, ureteral, urethral, vaginal, intraarticular, or intrauterine. In some embodiments, the administration route is intraarticular. In some embodiments, the administration is an intraarticular injection. In some embodiments, the administration is an intraarticular injection into a joint. In some embodiments, the joint comprises temporomandibular joint (jaw), shoulder (glenohumeral), elbow, wrist (radiocarpal), hip, knee, and ankle joints. In some embodiments, the joint comprises a knee joint.

In some embodiments, the administration route is by infusion (e.g., continuous or bolus). Examples of methods for local administration, that is, delivery to the site of injury or disease, include through an Ommaya reservoir, e.g. for intrathecal delivery (See e.g., U.S. Pat. Nos. 5,222,982 and 5,385,582, incorporated herein by reference); by bolus injection, e.g. by a syringe, e.g. into a joint; by continuous infusion, e.g. by cannulation, such as with convection (See e.g., US Patent Application Publication No. 2007-0254842, incorporated herein by reference); or by implanting a device upon which the cells have been reversibly affixed (see e.g. US Patent Application Publication Nos. 2008-0081064 and 2009-0196903, incorporated herein by reference). In some embodiments, the administration route is by topical administration or direct injection. In some embodiments, the modified pluripotent cells described herein may be provided to the subject alone or with a suitable substrate or matrix, e.g. to support their growth and/or organization in the tissue to which they are being transplanted.

In some embodiments, at least 1×10³ cells are administered to a subject. In some embodiments, at least 5×10³ cells, 1×10⁴ cells, 5×10⁴ cells, 1×10⁵ cells, 5×10⁵ cells, 1×10⁶, 2×10⁶, 3×10⁶, 4×10⁶, 5×10⁶, 1×10⁷, 1×10⁸, 5×10⁸, 1×10⁹, 5×10⁹, 1×10¹⁰, 5×10¹⁰, 1×10¹¹, 5×10¹¹, 1×10¹², 5×10¹², or more cells are administered to a subject. In some embodiments, between about 1×10⁷ and about 1×10¹² cells are administered to a subject. In some embodiments, between about 1×10⁸ and about 1×10¹² cells are administered to a subject. In some embodiments, between about 1×10⁹ and about 1×10¹² cells are administered to a subject. In some embodiments, between about 1×10¹⁰ and about 1×10¹² cells are administered to a subject. In some embodiments, between about 1×10¹¹ and about 1×10¹² cells are administered to a subject. In some embodiments, between about 1×10⁷ and about 1×10¹¹ cells are administered to a subject. In some embodiments, between about 1×10⁷ and about 1×10¹⁰ cells are administered to a subject. In some embodiments, between about 1×10⁷ and about 1×10⁹ cells are administered to a subject. In some embodiments, between about 1×10⁷ and about 1×10⁸ cells are administered to a subject. The number of administrations of treatment to a subject may vary. In some embodiments, introducing the modified pluripotent stem cells into the subject may be a one-time event. In some embodiments, such treatment may require an on-going series of repeated treatments. In some embodiments, multiple administrations of the pluripotent stem cells may be required before an effect is observed. The exact protocols depend upon the disease or condition, the stage of the disease and parameters of the individual subject being treated.

In some embodiments, the gene-regulating systems described herein are employed to modify cellular DNA or RNA in vivo, such as for gene therapy or for biological research. In such embodiments, a gene-regulating system may be administered directly to the subject, such as by the methods described supra. In some embodiments, the gene-regulating systems described herein are employed for the ex vivo or in vitro modification of a population of pluripotent stem cells. In such embodiments, the gene-regulating systems described herein are administered to a sample comprising pluripotent stem cells.

In some embodiments, the pluripotent stem cells described herein are administered to a subject. In some embodiments, the pluripotent stems cells described herein are administered to a subject are autologous pluripotent stem cells. The term “autologous” in this context refers to cells that have been derived from the same subject to which they are administered. For example, pluripotent stem cells may be obtained from a subject, modified ex vivo according to the methods described herein, and then administered to the same subject in order to treat a disease. In such embodiments, the cells administered to the subject are autologous pluripotent stem cells. In some embodiments, the modified pluripotent stem cells, or compositions thereof, administered to a subject are allogenic pluripotent stems cells. The term “allogenic” in this context refers to cells that have been derived from one subject and are administered to another subject. For example, pluripotent stem cells may be obtained from a first subject, modified ex vivo according to the methods described herein and then administered to a second subject in order to treat a disease. In such embodiments, the cells administered to the subject are allogenic pluripotent stem cells.

In some embodiments, the pluripotent stem cells described herein are administered to a subject in order to treat a disease. In some embodiments, treatment comprises delivering an effective amount of a population of cells (e.g., a population of modified pluripotent stem cells) or composition thereof to a subject in need thereof. In some embodiments, treating refers to the treatment of a disease in a mammal, e.g., in a human, including (a) inhibiting the disease, i.e., arresting disease development or preventing disease progression; (b) relieving the disease, i.e., causing regression of the disease state or relieving one or more symptoms of the disease; and (c) curing the disease, i.e., remission of one or more disease symptoms. In some embodiments, treatment may refer to a short-term (e.g., temporary and/or acute) and/or a long-term (e.g., sustained) reduction in one or more disease symptoms. In some embodiments, treatment results in an improvement or remediation of the symptoms of the disease. The improvement is an observable or measurable improvement, or may be an improvement in the general feeling of well-being of the subject.

The effective amount of a pluripotent stem cell population administered to a particular subject will depend on a variety of factors, several of which will differ from patient to patient including the disorder being treated and the severity of the disorder; activity of the specific agent(s) employed; the age, body weight, general health, sex and diet of the patient; the timing of administration, route of administration; the duration of the treatment; drugs used in combination; the judgment of the prescribing physician; and like factors known in the medical arts.

In some embodiments, the subject is administered an effective amount of a population of pluripotent stem cells to treat pain. In some embodiments, pain comprises acute pain, chronic pain, nociceptive pain (e.g., somatic or visceral), neuropathic pain, inflammatory pain, and functional or psychogenic pain. In some embodiments, the treated subject exhibits a reduction in pain by at least about 10%, at least about 20%, at least about 30%, at least about 40%, at least about 50%, at least about 60%, at least about 70%, at least about 80%, at least about 90%, or 100% in comparison to the subject prior to treatment or an untreated subject. In some embodiments, the treated subject exhibits at least 1, 2, 3, 4, 5, 6, 7, 8, 9, 10, 20, 30, 40, 50, 60, 70, 80, 90, 100, or more fold decrease in pain in comparison to the subject prior to treatment or an untreated subject.

In some embodiments, the subject is administered an effective amount of a population of pluripotent stem cells to treat or prevent osteoarthritis. In some embodiments, osteoarthritis comprises primary or secondary osteoarthritis. In some embodiments, the osteoarthritis comprises knee, hip, hand, spine, shoulder, or temporomandibular osteoarthritis. In some embodiments, osteoarthritis is localized, generalized or erosive.

In some embodiments, the subject is administered an effective amount of a population of pluripotent stem cells to reduce symptoms related to osteoarthritis. In some embodiments, the symptoms comprise joint pain, stiffness, swelling, reduced range of motion, joint tenderness, gating or cracking sensation, bone spurs, or a combination thereof. In some embodiments, the treated subject exhibits a reduction in symptoms associated with osteoarthritis by at least about 10%, at least about 20%, at least about 30%, at least about 40%, at least about 50%, at least about 60%, at least about 70%, at least about 80%, at least about 90%, or 100% in comparison to the subject prior to treatment or an untreated subject. In some embodiments, the treated subject exhibits at least 1, 2, 3, 4, 5, 6, 7, 8, 9, 10, 20, 30, 40, 50, 60, 70, 80, 90, 100, or more fold decrease in symptoms associated with osteoarthritis in comparison to the subject prior to treatment or an untreated subject.

In some embodiments, the administration of the pluripotent stem cells to a subject reduced the release of pro-inflammatory mediators. In some embodiments, the pluripotent stem cells administered modulate the neuro-immune axis. In some embodiments, the treated subject exhibits a reduction in release of pro-inflammatory immune mediators by at least about 10%, at least about 20%, at least about 30%, at least about 40%, at least about 50%, at least about 60%, at least about 70%, at least about 80%, at least about 90%, or 100% in comparison to the subject prior to treatment or an untreated subject. In some embodiments, the treated subject exhibits at least 1, 2, 3, 4, 5, 6, 7, 8, 9, 10, 20, 30, 40, 50, 60, 70, 80, 90, 100, or more fold decrease in pro-inflammatory mediators in comparison to the subject prior to treatment or an untreated subject.

In some embodiments, the subject is administered an effective amount of a population of pluripotent stem cells to improve bone and cartilage repair. In some embodiments, the treated subject exhibits an increased bone and cartilage tissue repair by at least about 10%, at least about 20%, at least about 30%, at least about 40%, at least about 50%, at least about 60%, at least about 70%, at least about 80%, at least about 90%, or 100% in comparison to the subject prior to treatment or an untreated subject. In some embodiments, the treated subject exhibits at least 1, 2, 3, 4, 5, 6, 7, 8, 9, 10, 20, 30, 40, 50, 60, 70, 80, 90, 100, or more fold increase in bone and cartilage repair in comparison to the subject prior to treatment or an untreated subject.

In some embodiments, the treated subject exhibits a reduction in mechanical hypersensitivity by at least about 10%, at least about 20%, at least about 30%, at least about 40%, at least about 50%, at least about 60%, at least about 70%, at least about 80%, at least about 90%, or 100% in comparison to the subject prior to treatment or an untreated subject.

In some embodiments, the treated subject exhibits a reduction in expression of matrix metalloproteinase 13 (MMP13) by at least about 10%, at least about 20%, at least about 30%, at least about 40%, at least about 50%, at least about 60%, at least about 70%, at least about 80%, at least about 90%, or 100% in comparison to the subject prior to treatment or an untreated subject.

In some embodiments, the pluripotent stem and gene-regulating systems described herein may be used in the treatment of pain, osteoarthritis or osteoarthritis related conditions.

Certain embodiments may provide, for example, a composition for or method of treating a subject, comprising: administering a gene editing composition to the subject. In certain embodiments, for example, the administering may comprise injecting the gene editing composition into the subject. In certain embodiments, for example, the injecting may be intrathecal administration. In certain embodiments, for example, the injecting may be injecting the gene editing composition into a location identified as a location exhibiting pain (or proximate to such location) in the subject. In certain embodiments, for example, the administering may comprise administering to a joint of the subject. In certain embodiments, for example, the joint may be a knee joint.

In certain embodiments, for example, the gene editing composition may comprise a KCNQ2 gene editor. In certain embodiments, for example, the gene editing composition may comprise a SCN9A gene editor. In certain embodiments, for example, the gene editing composition may comprise a MRGPRX1 gene editor. In certain embodiments, for example, the gene editing composition may comprise a TRPV1 gene editor.

In certain embodiments, for example, the method may be associated with a measure of reduction in pain. In certain embodiments, for example, the reduction in pain may be a reduction of at least or about 5%, 10%, 10%, 15%, 20%, 25%, 30%, 35%, 40%, 50%, 60%, 70%, 80%, 90%, 95%, or 100% or any ranges within such percentages. In certain embodiments, for example, the reduction in the measure of pain may be a reduction in the range of between 5% and 90%, for example the reduction may be in the range of between 10% and 75%, in the range of between 10% and 50%, in the range of between 10% and 25%, in the range of between 25% and 75%, in the range of between 25% and 50%, or the reduction in the measure of pain may be a reduction in the range of between 30% and 60%.

In certain embodiments, for example, the measure of reduction in pain may be a measure of pain in the treated subject (for example a pain score such as a verbal or written subjective pain score). In certain embodiments, for example, the measure of reduction in pain may be based at least on a measurement taken after treatment compared to a measurement taken prior to treatment. In certain embodiments, for example, the measure of reduction in pain may be based on a trend of at least three measurements. In certain embodiments, for example, the measure of reduction in pain may be a result of an animal study (for example a study in humans, a study in mice, etc.). In certain embodiments, for example, the measure of reduction in pain may be based on a comparison between a group having a condition that receives the gene editing composition and a control group that does not have the condition. In certain embodiments, for example, the measure of reduction in pain may be based on a comparison between a group having a condition that receives the gene editing composition and a control group that receives a nontargeted gene editing composition.

In certain embodiments, for example, the measure of reduction in pain may comprise a Rotarod test. In certain embodiments, for example, the measure of reduction in pain may comprise a Hargreaves test. In certain embodiments, for example, the measure of reduction in pain may comprise a Von Frey test. In certain embodiments, for example, the measure of reduction in pain may comprise one or more electrophysiology measurements.

In certain embodiments, for example, the composition and/or method may be associated with a change in a measure of itch. In certain embodiments, for example, the change in the measure of itch may be a change of less than or about 30%, 25, 20, 15, 10, 5, 2, or 1% relative to control. In certain embodiments, for example, the change in the measure of itch may be a statistically insignificant change compared to control.

In certain embodiments, for example, the measure of itch may be a measure of itch in the treated subject (for example a verbal or written subjective score). In certain embodiments, for example, the measure of itch may be based at least on a measurement taken after treatment compared to a measurement taken prior to treatment. In certain embodiments, for example, the measure of itch may be based on a trend of at least three measurements. In certain embodiments, for example, the measure of itch may be a result of an animal study (for example a study in humans, a study in mice, etc.). In certain embodiments, for example, the measure of itch may be based on a comparison between a group having a condition that receives the gene editing composition and a control group that does not have the condition. In certain embodiments, for example, the measure of itch may be based on a comparison between a group having a condition that receives the gene editing composition and a control group that receives a nontargeted gene editing composition.

In certain embodiments, for example, the measure of itch may comprise measurement of itch-like biting. In certain embodiments, for example, the measure of itch may comprise measurement of BAM8-22 induced itch-like biting.

In certain embodiments, for example, the subject may be a human. In certain embodiments, for example, the subject may be a domesticated animal. In certain embodiments, for example, the domesticated animal may be a pet. In certain embodiments, for example, the, domesticated animal may be a working animal. In certain embodiments, for example, the domesticated animal may be a dog. In certain embodiments, for example, the domesticated animal may be a cat. In certain embodiments, for example, the animal may be a feral cat (for example a barn cat). In certain embodiments, for example, the domesticated animal may be livestock. In certain embodiments, for example, the livestock may be a cow. In certain embodiments, for example, the livestock may be a sheep. In certain embodiments, for example, the livestock may be a goat. In certain embodiments, for example, the livestock may be a pig. In certain embodiments, for example, the livestock may be a chicken. In certain embodiments, for example, the livestock may be a horse.

In certain embodiments, for example, the pain may comprise chronic pain. In certain embodiments, for example, the pain may comprise nociceptive pain. In certain embodiments, for example, the pain may comprise pruriceptive pain.

Certain embodiments may provide, for example, compositions for and methods of treating a subject to reduce pain while minimizing a change in itch sensation comprising: administering a gene editing composition to the subject. In certain embodiments, for example, the subject may exhibit at least a 10% reduction in pain and less than a 10% change in itch, for example the subject may exhibit at least a 20% reduction in pain and less than a 10% change in itch, at least a 30% reduction in pain and less than a 10% change in itch, at least a 50% reduction in pain and less than a 10% change in itch, at least a 25% reduction in pain and less than a 5% change in itch, or the subject may exhibit at least a 15% reduction in pain and less than a 5% change in itch.

### NUMBERED EMBODIMENTS

Notwithstanding the appended claims, the disclosure sets forth the following numbered embodiments:

Embodiment 1. A method of producing a population of mature human pluripotent stem cell-derived sensory neurons (hPSC-SNs) expressing a target gene associated with at least one of nociceptive pain, chronic pain, pruriception and a nociceptive- or pruriceptive-mediated condition, the method comprising: introducing into a population of human pluripotent stem cells (hPSCs) a composition comprising: at least one site-directed nuclease targeting a site within the target gene, and at least one nucleic acid comprising a nucleotide sequence encoding at least one screenable, selectable marker that is flanked by (i) a nucleotide sequence homologous with a region located upstream of the target site within the target gene and (ii) a nucleotide sequence homologous with a region located downstream of the target site within the target gene, wherein the target site is located downstream of the open reading frame of the target gene, and wherein the site-directed nuclease cleaves the target site of the target gene and the nucleic acid encoding the screenable, selectable marker is inserted at the target site; covering said population of hPSCs under an extracellular matrix comprising at least one neuronal differentiation driver to produce a sensory committed neural crest population; contacting said sensory committed neural crest population with at least one neuronal differentiation driver and at least one neurotrophic factor to produce a population of early sensory neurons (SNs); and contacting said population of early SNs with at least one neurotrophic factor to produce a population of mature hPSC-SNs expressing at least one of SN marker or one pan neuronal marker; isolating the cells expressing the at least one screenable, selectable marker, wherein said population of mature hPSC-SNs expressed said one target gene associated with nociceptive pain, chronic pain, pruriception a nociceptive- or pruriceptive-mediated condition, and wherein said population of mature hPSC-SNs responds to a nociceptive and pruriceptive stimulus.

Embodiment 2. The method of embodiment 1, wherein the target gene is SCN9A, TRPV1, MRGPRX1, or KCNQ2.

Embodiment 3. The method of embodiment 2, wherein the site-directed nuclease is a CRISPR system comprising a ribonuclear protein (RNP) comprising CRISPR nuclease complexed with a guide RNA (gRNA) or a single guide RNA (sgRNA).

Embodiment 4. The method of embodiment 1 or 2, wherein CRISPR nuclease is a Type II Cas9 nuclease or a Type V Cfpl nuclease, and the CRISPR nuclease is linked to at least one nuclear localization signal.

Embodiment 5. The method according to any one of embodiments 1 to 4, wherein the at least one screenable, selectable marker is a fluorescent reporting protein, an antibiotic resistance protein, or a combination thereof.

Embodiment 6. The method according to embodiment 5, wherein the fluorescent reporting protein is selected from green fluorescent protein (GFP), yellow fluorescent protein (YFP), red fluorescent protein (RFP), lacZ, firefly Rennila protein, luciferase, red cyan protein, and yellow cyan protein.

Embodiment 7. The method of embodiment 6, wherein the fluorescent reporting protein is selected from green fluorescent protein (GFP).

Embodiment 8. The method of embodiment 5, wherein the antibiotic resistance protein is selected from hygromycin, neomycin, zeocin, and puromycin.

Embodiment 9. The method of embodiment 8, wherein the antibiotic resistance protein is puromycin.

Embodiment 10. The method according to any one of embodiments 2 to 9, wherein the target gene is SEQ ID NO: 1, the nucleic acid encoding the at least one screenable, selectable marker comprises SEQ ID NO: 2, and the gRNA comprises a spacer sequence corresponding to a target sequence consisting of SEQ ID NO: 1

Embodiment 11. The method according to any one of embodiments 2 to 9, wherein the target gene is SEQ ID NO: 3, the nucleic acid encoding the at least one screenable, selectable marker comprises SEQ ID NO: 2, and the gRNA comprises a spacer sequence corresponding to a target sequence consisting of SEQ ID NO: 3.

Embodiment 12. The method of embodiment 10, wherein the hPSC-SNs are enriched in voltage gated potassium channel genes, active transporter genes, GPCR receptors, or any combination thereof.

Embodiment 13. The method of embodiment 11, wherein the hPSC-SNs are enriched in voltage gated potassium channel genes, active transporter genes, GPCR receptors, or any combination thereof.

Embodiment 14. The method of embodiments 12 to 13, wherein the voltage gated potassium channel genes comprise KCNQ2 and KCNG2.

Embodiment 15. The method according to embodiment 12 to 13, wherein the active transporter genes comprise SLC10A4 and ATP2B4.

Embodiment 16. The method according to embodiment 12 to 13, wherein the GPCR receptors comprise ADCYAP1R1.

Embodiment 17. The method according to any one of embodiments 2 to 9, wherein the target gene is SEQ ID NO: 4, the nucleic acid encoding the at least one screenable, selectable marker comprises SEQ ID NO: 2, and the gRNA comprises a spacer sequence corresponding to a target sequence consisting of SEQ ID NO: 4.

Embodiment 18. The method according to embodiment 17, wherein hPSC-SNs are enriched in voltage gated calcium channel genes, GPCR receptors, or any combination thereof.

Embodiment 19. The method according to embodiment 18, wherein the voltage gated calcium channel genes comprise CACNG1, CACNA1S, CACNG8, and CACNA2D3.

Embodiment 20. The method according to embodiment 18, wherein the GPCR receptors comprise PRNP, DNM3, UTS2R, HTR2C, HTR1E, HTR2A HTR1D, and NPY1R.

Embodiment 21. The method according to any one of embodiments 1 to 20 wherein the composition further comprises at least one agent capable of favoring the homology-directed repair (HDR) pathway over the non-homologous end-joining (NHEJ) pathway.

Embodiment 22. The method according to embodiment 21, wherein the agent is an HDR activator or NHEJ inhibitor.

Embodiment 23. The method according to embodiment 21, wherein the HDR activator is selected from the group comprising: RAD51, RAD52, DMC 1, CtIP, or any combination thereof.

Embodiment 24. The method according to embodiment 21, wherein the NHEJ inhibitor selected from E1B55K, E4orf6, 53BP1(DM), Rif, p53, or any combination thereof.

Embodiment 25. A population of hPSC-SNs produced by the method according to any one of embodiments 1-24.

Embodiment 26. The method of embodiment 1, wherein the target gene is associated with nociceptive pain.

Embodiment 27. The method of embodiment 1, wherein the target gene is associated with chronic pain.

Embodiment 28. A population of hPSC-SNs produced by the method according to any one of embodiments 25-27.

Embodiment 29. A method of altering the expression or function of at least one target gene associated with nociceptive pain, chronic pain, pruriception and a nociceptive/pruriceptive condition in a sensory neuron (SN), the method comprising: introducing into SN a composition comprising: at least one site-directed nuclease targeting a site within the target gene, and optionally, at least one nucleic acid comprising a nucleotide sequence encoding a gene expression altering sequence that is flanked by (i) a nucleotide sequence homologous with a region located upstream of the target site within the target gene and (ii) a nucleotide sequence homologous with a region located downstream of the target site within the target gene, and wherein the site-directed nuclease cleaves the target site of the target gene and the nucleic acid encoding the gene expression altering sequence is inserted at the target site; wherein the expression or function of said at least one target gene is altered.

Embodiment 30. The method of embodiment 29, wherein the site-directed nuclease is a CRISPR system comprising a ribonuclear protein comprising CRISPR nuclease complexed with a guide RNA (gRNA) or a single guide RNA (sgRNA).

Embodiment 31. The method of embodiment 30, wherein CRISPR nuclease is a Type II Cas9 nuclease or a Type V Cfpl nuclease, and the CRISPR nuclease is linked to at least one nuclear localization signal.

Embodiment 32. The method of embodiment 29, wherein the composition is a vector composition comprising: (a) a polynucleotide sequence encoding a type of Cas9 protein variant or a fusion protein comprising at least one variant from of Cas9 protein; (b) a polynucleotide sequence encoding at least one guide RNA (gRNA) or a single guide RNA (sgRNA); (c) one or more promoters, each promoter operably linked to the polynucleotide sequence encoding the at least one gRNA and the polynucleotide sequence encoding the Cas9 protein or fusion protein; (d) optionally, a polynucleotide sequence encoding at least one gene expression altering sequence that is flanked by (i) a nucleotide sequence homologous with a region located upstream of the target site within the target gene and (ii) a nucleotide sequence homologous with a region located downstream of the target site within the target gene.

Embodiment 33. The method of embodiment 32, wherein variants of Cas9 protein comprise SpCas9, SaCas9.

Embodiment 34. The method of embodiment 32, wherein the vector is a viral vector.

Embodiment 35. The method according to embodiment 34, wherein the viral vector is an Adeno-associated virus (AAV) vector.

Embodiment 36. The method of embodiment 35, wherein the AAV vector is an AAV8 vector, an AAV1 vector, an AAV6.2 vector, an AAVrh74 vector, an AAV9 vector, or an AAV PUP.s.

Embodiment 37. The method according to any one of embodiments 32-36 wherein the vector composition comprises a single vector comprising: (a) a polynucleotide sequence encoding a variant from the class of Cas9 proteins or a fusion protein comprising a variant from the class of Cas9 proteins; (b) a polynucleotide sequence encoding at least one guide RNA (gRNA) or a single guide RNA (sgRNA); (c) one or more promoters, each promoter operably linked to the polynucleotide sequence encoding the at least one gRNA and the polynucleotide sequence encoding the Cas9 protein or fusion protein; (d) optionally, a polynucleotide sequence encoding at least one gene expression altering sequence that is flanked by (i) a nucleotide sequence homologous with a region located upstream of the target site within the target gene and (ii) a nucleotide sequence homologous with a region located downstream of the target site within the target gene.

Embodiment 38. The method of any one of embodiments 32-37, wherein the vector composition comprises two or more vectors comprising (a) a polynucleotide sequence encoding a type of Cas9 variant protein n or a fusion protein comprising the Cas9 protein; (b) a polynucleotide sequence encoding at least one guide RNA (gRNA) or a single guide RNA (sgRNA); (c) one or more promoters, each promoter operably linked to the polynucleotide sequence encoding the at least one gRNA and the polynucleotide sequence encoding the Cas9 protein or fusion protein; (d) optionally, a polynucleotide sequence encoding at least one gene expression altering sequence that is flanked by (i) a nucleotide sequence homologous with a region located upstream of the target site within the target gene and (ii) a nucleotide sequence homologous with a region located downstream of the target site within the target gene.

Embodiment 39. The method of any one of embodiments 32-37, wherein the vector composition comprises: a first vector comprising (i) a polynucleotide sequence encoding a type of Cas9 protein variant or a fusion protein comprising the variant of Cas9 protein; and (ii) a promoter operably linked to the polynucleotide sequence encoding the variant of Cas9 protein or a fusion protein comprising the variant of Cas9 protein a second vector comprising (i) a polynucleotide sequence encoding at least one guide RNA (gRNA) or a single guide RNA (sgRNA); (ii) a promoter operably linked to the polynucleotide sequence encoding the at least one guide RNA (gRNA) or a single guide RNA (sgRNA) and, optionally, (iii) a polynucleotide sequence encoding at least one gene expression altering sequence that is flanked by (a) a nucleotide sequence homologous with a region located upstream of the target site within the target gene and (b) a nucleotide sequence homologous with a region located downstream of the target site within the target gene.

Embodiment 40. The method according to any one of embodiments 1-39, wherein the dual AAV-mediated delivery comprises TRPV1 sgRNA and SaCas9, consisting of SEQ ID NOs: 5, 6, and 7.

Embodiment 41. The method according to any one of embodiments 1-40, wherein the dual AAV-mediated delivery comprises SCN9A sgRNA and SaCas9, consisting of SEQ ID NOs: 5, 8, and 9.

Embodiment 42. The method according to any one of embodiments 29-39, wherein the expression of the target gene is inhibited.

Embodiment 43. The method according to any one of embodiments 29-42, wherein the target gene is from SCN9A, TRPV1, MRGPRX1, KCNQ2 or any combination thereof.

Embodiment 44. The method according to any one of embodiments 29-42, wherein the target gene is SCN9A and the gRNA comprises a spacer sequence corresponding to a target sequence consisting of SEQ ID NO: 1.

Embodiment 45. The method according to any one of embodiments 25-43, wherein the target gene is TRPV1 and the gRNA comprises a spacer sequence corresponding to a target sequence consisting of SEQ ID NO.3.

Embodiment 46. The method according to any one of embodiments 25-43, wherein the target gene is KCNQ2, the nucleic acid encoding the gene expression altering sequence comprising SEQ ID NO: 10, and the gRNA comprises a spacer sequence corresponding to a target sequence consisting of SEQ ID NO: 10.

Embodiment 47. The method of embodiment 46, wherein KCNQ2 function is altered by introducing the mutation c.2188A>G, p.Thr730Ala set forth in SEQ ID NO: 10.

Embodiment 48. The method of embodiments 46 or 47, wherein the composition administered to SN further comprises a second gRNA comprising a spacer sequence corresponding to a target sequence consisting of SEQ ID NO: 10.

Embodiment 49. The method according to embodiment 48, wherein KCNQ2 function is altered by introducing a CAC (histidine) to CGC (arginine) mutation.

Embodiment 50. The method according to any one of embodiments 46-49, wherein the composition administered to SN further comprises at least one nucleic acid comprising a nucleotide sequence encoding a base editor operably linked to a promoter, wherein the base editor comprises a nucleic acid programmable DNA binding protein (napDNAbp) domain and a deaminase domain.

Embodiment 51. The method of embodiment 50, wherein the base editor further comprises one or more nuclear localization sequences (NLS).

Embodiment 52. The method of embodiment 50 or 51, wherein the base editor is ABE8e, ABE8e(V106W), ABE9, ABE20, ABE7.10, or a variant thereof.

Embodiment 53. The method according to any one of embodiments 50-52, wherein the base editor is SaKKH-ABE8e, SauriCas9-ABE8e, CjCas9-ABE8e, Nme2Cas9-ABE8e, or SaCas9-ABE8e, or a variant thereof.

Embodiment 54. The method according to any one of embodiments 50-53, wherein the base editor is SauriCas9-ABE8e.

Embodiment 55. The method according to any one of embodiments 29-54, wherein the sensory neurons expressing mutation have diminished and protracted Ca²⁺ response to KC1 stimulation as compared to neurons expressing wild type KCNQ2.

Embodiment 56. The method according to any one of embodiments 29-55, wherein the sensory neurons is produced according to the method of embodiment 1.

Embodiment 57. The method according to any one of embodiments 29-55, wherein the sensory neurons is produced according to the method of embodiments 29 or 30.

Embodiment 58. A population of sensory neurons produced by the method according to any one of embodiments 29-57.

Embodiment 59. A method of producing an enriched population of mature human pluripotent stem cell-derived nociceptive sensory neurons (hPSC-NSNs) expressing CD200 gene (CD200+), the method comprising: contacting hPSC-NSNs produced by the method of embodiment 1 with a CD200-specific antibody; and separating the antibody-bound cells from the non-bound cells in order to obtain the purified cells.

Embodiment 60. The method according to embodiment 59, wherein the CD200-specific antibody is OX-104.

Embodiment 61. The method of embodiment 59, wherein the separation step is carried out by means of MACS or FACS.

Embodiment 62. The method according to embodiment 61, wherein the separation step is carried out by means of FACS.

Embodiment 63. The method according to any one of embodiments 59-62, wherein the enriched CD200+ hPSC-NSNs population is also expresses MiR-24 miRNA.

Embodiment 64. A method of treating nociceptive pain, chronic pain, pruriception and a nociceptive- or pruriceptive-mediated condition in a subject in need thereof, the method comprising administering to a subject the population of sensory neurons having the function of KCNQ2 altered according to methods of any one of embodiments 46-54 into a subject.

Embodiment 65. A method of treating nociceptive pain, chronic pain, pruriception and a nociceptive- or pruriceptive-mediated condition in a subject in need thereof, the method comprising administering to a subject the population of CD200+ sensory neurons of any one of embodiments 59-64.

Embodiment 66. The method according to embodiments 64 or 65, wherein the administering is by implanting the population of sensory neurons onto one or more affected locations of the subject.

Embodiment 67. The method according to embodiment 59, wherein the population of sensory neurons is implanted at the one or more sites of nociceptive pain and chronic pain.

Embodiment 68. The method according to any one of embodiments 64-67, wherein the population of sensory neurons is autologous to the subject.

Embodiment 69. The method according to any one of embodiments 64-67, wherein the population of sensory neurons is allogeneic to the subject.

Embodiment 70. The method according to any one of embodiments 64-69, wherein the nociceptive, chronic, and pruriceptive condition is osteoarthritis.

Embodiment 71. The method according to any one of embodiments 64-69, wherein the nociceptive, chronic, pruriceptive condition is derived from neuroinflammation or neurodegeneration, or a combination thereof.

Embodiment 72. The method according to any one of embodiments 1-39, wherein the dual AAV-mediated delivery comprises KCNQ2, consisting of SEQ ID NOs: 11, 12, and 13.

Embodiment 73. The method according to any one of embodiments 1-39, wherein the dual AAV-mediated delivery comprises KCNQ2, consisting of SEQ ID NOs: 14, 15, and 16.

Embodiment 74. A method for screening for a drug candidate that modulates one or more MRGPRX1, TRPV1 or SCN9A-mediated conditions the method comprising: contacting at least one population of hPSC-SNs expressing one of MRGPRX1, TRPV1 or SCN9A produced according to any one of embodiments 1-24 with a drug candidate; and detecting a response to a nociceptive and pruriceptive stimulus in said at least one population of hPSC-SNs expressing one of MRGPRX1, TRPV1 or SCN9A to thereby select said drug candidate for modulating said response.

Embodiment 75 relates to an isolated population of cells comprising pluripotent stem cell-derived sensory neurons (PSC-SNs) expressing at least one sensory neuron or pan neuronal marker.

Embodiment 76 includes the cells of embodiment 75, wherein the PSC-SNs respond to a nociceptive and pruriceptive stimulus.

Embodiment 77 includes the cells of embodiment 75 or 76, comprising cells expressing TRPV1, SCN9A, MRGPRX1, or KCNQ2.

Embodiment 78 includes the cells of any one of embodiments 75-77, comprising cells expressing CD200^{high}.

Embodiment 79 includes the cells of embodiment 78, wherein at least 40%, at least 45%, at least 50%, at least 55%, at least 60%, at least 65%, at least 70%, at least 75%, at least 80%, at least 85%, at least 90%, at least 95%, at least 99%, or 100% of the cells express CD200^{high}.

Embodiment 80 includes the cells of any one of embodiments 75-79, comprising PSC-SNs derived from an animal or a human (hPSC-SNs).

Embodiment 81 includes the cells of any one of embodiments 75-80, wherein the PSC-SNs comprise pluripotent stem cell-derived nociceptive sensory neurons (PSC-NSNs).

Embodiment 82 relates to a composition comprising the cells of any one of embodiments 75-81, and at least one excipient.

Embodiment 83 relates to a method of treating pain or osteoarthritis, and/or promoting healing of injured tissues in a subject thereof, wherein the method comprises administering the cells of any one of embodiments 75-81 to the subject.

Embodiment 84 includes the method of embodiment 83, wherein the method comprises administering to a joint of the subject.

Embodiment 85 includes the method of embodiment 84, wherein the joint is a knee joint.

Embodiment 86 includes the method of any one of embodiments 83-85, wherein the pain comprises chronic pain, nociceptive pain, or pruriceptive pain.

Embodiment 87 includes the method of any one of embodiments 83-86, wherein the cells are administered to the subject at multiple intervals.

Embodiment 88 includes the method of any one of embodiments 83-87, wherein the subject is an animal or human.

Embodiment 89 includes the method of any one of embodiments 83-88, wherein the treated subject exhibits a reduction in pain by at least about 10%, at least about 20%, at least about 30%, at least about 40%, at least about 50%, at least about 60%, at least about 70%, at least about 80%, at least about 90%, or 100% in comparison to the subject prior to treatment or an untreated subject.

Embodiment 90 includes the method of any one of embodiments 83-89, wherein the treated subject exhibits a reduction in mechanical hypersensitivity by at least about 10%, at least about 20%, at least about 30%, at least about 40%, at least about 50%, at least about 60%, at least about 70%, at least about 80%, at least about 90%, or 100% in comparison to the subject prior to treatment or an untreated subject.

Embodiment 91 includes the method of any one of embodiments 83-90, wherein the treated subject exhibits a reduction in expression of matrix metalloproteinase 13 (MMP13) by at least about 10%, at least about 20%, at least about 30%, at least about 40%, at least about 50%, at least about 60%, at least about 70%, at least about 80%, at least about 90%, or 100% in comparison to the subject prior to treatment or an untreated subject.

Embodiment 92 relates to a composition for base editing target gene mutations, wherein the composition comprises a base editor and a guide RNA (gRNA) targeting at least one gene, wherein the at least one target gene comprises SCN9A, TRPV1, MRGPRX1, or KCNQ2.

Embodiment 93 includes the composition of embodiment 92, wherein the gRNA comprises a sequence selected from any one of SEQ ID NOs: 82-97.

Embodiment 94 includes the composition of embodiment 92, wherein the gRNA comprises a spacer sequence selected from any one of SEQ ID NOs: 17, 22, 27, 32, 35, 38, 41, 44, 47, 50, 53, 56, or 59-81.

Embodiment 95 includes the composition of any one of embodiments 92-94, wherein the base editor comprises ABE8e, ABE8e(V106W), ABE9, ABE20, ABE7.10, SaKKHABE8e, SauriCas9-ABE8e, CjCas9-ABE8e, Nme2Cas9-ABE8e, or SaCas9-ABE8e.

Embodiment 96 includes the composition of any one of embodiments 92-94, wherein the base editor comprises Cas9 endonuclease.

Embodiment 97 includes the composition of embodiment 96, wherein the Cas9 endonuclease comprises Type II Cas9.

Embodiment 98 relates to a method of editing at least one target gene comprising the composition of any one of embodiments 92-97.

Embodiment 99 relates to a method of producing an isolated population of cells, wherein the population of cells comprises cells of any one of embodiments 75-81, the method comprising contacting the cells with a composition for base editing target gene mutations, wherein the composition comprises a system comprising a base editor and a gRNA targeting at least one gene.

Embodiment 100 includes the method of embodiment 99, wherein the cells have altered expression of the at least one target gene.

Embodiment 101 includes the method of embodiment 99 or 100, wherein the least one target gene comprises SCN9A, TRPV1, MRGPRX1, or KCNQ2.

Embodiment 102 includes the method of any one of embodiments 99-101, wherein the gRNA comprises a sequence selected from any one of SEQ ID NOs: 82-97.

Embodiment 103 includes the method of embodiment 99-101, wherein the gRNA comprises a spacer sequence selected from any one of SEQ ID NOs: 17, 22, 27, 32, 35, 38, 41, 44, 47, 50, 53, 56, or 59-81.

Embodiment 104 includes the method of any one of embodiments 99-103, wherein the base editor comprises ABE8e, ABE8e(V106W), ABE9, ABE20, ABE7.10, SaKKHABE8e, SauriCas9-ABE8e, CjCas9-ABE8e, Nme2Cas9-ABE8e, or SaCas9-ABE8e.

Embodiment 105 includes the method of any one of embodiments 99-103, wherein the base editor comprises Cas9 endonuclease.

Embodiment 106 includes the method of embodiment 105, wherein the Cas9 endonuclease comprises Type II Cas9.

Embodiment 107 relates to a composition for base editing KCNQ2 mutations, wherein the composition comprises a system comprising a base editor and a gRNA targeting KCNQ2.

Embodiment 108 includes the composition of embodiment 107, wherein the gRNA comprises a sequence of SEQ ID NO: 83.

Embodiment 109 includes the composition of embodiment 107, wherein the gRNA comprises a spacer sequence selected from any one of SEQ ID NOs: 32, 35, 38, 41, 44, 47, 50, 53, 56, or 59-81.

Embodiment 110 includes the composition of any one of embodiments 107-109, wherein the base editor comprises ABE8e, ABE8e(V106W), ABE9, ABE20, ABE7.10, SaKKHABE8e, SauriCas9-ABE8e, CjCas9-ABE8e, Nme2Cas9-ABE8e, or SaCas9-ABE8e.

Embodiment 111 relates to a method of editing KCNQ2 mutations comprising the composition of any one of embodiments 107-110.

Embodiment 112 includes the method of embodiment 111, wherein KCNQ2 function is restored.

Embodiment 113 includes the method of embodiment 111, wherein the KCNQ2 mutations comprises a p. T730A or c.2188A>G mutation.

Embodiment 114 relates to a composition for silencing expression of TRPV1 and SCN9A, wherein the composition comprises a system comprising a Cas9 endonuclease and a gRNA targeting TRPV1 and SCN9A.

Embodiment 115 includes the composition of embodiment 114, wherein the gRNA comprises a sequence selected from any one of SEQ ID NO: 86, 89-92, or 97.

Embodiment 116 includes the composition of embodiment 114, wherein the gRNA comprises a spacer sequence selected from any one of SEQ ID NOs: 22 or 27.

Embodiment 117 includes the composition of any one of embodiments 114-116, wherein the Cas9 endonuclease comprises Type II Cas9.

Embodiment 118 relates to method of editing TRPV1 or SCN9A comprising the composition of any one of embodiments 114-117.

Embodiment 119 includes the method of embodiment 118, wherein TRPV1 or SCN9A expression is reduced or eliminated.

Embodiment 120 relates to a method of producing an isolated population of cells comprising human pluripotent stem cell-derived nociceptive sensory neurons (hPSC-NSNs) expressing CD200^{high} with altered expression of KCNQ2, TRPV1, or SCN9A.

Embodiment 121 includes the method of embodiment 120, wherein the cells are contacted with a composition for base editing KCNQ2 mutations, wherein the composition comprises a system comprising a base editor and a gRNA targeting KCNQ2.

Embodiment 122 includes the method of embodiment 121, wherein the gRNA comprises a sequence of SEQ ID NO: 83.

Embodiment 123 includes the method of embodiment 121, wherein the gRNA comprises a spacer sequence selected from any one of SEQ ID NOs: 32, 35, 38, 41, 44, 47, 50, 53, 56, or 59-81.

Embodiment 124 includes the method of any one of embodiments 121-123, wherein the base editor comprises ABE8e, ABE8e(V106W), ABE9, ABE20, ABE7.10, SaKKHABE8e, SauriCas9-ABE8e, CjCas9-ABE8e, Nme2Cas9-ABE8e, or SaCas9-ABE8e.

Embodiment 125 includes the method of embodiment 120, wherein the cells are contacted with a composition for silencing expression of TRPV1 and SCN9A, wherein the composition comprises a system comprising a Cas9 endonuclease and a gRNA targeting TRPV1 and SCN9A.

Embodiment 126 includes the method of embodiment 125, wherein the gRNA comprises a sequence of SEQ ID NO: 86, 89-92, or 97.

Embodiment 127 includes the method of embodiment 125, wherein the gRNA comprises a spacer sequence selected from any one of SEQ ID NOs: 22 and 27.

Embodiment 128 includes the method of any one of embodiments 125-127, wherein the Cas9 endonuclease comprises Type II Cas9.

Embodiment 129 relates to a method of treating a subject, comprising: administering a gene editing composition to the subject.

Embodiment 130 includes the composition of any one of embodiments 1-129, wherein the administering comprises injecting the gene editing composition into the subject.

Embodiment 131 includes the composition of any one of embodiments 1-130, wherein the injecting is intrathecal administration.

Embodiment 132 includes the composition of any one of embodiments 1-131, wherein the injecting is injecting the gene editing composition into a location identified as a location exhibiting pain (or proximate to such location) in the subject.

Embodiment 133 includes the composition of any one of embodiments 1-132, wherein the administering comprises administering to a joint of the subject.

Embodiment 134 includes the composition of any one of embodiments 1-133, wherein the join is a knee joint.

Embodiment 135 includes the composition of any one of embodiments 1-134, wherein the gene editing composition comprises a KCNQ2 gene editor.

Embodiment 136 includes the composition of any one of embodiments 1-135, wherein the gene editing composition comprises a SCN9A gene editor.

Embodiment 137 includes the composition of any one of embodiments 1-136, wherein the gene editing composition comprises a MRGPRX1 gene editor.

Embodiment 138 includes the composition of any one of embodiments 1-137, wherein the gene editing composition comprises a TRPV1 gene editor.

Embodiment 139 includes the composition of any one of embodiments 1-138, wherein the method is associated with a measure of reduction in pain.

Embodiment 140 includes the composition of any one of embodiments 1-139, wherein the reduction in pain is a reduction of at least 10%, for example the reduction is at least 20%, the reduction is at least 25%, the reduction is at least 30%, the reduction is at least 30%, the reduction is at least 40%, the reduction is at least 50%, the reduction is at least 60%, the reduction is at least 70%, the reduction is at least 80%, or the reduction in pain is a reduction of at least 90%.

Embodiment 141 includes the composition of any one of embodiments 1-140, wherein the reduction in the measure of pain is a reduction in the range of between 5% and 90%, for example the reduction is in the range of between 10% and 75%, in the range of between 10% and 50%, in the range of between 10% and 25%, in the range of between 25% and 75%, in the range of between 25% and 50%, or the reduction in the measure of pain is a reduction in the range of between 30% and 60%.

Embodiment 142 includes the composition of any one of embodiments 1-141, wherein the measure of reduction in pain is a measure of pain in the treated subject (for example a pain score such as a verbal or written subjective pain score).

Embodiment 143 includes the composition of any one of embodiments 1-142, wherein the measure of reduction in pain is based at least on a measurement taken after treatment compared to a measurement taken prior to treatment.

Embodiment 144 includes the composition of any one of embodiments 1-143, wherein the measure of reduction in pain is based on a trend of at least three measurements.

Embodiment 145 includes the composition of any one of embodiments 1-144, wherein the measure of reduction in pain is a result of an animal study (for example a study in humans, a study in mice, etc.).

Embodiment 146 includes the composition of any one of embodiments 1-145, wherein the measure of reduction in pain is based on a comparison between a group having a condition that receives the gene editing composition and a control group that does not have the condition.

Embodiment 147 includes the composition of any one of embodiments 1-146, wherein the measure of reduction in pain is based on a comparison between a group having a condition that receives the gene editing composition and a control group that receives a nontargeted gene editing composition.

Embodiment 148 includes the composition of any one of embodiments 1-147, wherein the measure of reduction in pain comprises a Rotarod test.

Embodiment 149 includes the composition of any one of embodiments 1-148, wherein the measure of reduction in pain comprises a Hargreaves test.

Embodiment 150 includes the composition of any one of embodiments 1-149, wherein the measure of reduction in pain comprises a Von Frey test.

Embodiment 151 includes the composition of any one of embodiments 1-150, wherein the measure of reduction in pain comprises one or more electrophysiology measurements.

Embodiment 152 includes the composition of any one of embodiments 1-151, wherein the method is associated with a change in a measure of itch.

Embodiment 153 includes the composition of any one of embodiments 1-152, wherein the change in the measure of itch is a change of less than 30%, for example a change of less than 25%, a change of less than 20%, a change of less than 15%, a change of less than 10%, a change of less than 5%, or the change in the measure of itch is a change of less than 2%.

Embodiment 154 includes the composition of any one of embodiments 1-153, wherein the change in the measure of itch is a statistically insignificant change.

Embodiment 155 includes the composition of any one of embodiments 1-154, wherein the measure of itch is a measure of itch in the treated subject (for example a verbal or written subjective score).

Embodiment 156 includes the composition of any one of embodiments 1-155, wherein the measure of itch is based at least on a measurement taken after treatment compared to a measurement taken prior to treatment.

Embodiment 157 includes the composition of any one of embodiments 1-156, wherein the measure of itch is based on a trend of at least three measurements.

Embodiment 158 includes the composition of any one of embodiments 1-157, wherein the measure of itch is a result of an animal study (for example a study in humans, a study in mice, etc.).

Embodiment 159 includes the composition of any one of embodiments 1-158, wherein the measure of itch is based on a comparison between a group having a condition that receives the gene editing composition and a control group that does not have the condition.

Embodiment 160 includes the composition of any one of embodiments 1-159, wherein the measure of itch is based on a comparison between a group having a condition that receives the gene editing composition and a control group that receives a nontargeted gene editing composition.

Embodiment 161 includes the composition of any one of embodiments 1-160, wherein the measure of itch comprises measurement of itch-like biting.

Embodiment 162 includes the composition of any one of embodiments 1-161, wherein the measure of itch comprises measurement of BAM8-22 induced itch-like biting.

Embodiment 163 includes the composition of any one of embodiments 1-162, wherein the subject is a human.

Embodiment 164 includes the composition of any one of embodiments 1-163, wherein the subject is a domesticated animal.

Embodiment 165 includes the composition of any one of embodiments 1-164, wherein the domesticated animal is a pet.

Embodiment 166 includes the composition of any one of embodiments 1-165, wherein the domesticated animal is a working animal.

Embodiment 167 includes the composition of any one of embodiments 1-166, wherein the domesticated animal is a dog.

Embodiment 168 includes the composition of any one of embodiments 1-167, wherein the domesticated animal is a cat.

Embodiment 169 includes the composition of any one of embodiments 1-168, wherein the animal is a feral cat (for example a barn cat).

Embodiment 170 includes the composition of any one of embodiments 1-169, wherein the domesticated animal is livestock.

Embodiment 171 includes the composition of any one of embodiments 1-170, wherein the livestock is a cow.

Embodiment 172 includes the composition of any one of embodiments 1-171, wherein the livestock is a sheep.

Embodiment 173 includes the composition of any one of embodiments 1-172, wherein the livestock is a goat.

Embodiment 174 includes the composition of any one of embodiments 1-173, wherein the livestock is a pig.

Embodiment 175 includes the composition of any one of embodiments 1-174, wherein the livestock is a chicken.

Embodiment 176 includes the composition of any one of embodiments 1-175, wherein the livestock is a horse.

Embodiment 177 includes the composition of any one of embodiments 1-176, wherein the pain comprises chronic pain.

Embodiment 178 includes the composition of any one of embodiments 1-177, wherein the pain comprises nociceptive pain.

Embodiment 179 includes the composition of any one of embodiments 1-178, wherein the pain comprises pruriceptive pain.

Embodiment 180 relates to a method of treating a subject to reduce pain while limiting a change in itch, comprising: administering a gene editing composition to the subject.

Embodiment 181 includes the composition of any one of embodiments 1-180, wherein the subject exhibits at least a 10% reduction in pain and less than a 10% change in itch, for example the subject exhibits at least a 20% reduction in pain and less than a 10% change in itch, at least a 30% reduction in pain and less than a 10% change in itch, at least a 50% reduction in pain and less than a 10% change in itch, at least a 25% reduction in pain and less than a 5% change in itch, or the subject exhibits at least a 15% reduction in pain and less than a 5% change in itch.

### EXAMPLES

### Example 1. Materials and Methods

hPSC Maintenance

Healthy control hESC (H9, WiCell) were cultured using standard protocols. The hESC line was cultured with mouse embryonic fibroblasts (MEFs) (Gibco) pre-plated at 12,000-15,000 cells/cm². hPSC culture medium contained DMEM/F12, 20% knockout serum replacement, 1 mM L-glutamine, 100 μM MEM non-essential amino acids, and 0.1 mM 2-mercaptoethanol. 10 ng/mL of FGF-2 was added after sterile filtration, and cells were fed daily, and passaged weekly, using 6 U/mL dispase or mechanically.

Generation of Knock-In hPSC Lines

The feeder-free hPSCs were dissociated into single cells using Accutase (Innovative Cell Technologies), and 1.5×10⁶ H9 cells were resuspended in nucleofection solution V (Lonza) with 5 g HDR donor plasmid, 2 g i53 plasmid, 0.5 nmol sgRNA, and 0.5 nmol Cas9 nuclease (V3 or HiFi V3) for TRPV1::GFP, MRGPRX1::GFP and SCN9A::GFP (FIG. 12). The RNP complex for each reporter line was assembled immediately prior to nucleofection. 1.5×106 H9 cells were nucleofected with 5 g HDR donor plasmid, 2 g i53 plasmid, 0.5 nmol sgRNA, and 0.5 nmol Cas9 nuclease (V3 or HiFi V3) using Amaxa Lonza Nucleofector II system. Nucleofection was performed with Nucleofector™ II according to the manufacturer's instruction (using the B-16 program, Lonza). The nucleofected cell suspension was subsequently plated on puromycin resistant MEFs (DR4, Global Stem) in hESC medium with 10 μM Y-27632. Three days after nucleofection, the cells that had undergone homologous recombination were selected by adding 0.5 g/ml of puromycin to hESC medium for two days.

Sensory Neuron Differentiation from hESCs

hPSCs were dissociated into single cells using TrypLE™, and plated on a 0.1% gelatin-coated dish for 10 minutes to remove MEFs. Non-adherent cells (mostly hPSCs) were collected, and plated on a 1% geltrex-coated dish (1 hour coating), at a density of 4.6×10⁴ cells per well of a 24-well plate, in the presence of filtered MEF-conditioned KSR media containing 10 ng/ml of FGF-2 (R&D systems) and 10 μM of Y-27632 (Cayman Chemical) (day −1). From the next day (day 0) (70% of confluence), to initiate differentiation, the medium was aspirated and KSR medium containing 10 μM SB-431542 and 500 nM LDN-193189 was added. On day 2 the medium was changed to KSR medium containing 10 μM SB-431542 and 500 nM LDN-193189.

On day 4 medium was changed to KSR/N2 (3:1) medium containing 10 μM SB-431542, 500 nM LDN-193189, and 10 μM DAPT (final concentrations are for the combined KSR/N2 mixture). On day 6, the medium was changed to KSR/N2 (1:1) medium containing 10 μM SB-431542, 500 nM LDN-193189, and 10 μM DAPT. On day 8, the medium was changed to KSR/N2 (1:3) medium containing 10 μM SB-431542, 500 nM LDN-193189, and 10 μM DAPT. On day 10, the medium was changed to N2 medium containing 10 μM SB-431542, 500 nM LDN-193189, and 10 μM DAPT. On day 12, neural crest cells were dissociated into single cells using TrypLE™ and plated on poly-lysine/laminin/fibronectin coated 12-well plates with a density of 2×10⁴-7×10⁴ cells per 5 μl media. 5 drops were added to each well 12-well plates. Cells were incubated in 5 ul droplets for 10 minutes at 37° C. to fully attach. NB media containing 10 ng/ml of FGF-2 and 10 μM of Y-27632 was then gently added to each well through the wall. On day 14, 17, and 20, the medium was changed to NB media containing 200 μM dibutyrl cyclic AMP, 200 μM sodium 1-ascorbate, 20 ng/ml NGF, 10 ng/ml BDNF, 10 ng/ml BDNF and 10 ng/ml NT-3. After day 21 of differentiation, half of the spent medium in each well was gently aspirated and supplemented with fresh NB medium containing 200 μM dibutyrl cyclic AMP, 200 μM sodium 1-ascorbate, 20 ng/ml NGF, and 10 ng/ml GDNF. Media was changed every 3 days. Laminin and fibronectin were added to the media every week to maintain attachment.

### FACS Purification of TRPV1+, SCN9A+, and MRGPRX1+ Neurons

One day prior to FACS purification, mature hPSC-derived sensory neurons were fed with fresh NB media containing 200 μM dibutyrl cyclic AMP, 200 μM sodium 1-ascorbate, 20 ng/ml NGF, and 10 ng/ml GDNF. On the day of FACS purification, sensory neuron-conditioned media was harvested from each well, filtered, and supplemented with 10 μM of Y-27632 to make post-sorting media (PSM). hPSC-derived sensory neurons were incubated with TrypLE™ for 30 minutes at 37° C. to dissociate into single cells. Pelleted cells were resuspended in FACS media (90% DPBS+10% DMEM with DNasel, 10 μM of Y-27632, and 3% penicillin-streptomycin) and passed through 50 um strainers. 1 μl Sytox Red (ThermoFisher Scientific, S34859) was added per 1 ml cell suspension. Cells were sorted using SH800S Cell Sorter based on GFP fluorescence and collected in 1.5 ml Eppendorf tubes (filled with 400 ul PSM+3% penicillin-streptomycin). Purified TRPV1+, SCN9A+, or MRGPRX1+ cells were pelleted, resuspended in PSM at a density of 1×10⁴ cells per 5 μl, and replated in 48-well plates pre-coated with poly-lysine/laminin/fibronectin. One 5 μl drop was placed per well. Cells were incubated in 5 μl droplets for 10 minutes at 37° C. to fully attach and added with PSM media. Note that 3% penicillin-streptomycin was added to the FACS media and PSM in the collecting tubes but not to the final replating media.

Purification of CD200+ hPSC-Derived Neurons

One day prior to FACS purification, mature hPSC-derived sensory neurons were fed with fresh NB media containing 200 μM dibutyrl cyclic AMP, 200 μM sodium 1-ascorbate, 20 ng/ml NGF, and 10 ng/ml GDNF. On the day of FACS purification, sensory neuron-conditioned media was harvested from each well, filtered, and supplemented with 10 μM of Y-27632 to make post-sorting media (PSM). hPSC-derived sensory neurons were incubated with TrypLE™ for 30 minutes at 37° C. to dissociate into single cells. Pelleted cells were resuspended in FACS media (90% DPBS+10% DMEM with DNaseI, 10 μM of Y-27632, and 3% penicillin-streptomycin) and passed through 50 um strainers. 1 μl Sytox Red (ThermoFisher Scientific, S34859) was added per 1 ml cell suspension. Mix 10 g of APC/Cy7 Anti-human CD200 antibody *OX-10⁴* (AAT Bioquest, 120001D0) to cells harvested from one plate suspended in 5 mL of FACS media and incubate for 10 min at 37° C.

Resuspend the cells and transfer to sorting tubes (with 50 um strainer cap) using a p1000 micropipette and store the tube on ice. Sort the cells using SH800S Cell Sorter based on GFP fluorescence (for TRPV1+ or SCN9A+ identity), Sytox red (for live cells) and APC/Cy7 fluorescence (for CD200+ identity) and collected in 1.5 ml Eppendorf tubes (filled with 400 μl PSM+3% penicillin-streptomycin). Purified CD200+ cells were pelleted, resuspended in PSM at a density of 1×10⁴ cells per 5 ul, and replated in 48-well plates pre-coated with polylysine/laminin/fibronectin. One 5 ul drop was placed per well. Cells were incubated in 5 ul droplets for 10 minutes at 37° C. to fully attach and added with PSM media. Note that 3% penicillin-streptomycin was added to the FACS media and PSM in the collecting tubes but not to the final replating media.

### Animals and Surgical Procedure

Female and male rats (Charles River, SRO rat, strain code: 707) aged about 50 days were used in the study with approval by the JHU IACUC committee (PI: Dong). Prior to surgery, animals were anesthetized with ketamine (2 mg/lb) and kept under anesthesia for the duration of the surgery.

For transplantation into the DRG, a midline incision was performed through the skin and fascia, and then the surrounding muscles were dissected and retracted laterally to expose the lower lumbar vertebral column. A hemi-laminectomy was performed to expose the spinal cord dura and the attached nerve root and L4 DRG. 0.5 μl cell suspension containing 1×10⁴ TRPV1+ neurons, 2.7×10⁴ SCN9A+ neurons, and 3×10⁴ differentiating neural crest cells were injected into the DRG or the adjacent nerve bundles. Cells were delivered using a glass capillary to minimize physical damage. The glass capillary was left in place for 1 minute to prevent backflow. Grafted animals were maintained and monitored for 4 weeks before left and right L4 DRGs were surgically removed. Left (control) and right L4 DRGs were stained for GFP (Abeam, ab13970) and TUJ1 (ThermoFisher Scientific, MA1-118) to examine the survival of hPSC-derived sensory neurons.

Male NIH III nude from Charles River Laboratories (Wilmington, MA). Mice were anesthetized at 8 weeks of age with xylazine (Rompun, Sedazine, AnaSed; 10 mg/kg, intraperitoneally) and ketamine (Vetalar, Ketaset, Ketalar; 100 mg/kg, intraperitoneally). Then, the anterior cruciate ligament transection (ACLT) surgery was performed to induce instability of the knee. Sham ACLT operations were performed by opening the joint capsule of the knee of independent mice. The tibial tuberosity of sham or ACLT mice was injected with 40 k cells/knee of CD200+ or CD200-hPSC-SNs 2 weeks after surgery.

### Behavioral Testing

Behavioral tests were performed 2 weeks after the cell injection and 4 weeks after surgery. All behavioral tests were performed by the same investigators, who were blinded to the allocation of groups. The von Frey hair was applied perpendicular to the plantar surface of the hind paw (avoiding the toe pads) for 2-3 s. If no response was observed, the next higher strength of hair was used, up to the maximum level of 6 g of bending force. If a withdrawal response occurred, the paw was retested, starting with the next descending von Frey hair until no response occurred. Four more measurements were made after the first difference was observed. The 50% PWT was determined by using the following formula:

50% PWT¼10×fpkd=10,000;
where xf is the exact value (in log units) of the final test of von Frey hair, K is the tabular value for the pattern of the last six positive/negative responses, and d is the mean difference (in log units) between stimuli. The threshold force required to elicit paw withdrawal (median 50% withdrawal) was determined twice on each hind paw (and averaged) on each testing day, with sequential measurements separated by at least 10 min.
   Virus Infection of hPSC-Derived TRPV1+ Neurons

FACS-purified TRPV1+ neurons were cultured for 7 days before infection using AAV PHP.SCAG-tdTomato (MOI=10,000, 20,000, and 100,000). Ad-NULL (MOI=100) was used to coinfect TRPV1+ neurons for improved infection efficiency and tdTomato overexpression. Infected TRPV1+ cells were cultured for additional 5 days, washed for three times in PSM, and injected into animals.

### Immunohistochemical Analysis

Culture neurons were fixed with 4% PFA for 1 hours at room temperature. After 2 washes with PBS, the samples were blocked in PBS with 5% Bovine Serum Albumin (BSA) (Sigma Aldrich) and 0.2% Triton X-100 (Sigma Aldrich) for 1 hour at room temperature and incubated with primary antibodies overnight at 4° C. Primary antibodies were washed 3 times with PBS with 0.3% Triton X-100 (PBST). The samples were incubated with secondary antibodies for 1-2 hours at room temperature. After secondary incubation, slides were washed 3 times with PBST and mounted using VECTASHIELD Antifade Mounting Medium with DAPI (Fisher Vector Lab). The staining was performed using primary antibodies such as BRN3a (Abeam, ab245230), TUJ1 (ThermoFisher Scientific, MA1-118) and appropriate 488, 568, and 647-conjugated Alexa fluor secondary antibodies (Invitrogen) were utilized.

### Single Cell Transcriptomics and Pathway Analysis

R (version 4.3.2) and Seurat (version 5) were used for the single-cell RNA-seq analysis. Sequencing reads were separately acquired for SOX10+, RUNX1+, TRPV1+, SCN9A+ and MRGPRX1+ cells and imported as separate Seurat objects. Data were normalized (NormalizeData) within each group and verified by FeatureScatter (nFeature_RNA vs. nCount_RNA>0.9 for all five groups). Five Seurat objects were then merged into one (merged) with the original identity stored as metadata in each object. ElbowPlot(merged) was performed to calculate the major principle components to avoid over-clustering. To remove technical and other batch-to-batch variations, SCTransform was performed for normalization and variance stabilization of molecular count data from scRNA-seq experiments. Highly similar clusters without clearly distinguishable markers were merged to produce the final 18 clusters. All clusters representing subtypes of mature sensory neurons (i.e., TRPV1+, SCN9A+ and MRGPRX1+) are verified for neuronal markers SNAP25, RBFOX1 and THY1. Standard Seurat analysis pipeline was followed to identify enriched genes in each cluster. Violin plot was used to visualize top genes co-enriched with CD200. Enriched pathways in CD200+ cells were identified using the opensource, open access, manually curated and peer-reviewed Reactome pathway database.

### Statistics

All data are shown as mean±SEM and were subjected to statistical analysis. Significance was analyzed by 1-way ANOVA using Dunnett's or Tukey's multiple-comparisons test or were analyzed by 2-tailed unpaired Student's t test. P≤0.05 was considered significant. The n values indicate the number of independent biological samples. Data were analyzed and represented with GraphPad Prism.

### Example 2. Isolation and Functional Characterization of Molecularly Defined Subsets of Nociceptive and Pruriceptive hPSC-SNs

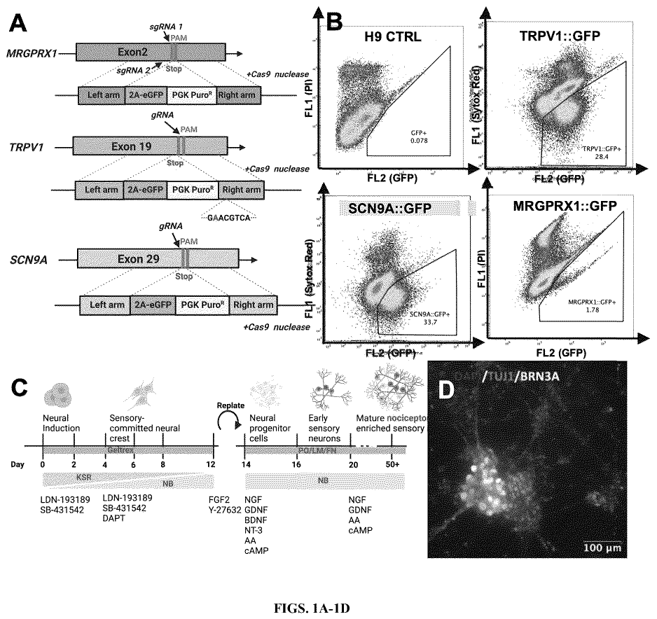

FIGS. 1A-1D illustrate generating hPSC reporter lines. As illustrated, to isolate molecularly defined subsets from hPSC-derived sensory neurons (hPSC-SNs), MRGPRX1::GFP, TRPV1::GFP, and SCN9A::GFP reporter cell lines were generated using the CRISPR-Cas9 system. FIG. 1A illustrate schematic of CRISPR-mediated knock-in construct for MRGPRX1::GFP, TRPV1::GFP, and SCN9A::GFP cell lines. FIG. 1A illustrates a schematic of CRISPR-mediated knock-in construct for MRGPRX1::GFP, TRPV1::GFP, and SCN9A::GFP cell lines. For each locus, the stop codon was replaced with 2A-eGFP-PGK-Puro gene cassette (FIG. 1A). Each clone was separately differentiated into sensory neurons and detected for GFP expression using fluorescence-activated cell sorting (FACS) after 40 DIVs (FIG. 1B) using an optimized protocol (FIG. 1C). The timeline of sensory neuron differentiation protocol is illustrated in FIG. 1C. Representative morphology of hPSC-derived sensory neurons expressing BRN3A (general sensory neuron marker) and TUJI (pan neuronal marker) is illustrated in FIG. 1D. Three clones were confirmed and kept for each line: MRGPRX1::GFP (2A4, 2A8, 2D10), TRPV1::GFP (3B1, 3B4, 3B17), and SCN9A::GFP (3A5, 3A8, 3A19).

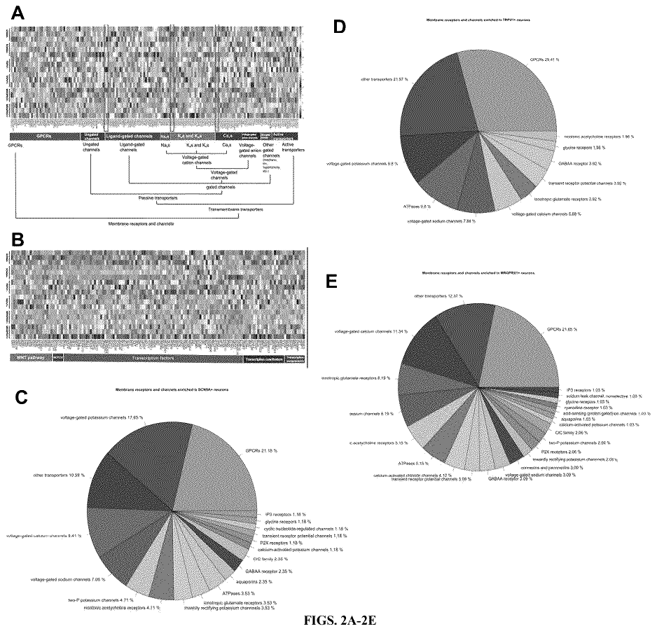

FIGS. 2A-2E illustrate characterizing the transcriptional landscape of MRGPRX1::GFP, SCN9A:: GFP, and TRPV1::GFP sensory neurons. Differentially expressed genes were independently analyzed in each lineage comparison using Gene Set Enrichment Analysis (GSEA) with gene sets from Molecular Signatures Database (MSigDB). To uncover molecular differences among the three subsets of hPSC-SNs, bulk RNA-seq (NextSeq) was performed to identify differentially expressed genes (DEGs) in SCN9A::GFP+ versus SCN9A::GFP−, TRPV1::GFP+ versus TRPV1::GFP−, and MRGPRX1::GFP+ versus MRGPRX1::GFP-hPSC-SNs (70 DIVs). Using gene set enrichment analysis (GSEA), we first analyzed the differential enrichment of membrane receptors and channels (FIG. 2A), as they serve important roles in regulating neuronal excitability and firing rate, making them promising novel therapeutic targets in the treatment of chronic pain. Several voltage-gated potassium channels (e.g., KCNQ2 and KCNG1), active transporters (e.g., SLCJOA4 and ATP2B4), and GPCRs (e.g., ADCYAPIR1) are specifically enriched in TRPV1::GFP+ and SCN9A::GFP+ but not MRGPRX1::GFP+ neurons, suggesting that they play specific roles in the activity or function of nociceptors. On the other hand, some GPCRs (e.g., PRNP, DNM3, UTS2R, HTR2C, HTRJE˜HTR2A, HTRJD, and NPYJR) and voltage-gated calcium channels (e.g., CACNGJ, CACNAJS, CACNG8, and CACNA2D3) are specifically enriched in MRGPRX1::GFP+ but not SCN9A::GFP+ or TRPV1::GFP+ neurons, suggesting for their putative roles in mediating itch. GSEA also reveals differentially regulated pathways and transcription factors in SCN9A::GFP+, TRPV1::GFP+, and MRGPRX1::GFP+ hPSC-SNs (FIG. 2B).

The differential distributions of enriched membrane receptors and channels in SCN9A::GFP+, TRPV1::GFP+, and MRGPRX1::GFP+ hPSC-SNs highlight transcriptomic differences among the three subsets of sensory neurons (FIG. 2C-2E). MRGPRX1::GFP+ hPSC-SNs show the most diverse receptor expression profile, suggesting that they are comprised of a highly homogeneous population of neurons (FIG. 2E).

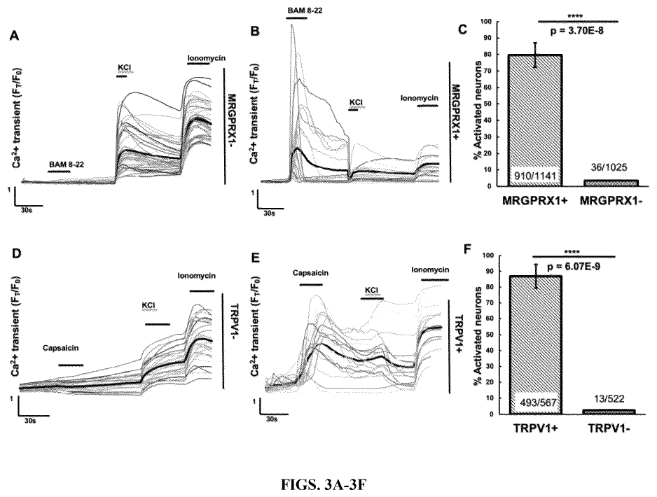

FIGS. 3A-3F illustrate purifiable and excitable populations of hPSC-derived MRGPRX1+, TRPV1+ sensory neurons. To test their functionality, the excitability and purity of sorted MRGPRX1::GFP+, TRPV1::GFP+ cells were examined by their Ca²⁺ transient (Calbryte 590) in response to different pain- and itch-triggering stimuli. MRGPRX1+ neurons displayed significantly heightened response to BAM 8-22 (10 μM) compared to MRGPRX1− neurons (FIG. 3A-3B). Specifically, the purity of sorted MRGPRX1+ and TRPV1+ neurons was tested by quantifying the percentage of activated neurons in response to BAM 8-22 (10 μM) and capsaicin (10 μM) when compared with MRGPRX1− and TRPV1− populations, respectively (FIG. 3A-3F). Specifically, MRGPRX1+ neurons (FIG. 3B) displayed significantly heightened response to BAM 8-22 (10 μM) compared to MRGPRX1− neurons (FIG. 3A) and TRPV1+ neurons (FIG. 3E) displayed significantly heightened response to capsaicin (10 μM) compared to TRPV1-neurons (FIG. 3D).

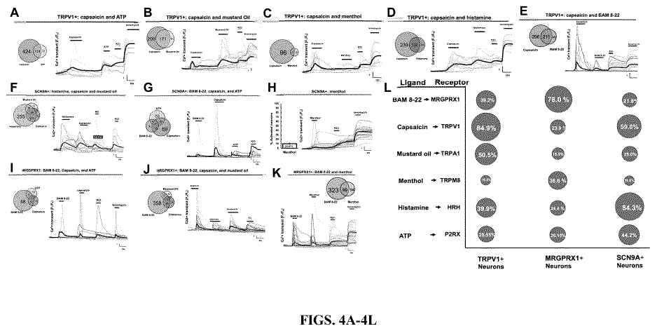

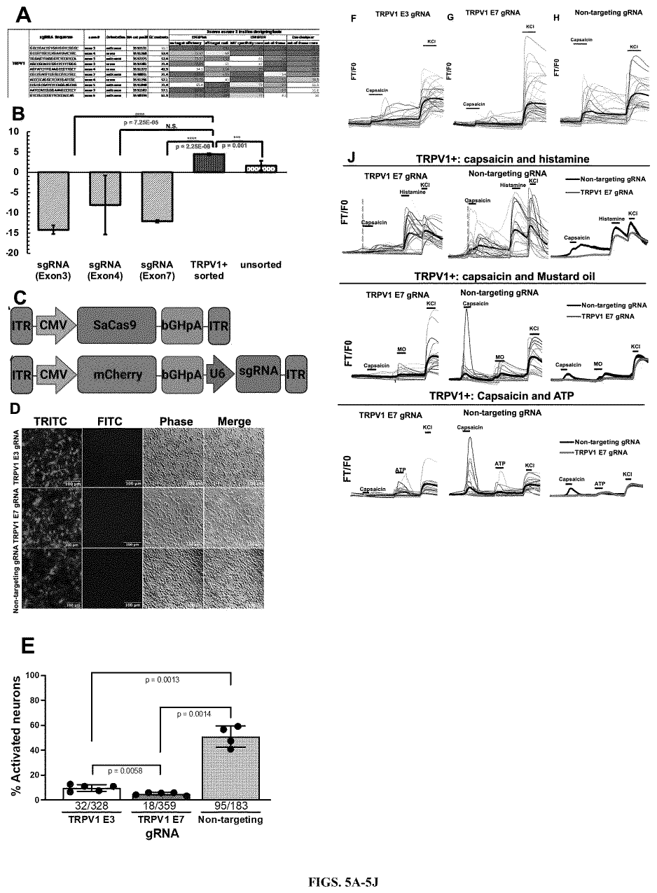

FIGS. 4A-4L. illustrate FACS-purified TRPV1+, SCN9A+, and MRGPRX1+ hPSC-derived sensory neurons responding to various pain- and itch-inducing ligands. The polymodality was examined of TRPV1+ (FIGS. 4A-4E), SCN9A+ (FIGS. 4F-4H), and MRGPRX1+ (FIG. 4I-4K) neurons. The TRPV1+ (FIGS. 4A-4E), SCN9A+ (FIGS. 4F-4H), and MRGPRX1+ (FIG. 4I-4K) neurons were characterized by their response profile to BAM 8-22 (10 μM), capsaicin (10 μM), mustard oil (100 μM), menthol (100 μM), histamine (50 μM), and ATP (50 μM). For example, many of TRPV1+ neurons are responding to mustard oil as well as capsaicin (FIG. 4E) whereas very small numbers of TRPV1+ neurons are responding to menthol (FIG. 4C). In FIG. 4L, we summarized the percentages of each neuron that is responding to each stimulus. Our hPSC-derived MRGPRX1+, TRPV1+, and SCN9A+ neurons displayed distinct response profiles with a preferential response to BAM 8-22, capsaicin, and histamine, respectively, while there is distinctive heterogeneity in terms of their polymodal responsiveness to additional stimuli.

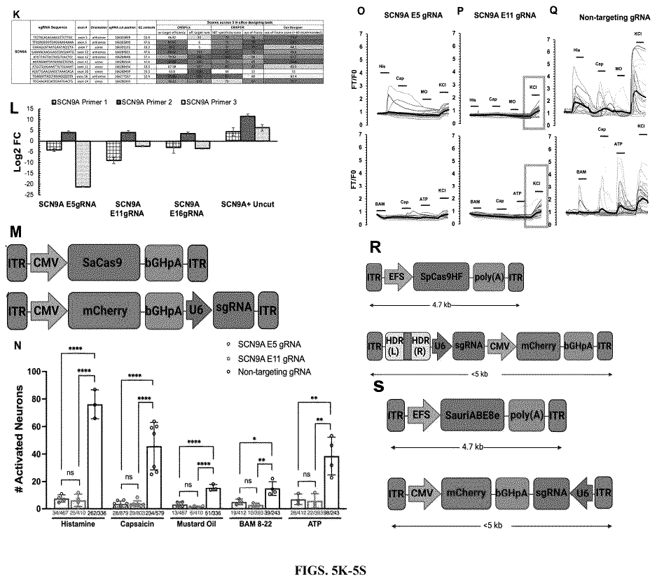

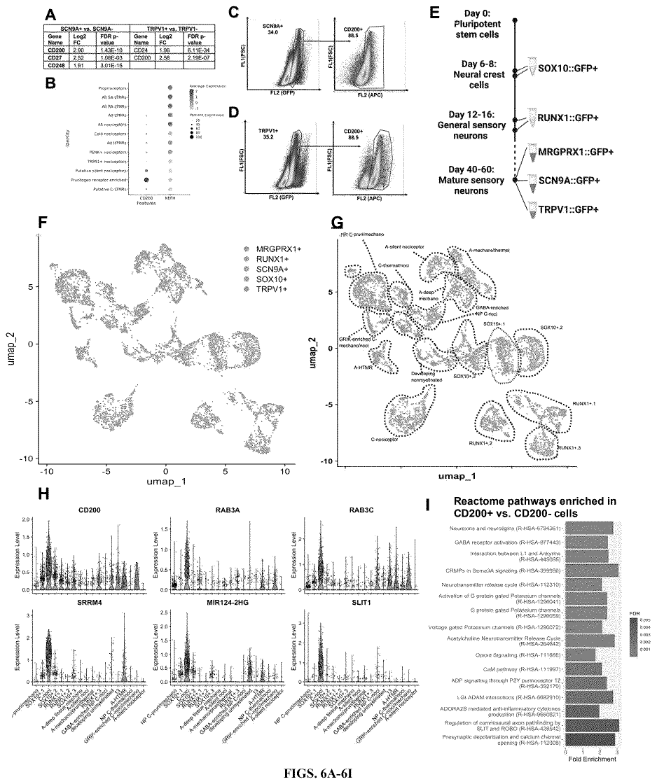

FIGS. 5A-5S. illustrate in vitro optimization and functional validation of candidate CRISPR sgRNAs to enable TRPV1 knockout, and SCN9A knockout. With the goal of developing gene therapy products that provide robust, DRG-specific suppression of chronic pain, we explored the use of CRISPR induced AAV-delivered loss-of-function modulation and silencing (CALMS) to independently target three cation channels in the afferent pain pathway that are vital for the generation of action potentials and the maintenance of resting membrane potential. To establish robust pain silencing in DRG neurons, we first aimed at silencing two major pain targets TRPV1 and SCN9A. We systematically selected 200+ CRISPR sgRNA constructs to enable TRPVJ or SCN9A knockout (FIG. 5A-5Q). Using the most widely used in silica designing tools (CRISPick, CRISPOR. and Cas-Designer), we then selected top 10 candidate sgRNAs targeting early exons of TRPVJ or SCN9A (FIG. 5A, 5K). Three sgRNAs (red) targeting TRPVJ or SCN9A were picked based on three parameters, including high on-target efficiency scores, low off-target effects, and high out-of-frame scores, and were transfected into FACS-purified TRPV1::GFP+ hPSC-SNs (70 DNs) or SCN9A::GFP+ hPSC-SNs (70 DNs) using lipofectamine-mediated delivery.

Knockout efficiencies of TRPV1-knockout sgRNAs (TRPV1-sgRNAs) and SCN9A-knockout sgRNAs (SCN9A-sgRNAs) were compared using qPCR (FIG. SB, SL). The two most efficient TRPV1-sgRNAs and SCN9A-sgRNAs were packaged into AAV PHP.s (a serotype with a strong DRG neuron-tropism) using a dual-AAV delivery strategy (FIG. SC, SM). FACS-purified TRPV1::GFP+ hPSC-SNs (70 DIVs) or SCN9A::GFP+ hPSC-SNs (70 DIVs) were transduced with two TRPV1-sgRNAs and two SCN9A-sgRNAs, respectively (FIG. 5D, data not shown). We then functionally evaluated the efficiencies of AAV-delivered TRPV1-sgRNAs and SCN9A-sgRNAs by examining their Ca²⁺ transients when exposed to capsaicin (10 μM), histamine (50 μM), mustard oil (100 μM), ATP (50 μM), BAM 8-22 (10 μM), and KCl (50 mM). While ES TRP VJ-sgRNA demonstrated specific silencing effects upon the administration of capsaicin (the primary ligand of TRPV1) (FIG. 5F-5J), El 1 SCN9A-sgRNA displayed robust silencing to all five pain-mediating stimuli and KCl (FIG. 5H-5Q). FIG. SR illustrates schematic representation of using CRISPR-mediated HDR delivered by a dual-AAV system to install a protective c.2188A>G (p.Thr730Ala) mutation in KCNQ2. FIG. 5S illustrates schematic representation of using CRISPR-mediated base editing delivered by a dual-AAV system to install a protective c.2188A>G (p.Thr730Ala) mutation in KCNQ2.

FIGS. 6A-6I illustrate single cell RNA-seq data and CD200 as a nociceptor marker. TRKA antibody has been used as the gold standard to isolate nociceptive neurons from heterogenous peripheral neuronal cultures despite the apparent limitation that TRKA is expressed in some non-nociceptive neurons and many nociceptors don't express TRKA (Fang et al., 2005; Saito-Diaz et al., 2021). Discovering a novel non-genetic, antibody-based approach to purify nociceptive neurons from heterogeneous cultures is an important step towards testing the in vitro efficacy of gene therapy strategies in a more generalized context. To this end, we identified top enriched CD markers in TRPV1+ vs. TRPV1−, SCN9A+ vs. SCN9A−, and MRGPRX1+ vs. MRGPRX1− cells, in which CD200 was shown to be significantly enriched in both SCN9A+ and TRPV1+ (but not MRGPRX1+) populations (FIG. 6A). Published human DRG scRNA-seq data also demonstrates that CD200 is specifically enriched in small-fiber primary afferents and clusters differently from NEFH+ large diameter neurons (FIG. 6B, figure generated from raw sequencing data from Tavares-Ferreira et al., 2022). FACS analysis using hPSC-SNs derived from TRPV1::GFP and SCN9A::GFP reporter lines and stained with anti-CD200 (APC/Cy7) acutely before sorting also corroborated the nociceptor-specific enrichment of CD200 (FIG. 6C, 6D). Interestingly, our transcriptomic data showed that the gene counts of CALCA, which encodes a subunit of calcitonin gene-related peptide (CGRP), is enriched in TRPV1+ and SCN9A+ neurons based on the gene counts (Table 1), implicating a therapeutic role of these hPSC-derivednociceptors (marked by CD200 positivity) in promoting tissue healing based on a recent publication (Lu et al., 2024).

TABLE 1
Gene counts of CALCA, CALCB, TRPV1, SCN9A, SOX10 and RUNX1
in mature hPSC-derived TRPV1+ and SCN9A+ sensory
neurons and in early SOX10+ or RUNX1+ sensory neurons.

| | | Gene Name | | | | | |
|---|---|---|---|---|---|---|---|
| | | CALCA | CALCB | TRPV1 | SCN9A | SOX10 | RUNX1 |
| Cell | TRPV1+ | 10536 | 95 | 2600 | 3992 | 107 | 15187 |
| Identity | SCN9A+ | 2472 | 104 | 3296 | 5491 | 109 | 4039 |
| | SOX10+ | 10 | 245 | 3222 | 471 | 2447 | 951 |
| | RUNX1+ | 120 | 7348 | 2452 | 700 | 23504 | 10399 |

The differentiation of hPSC-derived neural crest cells into sensory neuron subtypes emerges as a hierarchical process; for example, Runx1+ nociceptive neuronal precursors branch into peptidergic nociceptors and non-peptidergic nociceptors (Chen et al., 2006). Subsequently, these two populations are sub-diversified by expressing various neuropeptides, receptors, and channels, such as CGRP, RET, Trp channel family, and Mrgpr family (Chen et al., 2006; Kim et al., 2008; Luo et al., 2007). Although most of the specification mechanisms have been studied in mice, the human counterparts remain largely elusive.

To construct the transcriptomic continuum of human peripheral sensory neurons, cells were purified including hPSC-derived SOX10+ neural crest progenitor cells, RUNX1+ sensory progenitor cells as well as TRPV1+, SCN9A+ and MRGPRX1+ sensory neurons during their developmental timing of peak expression in vitro (day 6-8 for neural crest progenitor cells, day 12-16 for early sensory neuron progenitor cells, and day 50-70 for mature sensory neurons) (FIG. 6E). Sequencing libraries were generated following the Smart-seq2 protocol (Ramskold et al., 2012). A total of >30,000 cells passed through the final quality control for sequencing. 1,000 cells randomly sampled from each of the five groups were merged and clustered based on their transcriptomic identities for further analysis (FIG. 6F). Eighteen transcriptomic clusters of developing and mature hPSC-derived DRG neurons were identified using Seurat and published human DRG neuron subtype markers (Hao et al., 2021; Lause et al., 2021; Nguyen et al., 2021; Tavares-Ferreira et al., 2022; Yu et al., 2023). Six clusters representing sensory neuron precursor cells were assigned the identities “SOX10+” or “RUNX1+”. Twelve sensory neuron subtypes were assigned, among which five are C-fiber nociceptors, four are A-fiber mechano nociceptors, two are of unknown identities, and one cluster represents a developing population of unmyelinated lineage (FIG. 6G). CD200 is enriched in clusters “C-nociceptors”, “developing unmyelinated lineage”, “cluster 13”, “A-HTMR” and “GRIK-enriched Cnoci/mechanoreceptors”, along with some low expression in SOX10+ and RUNX1+ sensory neuron precursor cells (FIG. 6H). Interestingly, RAB3A and RAB3C, two genes shown to promote the secretion of neuronal cell-derived extracellular (EV) vesicles during repetitive stimulation (Yang et al., 2024), show high co-enrichment patterns with CD200 (FIG. 6H).

Moreover, MiR-124, a miRNA shown to inhibit inflammatory responses through polarizing macrophages to favor the M2 phenotype (Essandoh et al., 2016; Qin et al., 2016), is also coenriched with CD200. These pieces of evidence indicate an attractive potential of CD200+ neurons to exhibit heightened ability of secreting EV s during continuing stimulation and secrete anti-inflammatory factors such as miRNAs to polarize the phenotype of local macrophages. To investigate relevant pathways enriched in CD200+ neurons, we performed Reactome pathway analysis and identified signaling pathways that modulate the nociceptive pathways (e.g., GABA receptor activation, neurotransmitter release cycle, and opioid signaling) or local inflammatory environment (e.g., ADORA2B mediated anti-inflammatory cytokines production) are among the top enriched pathways of CD200+ compared to CD200− neurons (FIG. 6I). Collectively, these data show that CD200 can serve as a reliable, specific nociceptor marker to purify cultures of primary sensory afferents in the absence of any genetic reporter system and exhibit attractive therapeutic potentials when applied in a cell therapy context.

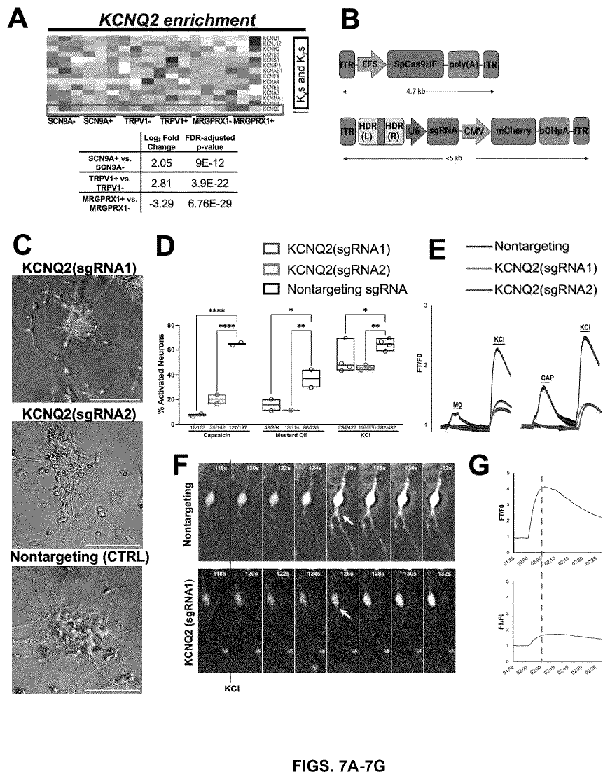

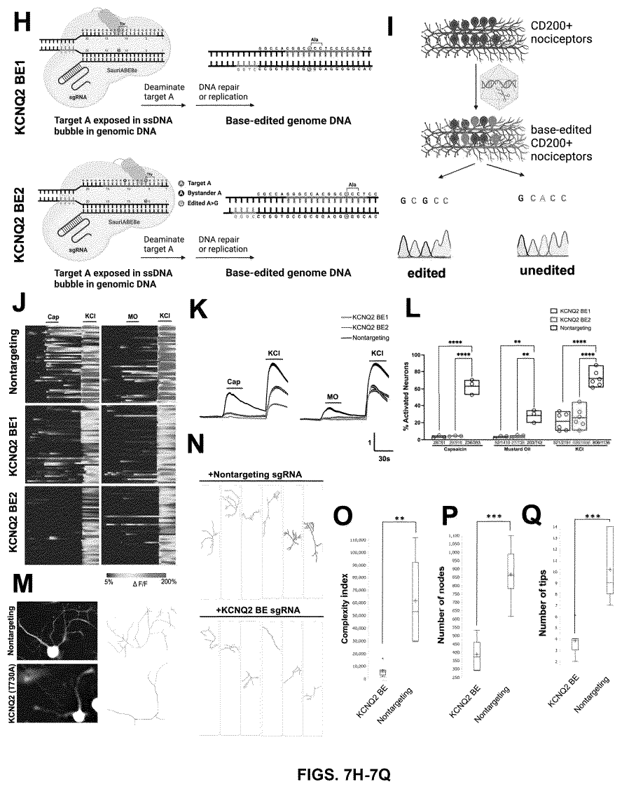

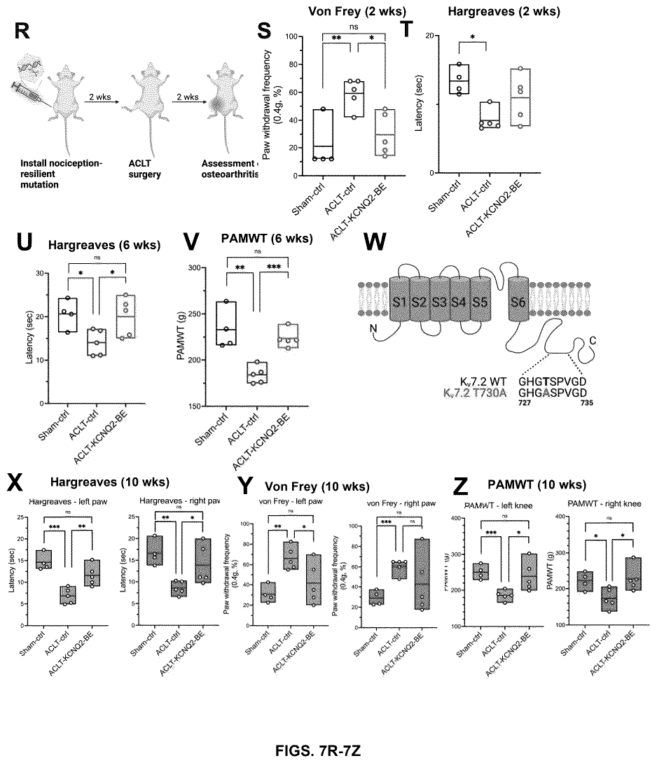

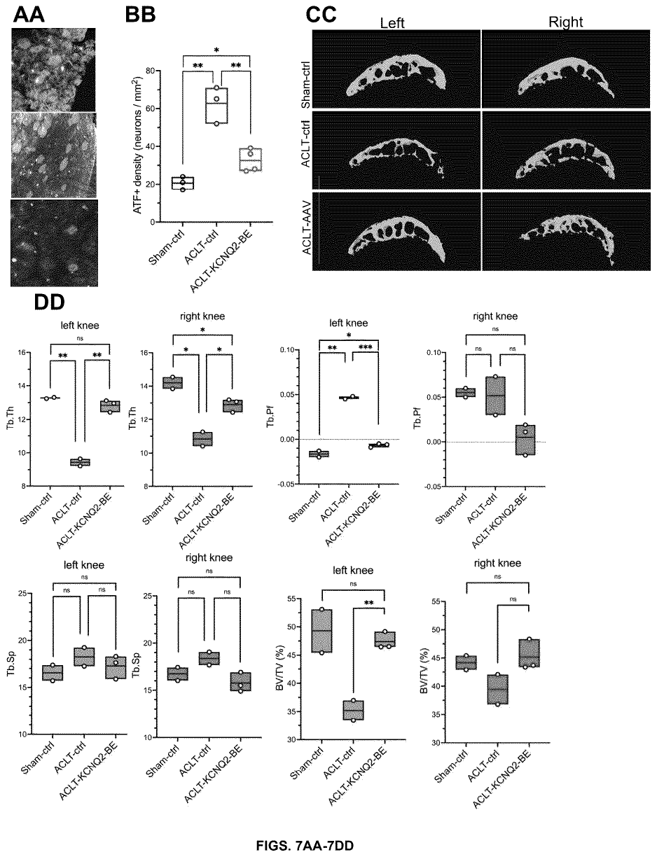

FIGS. 7 A-7DD. illustrate a KCNQ2 as a gene therapy target gene conferring robust pain-resilience effects in hPSC-derived sensory neurons and the KCNQ2 gene therapy efficacy in a rodent model resulting in bone remodeling. To identify such novel targets, the molecular profiles of TRPV1+, SCN9A+, and MRGPRX1+ neurons was analyzed. Given the vital roles of G-protein-coupled receptors (GPCRs) and ion channels in transducing noxious stimuli, generating and propagating action potentials and maintaining resting membrane potentials, DEG analysis was performed and GSEA on SCN9A+ vs. SCN9A1−, TRPV1+ vs. TRPV1−, and MRGPRX1+ vs. MRGPRX1− neurons using membrane receptor-related gene sets to identify novel pain targets. This assessment identified potassium voltage-gated channel subfamily Q member 2 (KCNQ2) is significantly enriched both in TRPV1::GFP+ neurons (Log 2 FC=2.81, p-value=3.91E-22) and SCN9A::GFP+ neurons (Log 2 FC=2.05, p-va]ue=9.00E-12), but downregulated in MRGPRX1::GFP+ neurons (Log 2 FC=−3.29, p-value=6.76E-29), suggesting that its role in maintaining resting membrane potential and regulating neuronal excitability is specific to nociceptive neurons (FIG. 7A).

In a separate study, Waxman's group identified a gain-of-function mutation in KCNQ2 (p.T730A or c.2188A>G) that causes a hyperpolarizing shift in resting membrane potential by −5 mV, which conferred considerable resilience to pain in some patients with IEM, a well characterized human genetic model of chronic pain (Mis et al., 2019). Pharmacological modulators for KCNQ2 (retigabine and flupirtine) had been used in clinics but were later withdrawn due to significant side effects. Efforts to develop pharmacological KCNQ2 modulators have been limited, partly due to its expression in the Central Nervous System (CNS).

In an alternative approach, by installing KCNQ2 (p.T730A) mutation in mature CD200-purified hPSC-SNs using CRISPR-Cas-mediated HDR, in which SpCas9 expressed under a compact promoter elongation factor 1α short (EFS) and a HDR template carrying the desired edit and a sgRNA targeting KCNQ2 tagged by mCherry are separately packaged into AAV-PHP.s. SpCas9 was selected over other Cas9 variants due to its broad PAM compatibility (3′NGG), which enabled the selection of two sgRNAs that cut 1 bp and 6 bp away from the mutation site, respectively (FIG. 7B, see Table 2 for a complete list of sgRNAs designed to enable the p.T730A single-base editing of KCNQ2). To improve editing efficiency, we tested a broad range of multiplicity of infection (MOI) and different ratios of AAV-SpCas9/AAV-HDR-sgRNA and found out that AAV-SpCas9/AAV-HDR-sgRNA administered at MOI=20,000 and MOI=40,000, respectively, yielded the best infection efficiency with >70% CD200-purified neurons labeled by mCherry (FIG. 7C). To functionally evaluate pain-resilient effects of KCNQ2 (p.T730A), we examined the Ca2+ transients of edited CD200-purified neurons when exposed to capsaicin, mustard oil, and KCl. Results demonstrate that the KCNQ2 (p.T730A) mutation significantly dampened the response magnitude and decreased the percentage of activated neurons (FIG. 7F-7G). Notably, neurons installed with KCNQ2 (p.T730A) mutation displayed protracted response to KCl and significantly diminished Ca2+ influx at axonal initial segment and distal axon (FIG. 7F).

Exemplary protospacer sequences of the sgRNA are described in Table 2 below.

TABLE 2
CRISPR-mediated HDR and CRISPR-mediated base editing designs to install the
protective KCNQ2 (c.2188A > G, p.Thr730Ala) mutation in primary sensory neurons as a
treatment of chronic pain.
NM_172107.4(KCN02): c.2188A > G (p.Thr730Ala)

| | SpCas9 sgRNA | | | Cut to | |
|---|---|---|---|---|---|
| | Protospacers (5′-3′) | Strand | PAM | [symbol] | HDR |
| CRISPR- | CCCCCACGGGGGA | − | TGG | 1 | +Strand |
| mediated | GGTGCCG | | | | CCTCCTGGCAGCCACAGAGC |
| HDR | | | | | CACCCGCGCCAGGGCCACG |
| | (SEQ ID NO: 32) | | | | GCGCCTCCCCCGTGGGGGAC |
| | | | | | CACGGCTCCCTGGTGCGCAT |
| | GCCGTGGTCCCCCA | − | AGG | 6 | +Strand |
| | CGGGGG | | | | CTCCTGGCAGCCACAGAGCCACCC |
| | (SEQ ID NO: 35) | | | | GCGCCAGGGCCACGGCGCCTCCCC |
| | | | | | CGTGGGGGACCACGGCTCCCTGGT |
| | | | | | GCGCATCCCGCCGCC (SEQ ID NO: |
| | | | | | 36) |
| | GGCCACGGCACCT | + | GGG | 7 | +Stand |
| | CCCCCGT | | | | CTCCTGGCAGCCACAGAGCCACCC |
| | (SEQ ID NO: 38) | | | | GCGCCAGGGCCACGGCGCCTCCCC |
| | | | | | CGTGGGGGACCACGGCTCCCTGGT |
| | | | | | GCGCATCCCGCCGCCG (SEQ ID NO: |
| | GGGCCACGGCACC | + | TGG | 6 | +Strand |
| | TCCCCCG | | | | CTCCTGGCAGCCACAGAGCCA |
| | (SEQ ID NO: 41) | | | | CCCGCGCCAGGGCCACGGCGC |
| | | | | | CTCCCCCGTGGGGGACCACGG |
| | | | | | CTCCCTGGTGCGCATCCCGCCG |
| | | | | | CC (SEQ ID NO: 42) |
| | GCCACGGCACCTCC | + | GGG | 8 | +Strand |
| | CCCGTG | | | | CTCCTGGCAGCCACAGAGCCACCC |
| | (SEQ ID NO: 44) | | | | GCGCCAGGGCCACGGCGCCTCCCC |
| | | | | | CGTGGGGGACCACGGCTCCCTGGT |
| | | | | | GCGCATCCCGCCGCCGC (SEQ ID |
| | | | | | NO. 45) |
| | CGGGGGAGGTGCC | − | TGG | 7 | +Strand |
| | GTGGCCC | | | | CCTCCACCTCCTGGCAGCCACA |
| | (SEQ ID NO: 47) | | | | GAGCCACCCGCGCCAGGGCCA |
| | | | | | CGGCGCCTCCCCCGTGGGGGAC |
| | | | | | CACGGCTCCCTGGTGCGCATCC |
| | | | | | [symbol] (SEQ ID NO: 48) |
| | AGCCACCCGCGCC | + | CGG | 7 | +Strand |
| | AGGGCCA | | | | CCTCCACCTCCTGGCAGCCACA |
| | (SEQ ID NO: 50) | | | | GAGCCACCCGCGCCAGGGCCA |
| | | | | | CGGCGCCTCCCCCGTGGGGGAC |
| | | | | | CACGGCTCCCTGGTGCGCATCC |
| | | | | | C (SEQ ID NO: 51) |
| | GGAGCCGTGGTCC | − | GGG | 9 | +Strand |
| | CCCACGG | | | | CTCCTGGCAGCCACAGAGCCACCC |
| | (SEQ ID NO: 53) | | | | GCGCCAGGGCCACGGCGCCTCCCC |
| | | | | | CGTGGGGGACCACGGCTCCCTGGT |
| | | | | | GCGCATCCCGCCGCCGCC (SEQ ID |
| | | | | | NO: 54) |
| | | | | | −Strand |
| | | | | | GGCGGCGGCGGGATGCGCACCAG |
| | CCACGGCACCTCCC | + | GGG | 9 | +Strand |
| | CCGTGG | | | | CTCCTGGCAGCCACAGAGCCACCC |
| | (SEQ ID NO: 56) | | | | GCGCCAGGGCCACGGCGCCTCCCC |
| | | | | | CGTGGGGGACCACGGCTCCCTGGT |
| | | | | | GCGCATCCCGCCGCCGCC (SEQ ID |
| | | | | Cut | |
| | SauriCas9 sgRNA | Strand | PAM | to | Base editor |
| CRISPR- | TCCCCCACGGGGG | − | GTG | 1 | ABE8e |
| mediated | AGGTGCCG (SEQ ID | | G | | |
| base | NO: 59) | | | | |
| editing | AGCCGTGGTCCCCC | − | GA | 6 | |
| | ACGGGGG (SEQ ID | | GG | | |
| | NO: 60) | | | | |
| | GGGCCACGGCACC | + | TGG | 7 | |
| | TCCCCCGT (SEQ ID | | G | | |
| | NO: 61) | | | | |
| | AGGGCCACGGCAC | + | GTG | 6 | |
| | CTCCCCCG (SEQ ID | | G | | |
| | NO: 62) | | | | |
| | GGCCACGGCACCT | + | GG | 8 | |
| | CCCCCGTG (SEQ ID | | GG | | |
| | NO: 63) | | | | |
| | ACGGGGGAGGTGC | − | CTG | 7 | |
| | CGTGGCCC (SEQ ID | | G | | |
| | NO: 64) | | | | |
| | GAGCCACCCGCGC | + | AC | 7 | |
| | CAGGGCCA (SEQ ID | | GG | | |
| | NO: 65) | | | | |
| | GGGAGCCGTGGTC | − | GG | 9 | |
| | CCCCACGG (SEQ ID | | GG | | |
| | NO: 66) | | | | |
| | GCCACGGCACCTCC | + | GG | 9 | |
| | CCCGTGG (SEQ ID | | GG | | |
| | NO: 67) | | | | |
| | CAGGGAGCCGTGG | − | CG | 11 | |
| | TCCCCCAC (SEQ ID | | GG | | |
| | NO: 68) | | | | |
| | GCCACAGAGCCAC | + | AG | 13 | |
| | CCGCGCCA (SEQ ID | | GG | | |
| | NO: 69) | | | | |
| | CCAGGGAGCCGTG | − | AC | 12 | |
| | GTCCCCCA (SEQ ID | | GG | | |
| | NO: 70) | | | | |
| | GGAGGTGCCGTGG | − | GC | 12 | |
| | CCCTGGCG (SEQ ID | | GG | | |
| | NO: 71) | | | | |
| | AGCCACAGAGCCA | + | CA | 14 | |
| | CCCGCGCC (SEQ ID | | GG | | |
| | NO: 72) | | | | |
| | GAGGTGCCGTGGC | − | CG | 13 | |
| | CCTGGCGC (SEQ ID | | GG | | |
| | NO: 73) | | | | |
| | CACCTCCCCCGTGG | + | AC | 16 | |
| | GGGACCA (SEQ ID | | GG | | |
| | NO: 74) | | | | |
| | GTGCCGTGGCCCTG | − | GTG | 16 | |
| | GCGCGGG (SEQ ID | | G | | |
| | NO: 75) | | | | |
| | GGGATGCGCACCA | − | GTG | 22 | |
| | GGGAGCCG (SEQ ID | | G | | |
| | NO: 76) | | | | |
| | CCGTGGGGGACCA | + | CTG | 24 | |
| | CGGCTCCC (SEQ ID | | G | | |
| | NO: 77) | | | | |
| | SpCas9 sgRNA | — | M | — | Base editor |
| KCNQ2 | GGCCACGGCACCT | − | PAGG | — | — |
| hBE1-4 | CCCCCGTG | | GG | | |
| | (SEQ ID NO: 78) | | | | |
| | CAGGGCCACGGCA | − | GTG | — | — |
| | CCTCCCCC | | G | | |
| | (SEQ ID NO: 79) | | | | |
| | AGGGCCACGGCAC | − | TGG | — | — |
| | CTCCCCCG | | G | | |
| | (SEQ ID NO: 80) | | | | |
| | GGGCCACGGCACC | − | GG | — | — |
| | TCCCCCGT | | GG | | |
| | (SEQ ID NO: 81) | | | | |

[symbol]

indicates data missing or illegible when filed

Due to the generation of DNA double strand break (DSB), CRISPR HDR-based editing has been reported to introduce unwanted indels at the locus of editing (Chiruvella et al., 2013; Lieber, 2010). To improve the translational potential of KCNQ2 (p.T730A) editing, we designed two CRISPR BE-based strategies, in which SauriCas9-ABE8e, a DNA deaminase fused with D10A nCas9, catalyzes the deamination of adenosine to guanine without the need to generate D SB. Since the editing window of SauriCas9-ABE8e typically ranges from protospacer positions 3-16 (counting the PAM as positions 22-25), with optimal editing occurring between positions 5-15 (Anzalone et al., 2020), we designed the two sgRNAs to target the complementary strand so that the KCNQ2 (c.2188A>G) of the desired strand is placed at protospacer positions 12 and 6, respectively. While KCNQ2 BE1 installs the desired ACC (threonine) to GCC (alanine) edit with no bystander mutations, KCNQ2 BE2 also installs a CAC (histidine) to CGC (arginine) bystander mutation due to the presence of two A's in the editing window (FIG. 7H).

We next performed TA cloning and Sanger sequencing to verify the successful installation of the mutation and estimate the editing efficiency. Results demonstrated that 16 out of 21 clones harbored edited KCNQ2 (c.2188A>G) mutation, yielding an estimated efficiency of 76% in CD200 purified hPSC-SNs (FIG. 7I). To functionally examine the nociception-resilient effects of KCNQ2 (c.2188A>G) installed by CRISPR BE-mediated editing, we examined the Ca²⁺ transients of edited CD200-purified neurons when exposed to capsaicin, mustard oil, and KCl. Results demonstrated that the KCNQ2 (c.2188A>G) mutation significantly dampened the response magnitude and decreased the percentage of activated neurons (FIG. 7J-7L). Notably, morphological reconstruction and Simple Neurite Tracer (SNT) analysis (Arshadi et al, 2021) demonstrated that edited neurons showed fewer neuronal processes invaded by Ca2+ upon KCl administration (FIG. 7M-7N), evidenced by the diminished complexity index, number of nodes and number of tips (FIG. 70-7Q) compared to the unedited neurons.

We next examined the efficacy of KCNQ2 (c.2188A>G) mutation in alleviating osteoarthritic pain in a mouse model of osteoarthritis (OA). We performed anterior cruciate ligament transection (ACLT) surgery to induce OA in male and female wildtype mice preinstalled with the KCNQ2 (c.2188A>G) mutation via direct injection into the knee two weeks prior to the surgery and assessed for pain profiles two weeks after the surgery (FIG. 7R). Von Frey test showed a significant improvement in mechanical hypersensitivity in animals installed with KCNQ2 (c.2188A>G) 2 weeks after the surgery (FIG. 7S). Hargreaves test of the paw showed a favorable trend in the improvement of thermal hypersensitivity in OA mice 2 weeks after the surgery (FIG. 7T).

Hargreaves test of the paw repeated 6 weeks after surgery showed a significant improvement of thermal hypersensitivity in OA mice (FIG. 7U). Primary knee hyperalgesia was analyzed 6 weeks after the surgery by measuring withdrawal threshold during direct knee press using a pressure application measurement (PAM) force transducer. PAM withdrawal threshold (PAMWT) of animals installed with KCNQ2 (c.2188A>G) showed significant improvement in primary knee hyperalgesia (FIG. 7V). To investigate whether the direct left knee injection of AAV could confer long-lasting analgesic effects and show efficacy at the contralateral knee, we monitored animals for 10 weeks after the ACLT surgery. Hargreaves and Von Frey test at bilateral paws showed significant improvements of OA-induced thermal and mechanical hypersensitivity in QA animals receiving the AAV, to a level comparable to that of animals receiving the sham surgery (FIG. 7X-7Y).

PAMWT demonstrated significant improvements of bilateral primary knee hyperalgesia in animals receiving direct injection of KCNQ2 (c.2188A>G) at the left knee, although the right knee (contralateral to injection) showed less improvements compared to the left knee (FIG. 7Z). To validate the bilateral long-term analgesic effects conferred by the KCNQ2 (c.2188A>G) installation at a biomolecular level, bilateral DRGs were harvested that innervate the knee joints (L3-L4 DR Gs) at the 10-week endpoint. The expression of ATF3 (a neuronal injury marker) was elevated in DRGs from OA mice that received the control AAV (ACLT-Ctrl), but the heightened ATF3 expression was revered in DRGs of OA mice that received the KCNQ2 (c.2188A>G) AA V (ACLT-AA V), to a level comparable to animals that received the sham surgery (sham-Ctrl) (FIGS. 7AA-7BB). OA-associated bone loss is one of many pathological changes seen in joint tissues of individuals (El-Sherif et al., 2008, Haara et al., 2005, Simon et al., 2020). To examine whether the installation of KCNQ2 (c.2188A>G) could improve OA-induced bone loss, we performed microCT scans of bilateral knee joints harvested at 10 weeks after the ACLT surgery (FIG. 7CC). Animals receiving KCNQ2 (c.2188A>G) showed reversed QA-induced bone loss at left and right knees 10 weeks after the ACLT surgery to a level comparable to the sham-operated group, evidenced by improved trabecular thickness (Tb.Th), trabecular bone pattern factor (Tb.Pf), trabecular separation (Tb.Sp) and trabecular bone volume (BV/TV), although the reversal of bone loss seen in the right knee was to a lesser extent than the left knee (FIG. 7DD).

Altogether, these findings have identified KCNQ2 as a novel pain target, designed and optimized two genetic strategies to install a protective mutation KCNQ2 (c.2188A>G) in hPSC-SN s via 1) a CRISPR-mediated HDR template approach and 2) a CRISPR-mediated base editing approach, and validated the long-lasting analgesic effects of the optimized KCNQ2 editing construct in a mouse model of QA. In addition to the bilateral long-lasting pain-resilient effects, we also observed that our gene therapy strategy provided a robust reversal of bone loss in bilateral knees in OA animals.

OA-associated bone loss is one of many pathological changes seen in joint tissues of individuals (El-Sherif et al., 2008, Haara et al., 2005, Simon et al., 2020). To examine whether the installation of KCNQ2 (c.2188A>G) could improve OA-induced bone loss, we performed microCT scans of bilateral knee joints harvested at 10 weeks after the ACLT surgery (FIG. 7 CC). Animals receiving KCNQ2 (c.2188A>G) showed reversed OA-induced bone loss at left and right knees 10 weeks after the ACLT surgery to a level comparable to the sham-operated group, evidenced by improved trabecular thickness (Tb.Th), trabecular bone pattern factor (Tb.Pf), trabecular separation (Tb.Sp) and trabecular bone volume (BV/TV), although the reversal of bone loss seen in the right knee was to a lesser extent than the left knee (FIG. 7DD). Altogether, we have identified KCNQ2 as a novel pain target, designed and optimized two genetic strategies to install a protective mutation KCNQ2 (c.2188A>G) in hPSC-SN s via 1) a CRISPR-mediated HDR template approach and 2) a CRISPR-mediated base editing approach, and validated the long-lasting analgesic effects of the optimized KCNQ2 editing construct in a mouse model of OA. In addition to the bilateral long-lasting pain-resilient effects, we also observed that our gene therapy strategy provided a robust reversal of bone loss in bilateral knees in OA animals.

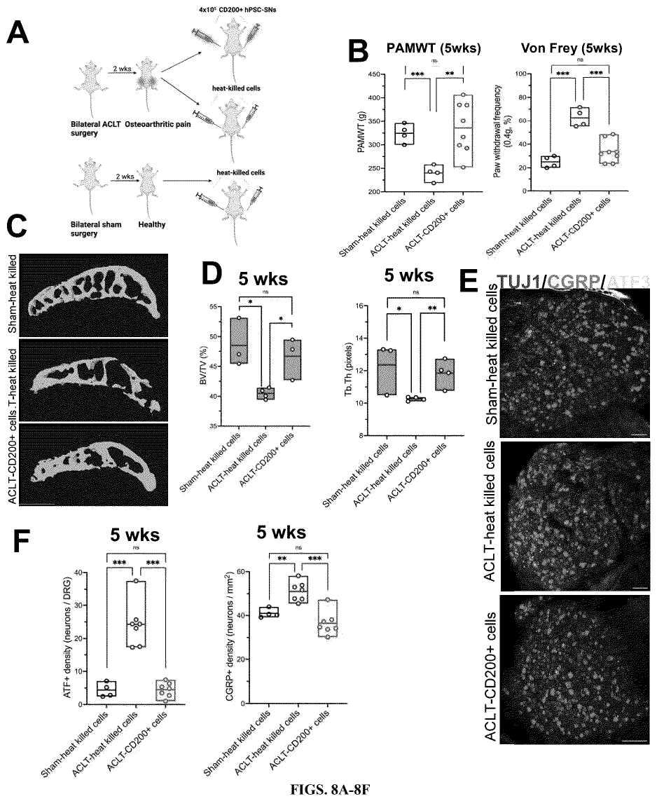

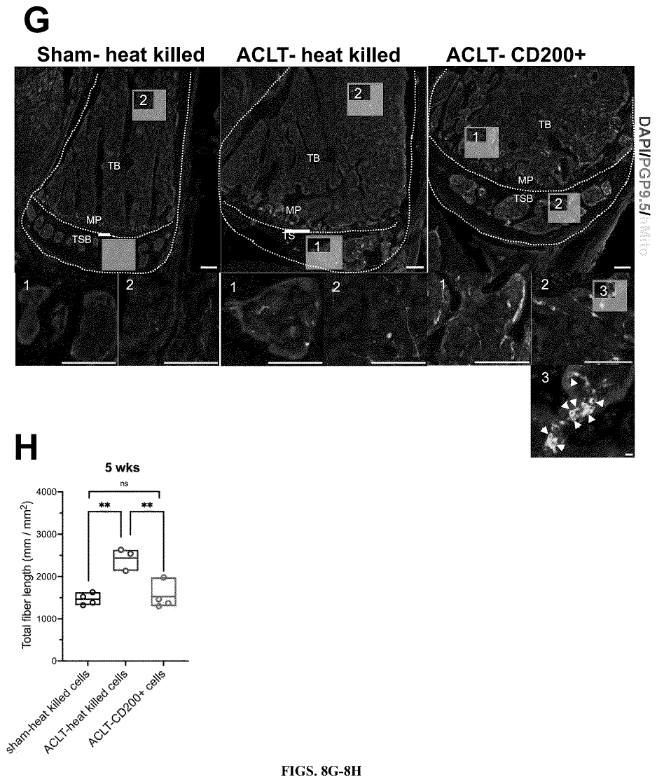

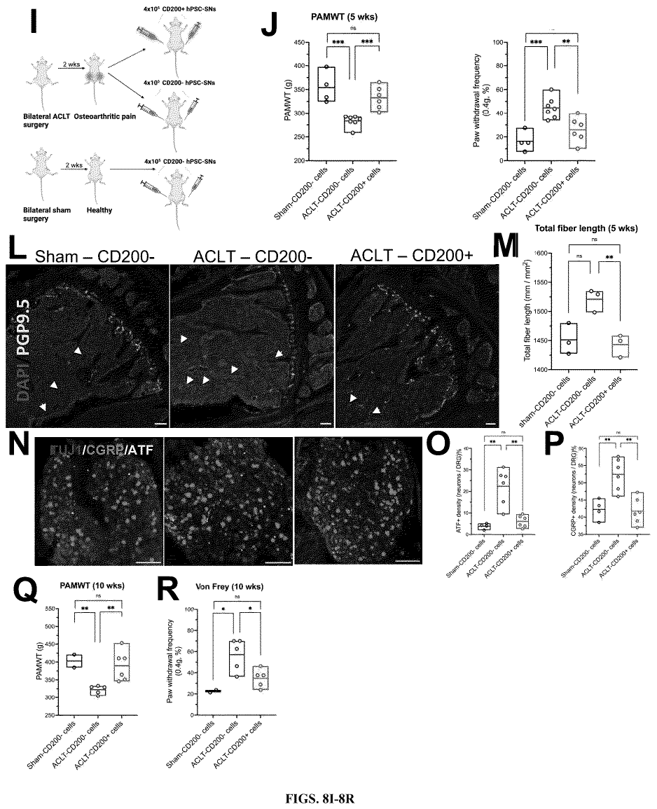

FIGS. 8A-8R illustrate CD200+ sensory neuron injection to intercept pain to treat localized pain and improve the bone remodeling by injecting hPSC-derived pain-sensing neurons. As purified CD200+ hPSC-SNs are enriched for genes conferring anti-inflammatory potentials to polarize local infiltrating macrophages and signaling pathways that modulate the nociceptive pathway, they present excellent opportunities for cell therapy to treat chronic pain caused by inflammation. We next explored the use of purified CD200+ hPSC-SNs in treating a form of localized inflammatory pain in OA. We injected CD200+ hPSC-SN s into the tibial tuberosity of immunocompromised NIH III nude mice after bilateral anterior cruciate ligament transection (ACLT) surgery-induced QA pain (FIG. 8A). We observed that animals receiving CD200+ cell injection displayed a mitigated mechanical pain hypersensitivity profile compared to the OA animals receiving heat-killed cells five weeks after injection (FIG. 8B). We also observed that OA-induced bone loss in the knee joint was reversed in the treated group at the five-week timepoint (FIG. 8C-8D). To validate the analgesic effects conferred by CD200+ injection at a biomolecular level, we isolated bilateral L3-IA DRGS (the DRGs that innervate knee joints in mice) to assess the expression of cell injury and pain markers (i.e., ATF3 and CGRP) and found that animals that underwent the ACLT surgery but received heat-killed cells showed heightened expression of both ATF3 and CGRP. However, the expression of ATF3 and CGRP were decreased in ACLT-animals that received CD200+ cells, to a level indistinguishable from animals that underwent sham surgery (FIGS. 8E-8F).

A substantial body of evidence indicates that OA involves additional recruitment of nerves that innervate the subchondral bone, and that this increased innervation is tightly associated with pain behavior in animals (Aso et al., 2020; Morgan et al., 2022). To examine whether the improved pain behavior in OA animals receiving CD200+ cells is associated with reversed recruitment of nerves innervating the knee joint, bilateral knee joints were harvested from animals at the five-week timepoint to quantify total fiber lengths (FIG. 8G). NeuroJ analysis using fluorescent intensity of PGP9.5 that stained for endogenous nerves demonstrated that the subchondral bone zone and tibia of ACLT-CD200+ animals showed significantly less innervation compared to ACLT-heat killed animals (FIG. 8H). Moreover, human-specific mitochondrial antibody (hMito) was showed that injected CD200+ neurons were localized in the tibial bone and subchondral bone area (FIG. 8G). To exclude the alternative hypothesis that a general population of hPSC-SNs could also confer analgesic and bone uncoupling potentials, we next injected CD200+ hPSC-SNs into the tibial tuberosity of immunocompromised NIH III nude mice after bilateral ACLT, whereas the control animals were injected with CD200− cells (FIG. 8I).

At the five-week timepoint, PAMWT and Von Frey results demonstrated that ACLT-CD200+ animals showed significantly improved mechanical hypersensitivity while ACLT-CD200− animals maintained OA-induced mechanical hypersensitivity with no significant improvements (FIG. SJ-SK). To examine whether the improved pain behavior in OA animals receiving CD200+ cells is associated with reversed recruitment of nerves innervating the knee joint, we harvested bilateral knees at the five-week timepoint to quantify total fiber lengths (FIG. SL). NeuroJ analysis using fluorescent intensity of PGP9.5 that stained for endogenous nerves demonstrated that the subchondral bone zone and tibia of ACLT-CD200+ animals showed significantly less innervation compared to ACLT-CD200− animals (FIG. 8M). To assess the analgesic effects of CD200+ neurons over CD200− neurons in the context of OA at a biomolecular level, we harvested bilateral L3-L4 DRGs innervating knee joints and quantified the expression level of ATF3 and CGRP. Results demonstrated that DRGs from ACLT-CD200− mice expressed significantly higher levels of ATF3 and CGRP 5 weeks after the injection, an elevation reversed by CD200+ neuron injection (FIGS. 8O-8P). To assess the long-term efficacy of CD200+ neurons injection, we examine mechanical hypersensitivity of animals 10 weeks after injection. Von Frey and direct pressure at knee tests demonstrated that ACLT animals receiving CD200+ neurons displayed significantly improved mechanical hypersensitivity compared to ACLT animals receiving CD200− neurons, to a level indistinguishable from the control (FIGS. 8Q-8R). Altogether, we have demonstrated that the SNIIP strategy using purified CD200+ hPSC-SNs has potential to not only alleviate OA-induced pain, but also modulates the pathological bone coupling in OA and reversed OA-induced bone loss. We anticipate the use of CD200+ hPSC-SNs is not limited to OA and have great potentials for treating other pathological pain conditions.

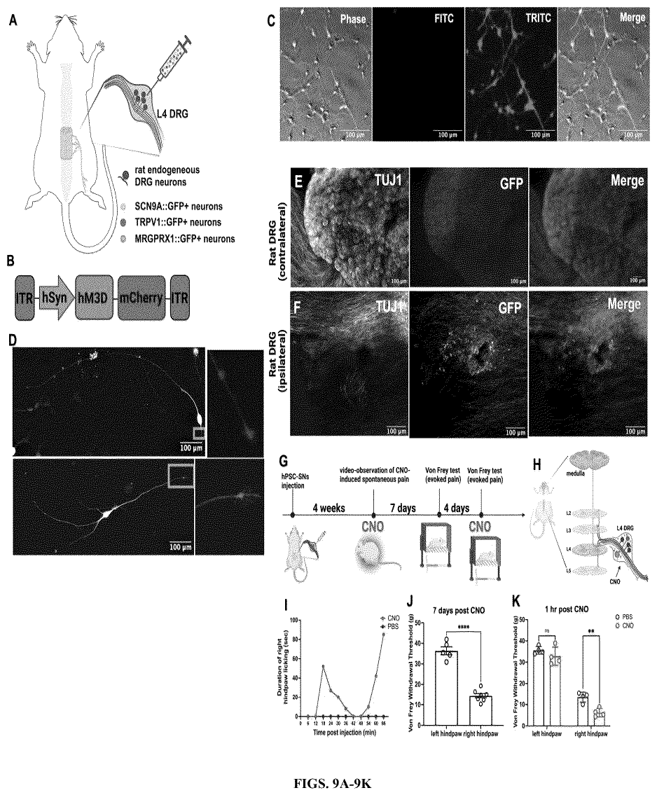

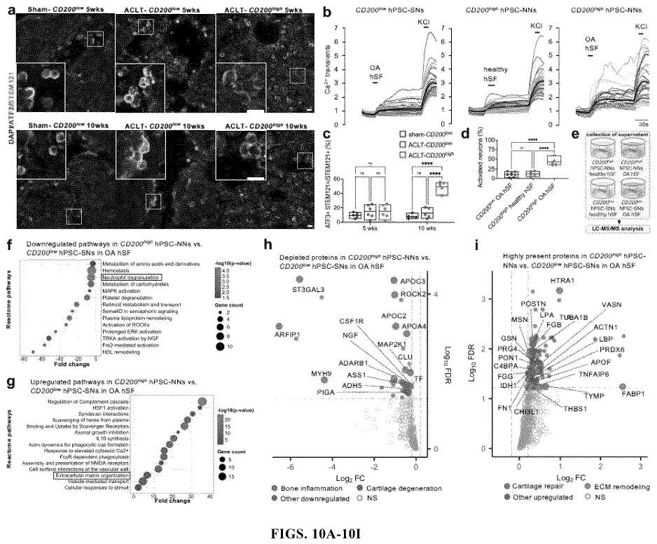

FIGS. 9A-9K. illustrate in vivo survival and function of SCN9A+, TRPV1+, and MRGPRX1+ neurons. To explore in vivo survival and functional integration abilities, we transplanted FACS-purified hPSC-derived mature TRPV1::GFP+, SCN9A::GFP+, and MRGPRX1::GFP+ hPSC-SNs, and differentiating neural crest cells into the right L4 DRG of adult Rag2^{emlhera} Il2^{emlhera}/HblCrl rats (FIG. 9A). An expression cassette harboring TdTomato and excitatory (Gq) designer receptors activated by designer drugs (Gq-DREADDs) was packaged into AAV PHP.s and later transduced into hPSC-SNs prior to the injection to allow for chemogenic modulations (FIG. 9B-9C). When maintained in vitro, transduced TRPV1::GFP+, SCN9A::GFP+, and MRGPRX1::GFP+ donor hPSC-SNs showed extended axons (up to 800 μm-1 mm) 5 days after plating with visible groVv1h cone (FIG. 9D). Left (contralateral control group) and right (injected group) L4 DRGs were isolated from animals 5 weeks after transplantation surgery to examine the survival of hPSC-derived donor neurons. While GFP background was low in the control DRG (FIG. 9E), the injected DRGs and attached nerve roots harbored clusters of GFP-expressing hPSC-derived donor neurons with axonal sprouting (FIG. 4F). To investigate whether the survived hPSC-derived donor neurons could establish functional connections with endogenous neurons, we examined the behavioral consequences of rats 4 weeks after the initial hPSC-SN transplantation (FIGS. 9G-9H). Spontaneous pain behaviors were assessed using video-observation of rats after intra-peritoneal administration of the DREADD activator CNO and PBS. Duration of right hind paw licking was significantly heighted after CNO administration compared to PBS (FIG. 9I). Mechanical allodynia was examined using Von Frey tests 7 days after the initial CNO administration or acutely after CNO administration. The injected side displayed a significant decrease in threshold 7 days after the initial CNO administration and 1 hour after acute CNO administration FIGS. 9J-9K). Taken together, these data show that injected hPSC-SNs could survive in vivo and modulate endogenous neuronal activities by sensitization or direct excitation (FIGS. 9J-9K).

In conclusion, the study presents that TRPV1::GFP+, SCN9A::GFP+ and MRGPRX1::GFP+ hPSC-SNs can be generated and confirmed their functionality with multiple pain and itching stimuli. The hPSC-derived TRPV1::GFP+ and SCN9A::GFP+ neurons also display great potential for in vivo survival and functionality after transplantation. These data provide new insight on stem cell fate determination processes toward a specific sensory neuronal subtype, open new avenues for chronic pain therapeutics using cell therapy, and build a foundation to develop novel drug candidates for managing persistent pain/itching conditions. The various embodiments described above are provided by way of illustration only and should not be construed to limit the scope of the disclosure. Various modifications and changes may be made to the principles described herein without following the example embodiments and applications illustrated and described herein, and without departing from the spirit and scope of the disclosure. For example, unless otherwise explicitly indicated, the steps of a process or method may be performed in an order other than the example embodiments discussed above. Likewise, unless otherwise indicated, various components may be omitted, substituted, or arranged in a configuration other than the example embodiments discussed above.

### Example 3. CD200^{high} hPSC-NNs Promote Joint Homeostasis by Depleting Inflammatory Factors and Secreting Reparative Factors in Human and Mouse Joint Tissues

Another study sought to determine whether the analgesic and anti-inflammatory effects of CD200^{high} hPSC-NNs were mediated through the direct binding and sequestration of inflammatory mediators. To assess whether CD200^{high} hPSC-NNs were activated in vivo in response to inflammatory stimuli, immunostaining was performed for the neuron-specific injury marker ATF3 in STEM121+ transplanted cells. Notably, CD200^{high} hPSC-NNs displayed significantly elevated ATF3 expression compared with CD200^{low} hPSC-SNs in ACLT animals, consistent with the hypothesis that CD200^{high} hPSC-NNs are activated in vivo by endogenous inflammatory mediators (FIGS. 10A and 10C). To further test their responsiveness to inflammatory cues, calcium imaging was performed following acute exposure to synovial fluid from OA patients. CD200^{high} hPSC-NNs exhibited rapid and robust calcium transients upon stimulation with synovial fluid from two independent OA donors, whereas CD200^{low} hPSC-SNs showed delayed and significantly attenuated responses with a significantly lower proportion of responsive cells, comparable to responses elicited by pooled synovial fluid from healthy donors (FIGS. 10B and 10D).

To further elucidate the mechanisms underlying the analgesic and immune modulatory effects of CD200^{high} hPSC-NNs, a proteomic analysis was performed on conditioned supernatants collected from CD200^{high} hPSC-NNs and CD200^{low} hPSC-SNs following overnight incubation with synovial fluid from two osteoarthritic patients (OA hSF) or pooled healthy donors (healthy hSF) (FIG. 10E). Differential expression and gene ontology analysis of the proteomic profile revealed a significant downregulation of proteins implicated in bone inflammation and cartilage degeneration in OA hSF incubated with CD200^{high} hPSC-NNs compared with CD200^{low} hPSC-SNs. Among inflammation-associated proteins, ST3 beta-galactoside alpha-2,3-sialyltransferase 3 (ST3GAL3) activates the Toll-like receptor 9 (TLR9)/MyD88 signaling pathway, whereas the apolipoproteins apolipoprotein C3 (APOC3) and apolipoprotein C2 (APOC2) promote inflammasome activation and pro-inflammatory macrophage polarization, respectively. Among the proteins implicated in cartilage degeneration, nerve growth factor (NGF) stimulates the production of MMPs and facilitates neurovascular invasion, while Rho-associated coiled-coil kinase 2 (ROCK2) and mitogen-activated protein kinase 1 (MAP2K1), two essential proteins in the Rho/ROCK and MEK/ERK pathways, mediate degradation of chondrocytes and the surrounding extracellular matrix (ECM) (FIGS. 10F and 10H). Conversely, proteins and pathways implicated in cartilage repair and ECM remodeling were markedly enriched in the OA hSF group incubated with CD200^{high} hPSC-NNs compared with CD200^{high} hPSC-SNs. Notably, thrombospondin-1 (THBS1) exerted pro-chondrogenic effects31 while chitinase 3 like 1 (CHI3L1) inhibited cartilage matrix degeneration by modulating the TLR4-MAPK-STAT1 pathway (FIGS. 10G and 10I).

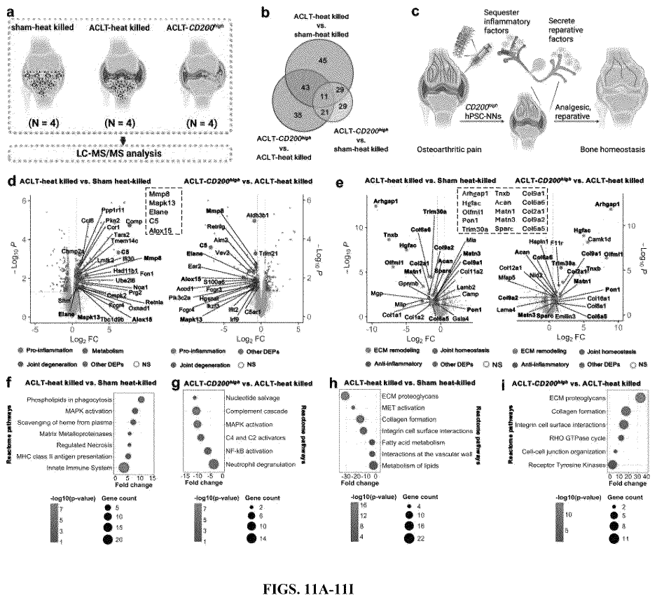

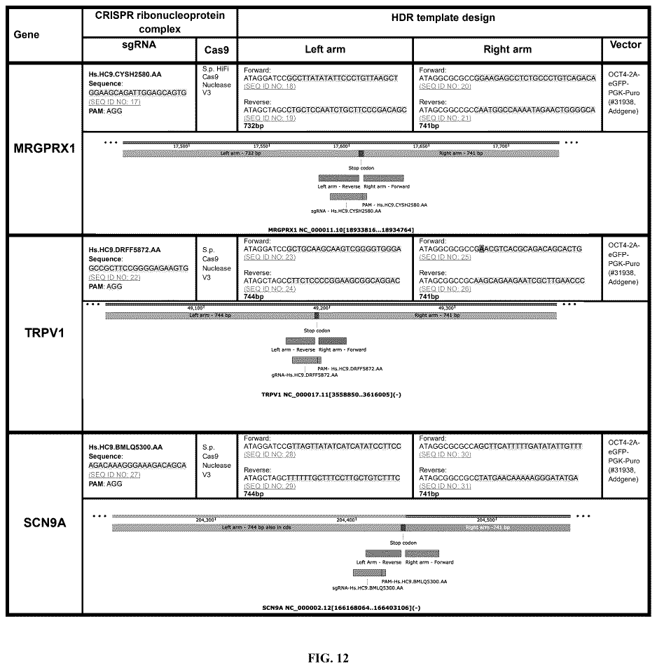

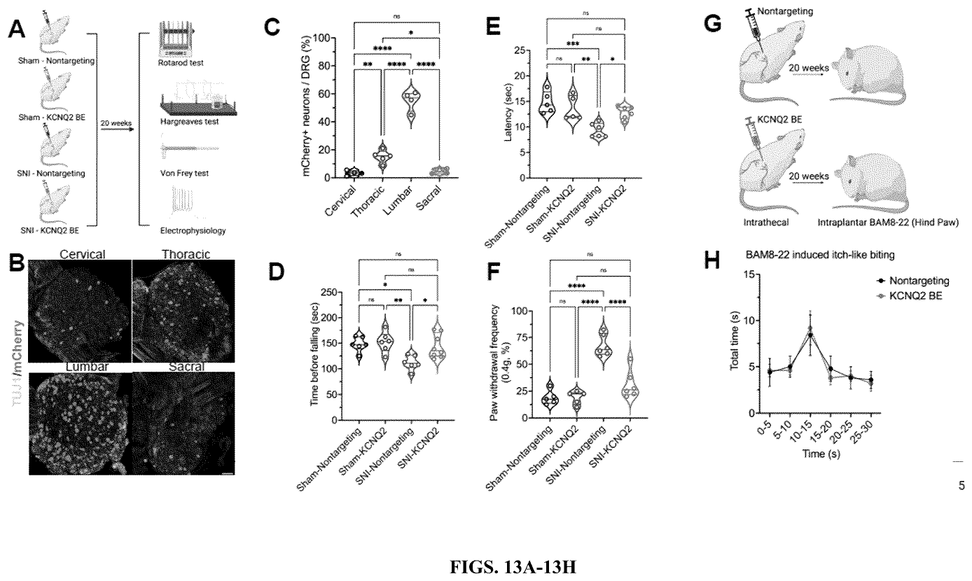

To control for passive release of intracellular contents from dead cells, proteomic analysis was conducted on knee joints harvested 10 weeks post-injection from sham-heat killed, ACLT-heat killed, and ACLT-CD200^{high} groups to identify differentially regulated human and mouse proteins (FIG. 11A). Due to the predominance of mouse-derived proteins in the joint tissue, the detection of human proteins fell below the threshold of proteomic reliability. Quantification of differentially expressed proteins across the three groups revealed a divergence of the proteomes between the ACLT-heat killed and sham-heat killed groups, and a remarkable similarity between the proteomes of ACLT-CD200^{high} and sham-heat killed animals (FIG. 11B). Differential expression and gene ontology analysis showed factors implicated in inflammation and joint degeneration were significantly enriched in ACLT-heat killed animals but, strikingly, were reversed in ACLT-CD200^{high} animals. Among these proteins, matrix metalloproteinase-8 (Mmp8) promotes chondrocyte apoptosis and cartilage degradation33, and mitogen-activated protein kinase 13 (Mapk13) increases expression of catabolic enzymes34 (FIGS. 11D, 11F, and 11G). Conversely, proteins and pathways involved in anti-inflammation, ECM remodeling, and joint homeostasis were suppressed in ACLT-heat killed animals but restored in ACLT-CD200^{high} animals. Among these proteins, olfactomedin 1 (Olfm1) inhibits osteoclast differentiation, while ECM components including matrilin-1 (Matn1), matrilin-1 (Matn3), tenascin XB (Tnxb), and multiple collagen peptides are essential for cartilage integrity (FIGS. 11E, 11H, and 11I). Notably, among the genes enriched in both OA hSF and mouse knee joints following CD200^{high} hPSC-NN treatment, paraoxonase 1 (Pon1) is an enzyme that protects lipoproteins from oxidative modifications. Together, these data suggest that CD200^{high} hPSC-NNs act through a dual mechanism in OA, simultaneously sequestering pro-inflammatory mediators and secreting factors that promote bone and ECM remodeling, thereby creating a reparative, anti-inflammatory microenvironment in human and mouse joint tissues (FIG. 11C).

### CONCLUSION

In sum, these findings present an alternative application of hPSC-derived cells that diverge from this traditional regenerative paradigm. Furthermore, the results demonstrate that hPSC-NNs can function as therapeutic agents by modulating the inflammatory microenvironment through both ligand sequestration and the release of reparative factors in mouse and human joint tissues instead of simply replacing damaged tissue.

### Example 4: Targeted Base Editor Reduces Pain Indicators in Animal Study

FIG. 13A depicts an animal model in which mice with or without spared nerve injury (SNI) received intrathecal injection of either a targeted base editor or a nontargeting control, followed by pain-related functional assessment 20 weeks later. The targeted base editor comprises a CRISPR sgRNA sequence that targets KCNQ2. The nontargeting control comprises a control sgRNA sequence that has no known genomic target as a control sequence.

FIGS. 13B-C show uptake of the KCNQ2 gene editor across cervical, thoracic, lumbar, and sacral spinal regions using mCherry-labeled TUJ1 antibody. FIGS. 13D-F present pain-related measurements for each cohort; notably, FIG. F shows that SNI mice treated with the KCNQ2 base editor exhibit reduced paw withdrawal efficiency—comparable to uninjured mice—relative to control-treated SNI mice. In FIGS. C-F and throughout the present disclosure, “*” refers to a p-value of less than 0.05, “**” refers to a p-value of less than 0.01, “***” refers to a p-value of less than 0.001, and “****” refers to a p-value of less than 0.0001.

### Example 5. KCNQ2 Base Editor Shows No Significant Effect on Itch Behavior

FIG. 13G illustrates an animal model in which mice received intrathecal injection of either a KCNQ2-targeted base editor or a nontargeting control, with intraplantar BAM8-22 injection and measurement of itch-like biting performed 20 weeks later. Results are shown in FIG. 13H.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

1. An isolated population of cells comprising pluripotent stem cell-derived sensory neurons (PSC-SNs) or descendants thereof, the PSC-SNs expressing one or more of a sensory neuron marker and a pan neuronal marker.

2. The isolated population of cells of claim 1, wherein the PSC-SNs respond to a stimulus that is nociceptive and pruriceptive.

3. The isolated population of cells of claim 1, comprising cells that express one or more of TRPV1, SCN9A, MRGPRX1, and KCNQ2.

4. The isolated population of cells of claim 1, comprising cells that express CD200^{high}.

5. The isolated population of cells of claim 1, wherein at least 40% of cells in the isolated population of cells express CD200^{high}.

6. The isolated population of cells of claim 1, wherein the PSC-SNs comprise PSC-SNs that are derived from a human (hPSC-SNs).

7. The isolated population of cells of claim 1, wherein the PSC-SNs comprise pluripotent stem cell-derived nociceptive sensory neurons (PSC-NSNs).

8. A method, comprising: administering pluripotent stem cell-derived sensory neurons (PSC-SNs) to a subject, the PSC-SNs expressing one or more of a sensory neuron marker and a pan neuronal marker.

9. The method of claim 8, wherein the method is a method to treat pain.

10. The method of claim 8, wherein the method is a method to treat osteoarthritis.

11. The method of claim 8, wherein the administering comprises administering the cells to a joint of the subject.

12. The method of claim 11, wherein the joint is a knee joint.

13. The method of claim 8, wherein the pain comprises one or more of chronic pain, nociceptive pain, and pruriceptive pain.

14. The method of claim 8, wherein the method reduces expression of matrix metalloproteinase 13 (MMP13) in the subject by at least about 10%.

15. A composition for base editing target gene mutations, comprising:
a base editor; and
a guide RNA (gRNA),
the base editor and the gRNA targeting at least one gene, the at least one gene comprising SCN9A, TRPV1, MRGPRX1, or KCNQ2.

16. The composition of claim 15, wherein the gRNA comprises a sequence selected from any one of SEQ ID NOs: 82-97.

17. The composition of claim 15, wherein the gRNA comprises a spacer sequence selected from any one of SEQ ID NOs: 17, 22, 27, 32, 35, 38, 41, 44, 47, 50, 53, 56, or 59-81.

18. The composition of claim 15, wherein the base editor comprises ABE8e, ABE8e(V106W), ABE9, ABE20, ABE7.10, SaKKHABE8e, SauriCas9-ABE8e, CjCas9-ABE8e, Nme2Cas9-ABE8e, or SaCas9-ABE8e.

19. The composition of claim 15, wherein the base editor comprises Cas9 endonuclease.

20. The composition of claim 19, wherein the Cas9 endonuclease comprises Type II Cas9.
